(19) United States
(12) Patent Application Publication
(10) Pub. No.: US 2025/0084423 A1
(43) Pub. Date: Mar. 13, 2025

(54) BIOSYNTHESIS OF SULFUR-CONTAINING COMPOUNDS USING GENETICALLY MODIFIED BACTERIA

(71) Applicant: Chemical Evolution Ltd., Calgary (CA)

(72) Inventors: Alejandra ENRIQUEZ, Calgary (CA); Trevor RANDALL, Calgary (CA); Dustin LILLICO, Calgary (CA); Markus WEISSENBERGER, Calgary (CA)

(21) Appl. No.: 18/771,998

(22) Filed: Jul. 12, 2024

(30) Foreign Application Priority Data
Jul. 14, 2023 (CA) 3206613

(51) Int. Cl. C12N 15/77; C12N 9/02; C12N 9/80

(52) U.S. Cl. CPC C12N 15/77; C12N 9/0069; C12N 9/80; C12Y 113/11019

(57) ABSTRACT

A genetically modified prokaryotic cell which comprises: a cysteamine dioxygenase (ADO) polypeptide sequence which has at least 70% sequence coverage to SEQ 2, and at least 25% sequence identity to SEQ 2; and a vanin (VNN) polypeptide sequence selected from the group consisting of: a vanin-1 (VNN1) polypeptide sequence which has at least 70% sequence coverage to SEQ 4, and at least 25% sequence identity to SEQ 4; a vanin-2 (VNN2) polypeptide sequence which has at least 70% sequence coverage to SEQ 84, and at least 25% sequence identity to SEQ 84; and a vanin-3 (VNN3) polypeptide sequence which has at least 70% sequence coverage to SEQ 128 and at least 25% sequence identity to SEQ 128.

### REFERENCE TO AN ELECTRONIC SEQUENCE LISTING

The contents of the electronic sequence listing (20241129_SequenceListing_ST26_23156196US1.xml; Size: 247,524 bytes; and Date of Creation: Nov. 29, 2024) is herein incorporated by reference in its entirety.

### FIELD OF THE INVENTION

The present invention relates to a method to produce taurine through genetically manipulated bacteria to ferment a sugar source and a sulfur source into taurine using naturally present or added metabolic pathways.

### BACKGROUND OF THE INVENTION

Taurine is an amino acid that has been shown to be beneficial to human and animal health and development, and thus it is commonly found supplemented into animal feed for livestock, as a stimulant in energy drinks, sold directly as a supplement, and in baby formula. Currently, most of the taurine on the market is chemically synthesized from either ethylene oxide or monoethanolamine (MEA). Manufacturing taurine using ethylene oxide, although the most common, comes with several drawbacks including its toxicity, volatility, and explosive potential, in addition to the severe conditions of temperature and pressure under which the reaction takes place. On the other hand, the industrial production of taurine from MEA is a two-step batch process in which the first step is the reaction of MEA with sulfuric acid to produce the ester 2-aminoethyl hydrogen sulfate (AES) and then second involving a subsequent reaction of AES with a sulfite reagent. This general manufacturing process has limitations which includes the yield of the intermediate esters involved within the above-mentioned reactions and the need for high temperatures and corrosive acids to achieve production. To circumvent these limitations, the proposed invention is intended to utilize a prokaryotic biological system for the production of taurine through specific genetic modifications that incorporate pathways exclusively associated with eukaryotes. Taurine production by eukaryotes is facilitated by very distinct sets of metabolic pathways which includes the conversion of methionine and cysteine via cysteine sulfinic acid decarboxylase (CSAD) coupled with the oxidation of hypotaurine to generate taurine as the final step within the pathway.

U.S. patent application No. US 2019/0153463 A1 describes an approach to produce or increase taurine and/or hypotaurine production in prokaryotes or eukaryotes. More particularly, the invention relates to genetic transformation of organisms with algal, microalgal or fungal genes that encode proteins that catalyze the conversion of sulfur-containing compounds such as sulfate or cysteine to taurine. The invention describes methods for the use of polynucleotides for cysteine dioxygenase-like (CDOL), sulfinoalanine decarboxylase-like (SADL), cysteine sulfate/decarboxylase or a portion of the cysteine synthetase/PLP decarboxylase (partCS/PLP-DC) polypeptide in bacteria, alga, yeast, or plants to produce or enhance taurine and/or hypotaurine formation. The preferred embodiment of the invention is in plants, but other organisms may be used. The direct generation or alteration of taurine and/or hypotaurine in plants could be used as nutraceutical, pharmaceutical, or therapeutic compounds. Furthermore, both taurine and hypotaurine could also be utilized to enhance both the growth and health of animals by directly being used as a food source or an added supplement to feed.

U.S. Pat. No. 10,874,625 B2 describes an approach to increase taurine or hypotaurine production in prokaryotes. More particularly, the invention relates to genetic transformation of organisms with genes that encode proteins that catalyze the conversion of cysteine to taurine, methionine to taurine, cysteamine to taurine, or alanine to taurine. The invention describes methods for the use of polynucleotides that encode cysteine dioxygenase (CDO) and sulfinoalanine decarboxylase (SAD) polypeptides in prokaryotes to increase taurine, hypotaurine or taurine precursor production. The preferred embodiment of the invention is in plants, but other organisms may be used. Increased taurine production in prokaryotes could be used as nutraceutical, pharmaceutical, or therapeutic compounds or as a supplement in animal feed.

U.S. Pat. No. 11,220,691 B2 describes an approach to produce or increase hypotaurine or taurine production in unicellular organisms. More particularly, the invention relates to genetic modification of unicellular organisms that include bacteria, algal, microalgal, diatoms, yeast, or fungi. The invention relates to methods to increase taurine levels in the cells by binding taurine or decreasing taurine degradation. The invention can be used in organisms that contain native or heterologous (transgenic) taurine biosynthetic pathways or cells that have taurine by enrichment. The invention also relates to methods to increase taurine levels in the cells and to use the said cells or extracts or purifications from the cells that contain the invention to produce plant growth enhancers, food, animal feed, aquafeed, food or drink supplements, animal-feed supplements, dietary supplements, health supplements or taurine.

In J. Agric. Food Chem. 2018, 66, 51, 13454-13463, Joo et al. reported, for the first time in bacteria, the production of taurine in metabolically engineered Corynebacterium glutamicum. The taurine-producing strain was developed by introducing CS, CDO1, and CSAD genes. Interestingly, while the control strain could not produce taurine, the engineered strains successfully produced taurine via the newly introduced metabolic pathway.

U.S. Pat. No. 11,326,171 B2 provides non-naturally occurring microorganisms that produce taurine and/or taurine precursors, e.g., hypotaurine, sulfoacetaldehyde, or cysteate, utilizing exogenously added enzyme activities. The invention disclosed therein relates to methods of producing taurine and/or taurine precursors in microbial cultures, and feed and nutritional supplement compositions that include taurine and/or taurine precursors produced in the microbial cultures, such as taurine-and/or taurine precursor-containing biomass, are also provided.

FIG. 1 is a schematic representation of various known pathways to make taurine. In contrast to the plethora of research conducted surrounding the process utilizing cysteine dioxygenase (CDO) and sulfinoalanine/cysteine sulfinic acid decarboxylase (SAD/CSAD), there are no commercial embodiments which employ the pathway from cysteamine to hypotaurine.

In light of the state of the art, there still exists a need to develop a large scale, an environmentally conscious process for the production of sulfur-containing compounds (such as taurine) which is a cost-effective alternative to current chemical and biological processes.

### SUMMARY OF THE INVENTION

According to a first aspect of the present invention, there is provided a novel biosynthetic pathway from cysteamine to hypotaurine which is engineered within a biological system and overcome the high energy intensity of chemical processes.

The embodiments of the present invention are generally related to novel organisms for the production of sulfur-containing compounds in prokaryotic organisms. More particularly, the invention encompasses methods for the genetic modifications of bacterial organisms which allows the organism to produce said sulfur-containing compounds through the introduction of eukaryotic genes into the prokaryotic genome or cell. Through the inclusion of said eukaryotic genes into a prokaryotic genome or cell, there are proposed novel metabolic pathways beyond those present natively in the prokaryotic organism itself.

According to a preferred embodiment of the present invention, said sulfur-containing compound of interest is selected from the group comprising cysteamine, cysteine, hypotaurine, and taurine.

According to an aspect of the present invention, there is provided a polynucleotide and thus polypeptide sequences from eukaryotes and bacteria to allow for the expression of proteins in bacteria that allow for the production of a sulfur-containing compound.

According to a preferred embodiment of the present invention, there is provided a genetically modified prokaryotic cell which comprises a cysteamine dioxygenase (ado) polynucleotide sequence SEQ 1, which has at least 70% sequence coverage to SEQ 1, and has-at least 70% sequence identity to SEQ 1.

According to a preferred embodiment of the present invention, there is provided a genetically modified prokaryotic cell which comprises a cysteamine dioxygenase (ado) polynucleotide sequence, wherein said ado polynucleotide sequence is selected from the group consisting of SEQ 1; SEQ 22; SEQ 23; SEQ 24; SEQ 25; SEQ 26; and SEQ 27.

According to a preferred embodiment of the present invention, SEQ 1, upon transcription and translation, provides a cysteamine dioxygenase (ADO) polypeptide sequence of SEQ 2. According to a preferred embodiment of the present invention, there is provided a genetically modified prokaryotic cell which comprises an ADO polypeptide sequence which has at least 70% sequence coverage to SEQ 2, and at least 25% sequence identity to SEQ 2.

According to a preferred embodiment of the present invention, there is provided a genetically modified prokaryotic cell wherein said cell comprises a cysteamine dioxygenase (ADO) polypeptide sequence obtained through the transcription and translation of an ado polynucleotide mentioned herein, and wherein said ADO polypeptide sequence is selected from the group consisting of: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40; SEQ 41; and SEQ 42.

According to another aspect of the present invention, there is provided a genetically modified prokaryotic cell which comprises a vanin-1 (vnn1) polynucleotide sequence which has at least 70% sequence coverage to SEQ 3 or SEQ 69, and at least 70% sequence identity to SEQ 3 or SEQ 69.

According to a preferred embodiment of the present invention, said vnn1 polynucleotide sequence is selected from the group consisting of SEQ 3; SEQ 43; SEQ 44; SEQ 45; SEQ 46; SEQ 47; SEQ 48; SEQ 49; SEQ 50; SEQ 51; SEQ 52; and SEQ 69.

According to a preferred embodiment of the present invention, SEQ 3 or SEQ 69, upon transcription and translation, provides a vanin-1 (VNN1) polypeptide sequence SEQ 4. According to a preferred embodiment of the present invention, there is provided a genetically modified prokaryotic cell which comprises an VNN1 polypeptide sequence which has at least 70% sequence coverage to SEQ 4, and at least 25% sequence identity to SEQ 4.

According to a preferred embodiment of the present invention, the VNN1 polypeptide sequenceis selected from the group consisting of: SEQ 4; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; and SEQ 68.

According to another aspect of the present invention, there is provided a genetically modified prokaryotic cell which comprises a vanin-2 (vnn2) polynucleotide sequence which has at least 70% sequence coverage to SEQ 70, and at least 70% sequence identity to SEQ 70.

According to a preferred embodiment of the invention, the vanin-2 (vnn2) polynucleotide sequence is used in place of the vnnl polynucleotide sequence. Said vnn2 polynucleotide sequence is selected from the group consisting of: SEQ 70; SEQ 71; SEQ 72; SEQ 73; SEQ 74; SEQ 75; SEQ 76; SEQ 77; SEQ 78; SEQ 79; SEQ 80; SEQ 81; SEQ 82; and SEQ 83.

According to a preferred embodiment of the present invention, SEQ 70, upon transcription and translation, provides a vanin-2 (VNN2) polypeptide sequence of SEQ 84. According to a preferred embodiment of the present invention, there is provided a genetically modified prokaryotic cell which comprises an VNN2 polypeptide sequence which has at least 70% sequence coverage to SEQ 84, and at least 25% sequence identity to SEQ 84.

According to a preferred embodiment of the invention, the VNN2 polypeptide sequence can be used in place of the VNN1 polypeptide sequence. Said VNN2 polypeptide sequence is selected from the group consisting of: SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; SEQ 97; SEQ 98; SEQ 99; SEQ 100; SEQ 101; SEQ 102; SEQ 103; SEQ 104; SEQ 105; SEQ 106; SEQ 107; SEQ 108; SEQ 109; and SEQ 110.

According to another aspect of the present invention, there is provided a genetically modified prokaryotic cell which comprises a vanin-3 (vnn3) polynucleotide sequence which has at least 70% sequence coverage to SEQ 111, and at least 70% sequence identity to SEQ 111.

According to a preferred embodiment of the invention, the vanin-3 (vnn3) polynucleotide sequence can be used in place of the vnn1 polynucleotide sequence. Said vnn3 polynucleotide sequence can be selected from the group consisting of: SEQ 111; SEQ 112; SEQ 113; SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; and SEQ 127.

According to a preferred embodiment of the present invention, SEQ 111, upon transcription and translation, provides a vanin-3 (VNN3) polypeptide sequence of SEQ 128. According to a preferred embodiment of the present invention, there is provided a genetically modified prokaryotic cell which comprises an VNN3 polypeptide sequence which has at least 70% sequence coverage to SEQ 128, and at least 25% sequence identity to SEQ 128.

According to a preferred embodiment of the invention, the VNN3 polypeptide sequence can be used in place of the VNN1 polypeptide sequence. Said VNN3 polypeptide sequence can be selected from the group consisting of: SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; SEQ 146; SEQ 147; SEQ 148; SEQ 149; SEQ 150; SEQ 151; SEQ 152; and SEQ 153.

According to a preferred embodiment of the present invention, the prokaryotic cell further comprises a promoter and RBS sequence which drives gene expression, wherein the genetic material for the promoter/RBS sequences comprises at least one or another of the following: SEQ 5; SEQ 6; SEQ 7; SEQ 8; SEQ 9; SEQ 10 SEQ 11; SEQ 12; SEQ 13; SEQ 14; SEQ 15; SEQ 16; SEQ 17; SEQ 18; SEQ 19; SEQ 20; and SEQ 21.

According to a preferred embodiment of the present invention, there is provided a genetically modified prokaryotic cell which comprises:
a cysteamine dioxygenase (ado) polynucleotide sequence which has at least 70% sequence coverage to SEQ 1, and at least 70% sequence identity to SEQ 1; and
a vanin (vnn) polynucleotide sequence selected from the group consisting of:
   a vanin-1 (vnn1) polynucleotide sequence which has at least 70% sequence coverage to SEQ 3 or SEQ 69, and at least 70% sequence identity to SEQ 3 or SEQ 69;
   a vanin-2 (vnn2) polynucleotide sequence which has at least 70% sequence coverage to SEQ 70, and at least 70% sequence identity to SEQ 70; and
   a vanin-3 (vnn3) polynucleotide sequence which has at least 70% sequence coverage to SEQ 111, and at least 25% sequence identity to SEQ 111.

According to a preferred embodiment of the present invention, there is provided a genetically modified prokaryotic cell which comprises:
a cysteamine dioxygenase (ADO) polypeptide sequence which has at least 70% sequence coverage to SEQ 2, and at least 25% sequence identity to SEQ 2; and
a vanin (VNN) polypeptide sequence selected from the group consisting of:
   a vanin-1 (VNN1) polypeptide sequence which has at least 70% sequence coverage to SEQ 4, and at least 25% sequence identity to SEQ 4;
   a vanin-2 (VNN2) polypeptide sequence which has at least 70% sequence coverage to SEQ 84, and at least 25% sequence identity to SEQ 84; and
   a vanin-3 (VNN3) polypeptide sequence which has at least 70% sequence coverage to SEQ 128 and at least 25% sequence identity to SEQ 128.

Preferably, the prokaryotic cell is a bacterial cell. Preferably, the cell is selected from the group consisting of the genera: Brevibacterium, Bacillus, Corynebacterium, Escherichia, Lactococcus, Pseudomonas, Rhodococcus, and Serratia. More preferably, the cell belongs to the genus Corynebacterium. Even more preferably, the bacterial organism is Corynebacterium glutamicum.

According to a preferred method of the present invention, genes and promoter sequences were introduced into the genome or genetics of the organism using a genetic engineering method such as two-step allelic exchange or via introduction into the bacteria via an expression plasmid. Natural unmodified promoters and ribosomal binding sites from the bacterial expression strain or synthetic or modified promoters and ribosomal binding sites were attached to the genes upstream of the gene's start codon to drive gene expression within the bacterium.

According to another aspect of the present invention, there is provided a method to transfer these genes from cloning vectors into the organisms of interest. Preferably, these methods are modelled after the protocol seen for the knock in or knock out of genes in a different bacterium, Pseudomonas aeruginosa, using the aforementioned two step allelic exchange. However, the listed methods are in no way meant to exclude the use of other methods, such as CRISPR cloning, from being used.

According to a preferred embodiment of the present invention, the bacterial cells were genetically modified by inserting a native bacterial or synthetic promoter sequence into the bacterial genome, immediately followed by a gene(s) related to the production of sulfur-containing compounds and/or their precursors. According to a preferred embodiment of the present invention, the native promoter and polynucleotide sequence also includes the promoter and ribosomal binding site (RBS) including various combinations from the following genes: serine hydroxymethyltransferase (glyA) (P_{glyA}), superoxide dismutase (SOD) gene (P_{SOD}), Phosphoglycerate kinase (P_{pgk}), the EF-Tu transcription factor (P_{tuf}). Fructose-bisphosphate aldolase (P_{fbaA}), Aspartokinase (P_{lysC}), Transketolase (Pₜₖₜ), Glutamine synthetase (P_{glnA}), Pyruvate carboxylase (P_{pyc}), Homoserine dehydrogenase (Pₕₒₘ), 6-phosphogluconate dehydrogenase (P_{gnd}), Diaminopimelate decarboxylase (P_{lysA}), Aspartate aminotransferase (P_{aspB}), Meso-diaminopimelate D-dehydrogenase (P_{ddh}), or 4-hydroxy-tetrahydrodipicolinate reductase (P_{dapB}), as well as the artificial promoter and RBS sequences for Tac (P_{tac}), however this list of promoter and RBS sequences is in no way meant to limit the native or synthetic promoter and/or RBS sequences that can be used. In other embodiments, the promoter and RBS polynucleotide sequence to regulate hypotaurine/taurine production can specifically be the native promoter for the serine hydroxymethyltransferase (glyA) gene (P_{glyA}) present in C. glutamicum. In other preferred embodiments, the promoter and RBS polynucleotide sequence to regulate hypotaurine/taurine production can specifically be the native promoter for the superoxide dismutase (SOD) gene (P_{SOD}), obtained from C. glutamicum. In another embodiment of the invention, the artificial promoter and RBS sequences for Tac (P_{tac}) may also be used to drive production of taurine biosynthetic-related genes. Preferably the native promoter and RBS used are P_{glyA}, P_{SOD}, and P_{tac}.

According to a preferred embodiment of the present invention, the utilized native or synthetic promoter sequence is followed by the polynucleotide sequence for genes related to the production of sulfur-containing compounds, such as cysteamine dioxygenase (ado) or vanin-1 (vnn1) natively found in eukaryotic organisms. In some embodiments, the ado and vnn1 genes can be acquired from eukaryotic organisms such as Sus scrofa, Homo sapiens, Ursus maritimus, Lutra lutra, Nycticebus coucang, Mus musculus, Salvelinus alpinus, Phrynosoma platyrhinos, Vombatus ursinus, Bucco capensis, Notechis scutatus, Sinocyclocheilus anshuiensis, Salmo salar, Marmota monax, Clupea harengus, and Harpia harpyja although the listed organisms are only given as examples and are in no way meant to limit what organisms these genes can be acquired from. In a preferred embodiment of this invention, the ado and vnn1 genes are obtained from the organism Sus scrofa. In another preferred embodiment of the present invention. the native or synthetic promoter sequence is used to bolster the production of proteins and/or molecules related to the production of precursors for taurine production.

### BRIEF DESCRIPTION OF THE ACCOMPANYING FIGURE

The invention may be more completely understood in consideration of the following description of various embodiments of the invention in connection with the accompanying figure, in which:

FIG. 1 is a schematic representation of various known pathways to make sulfur-containing compounds.

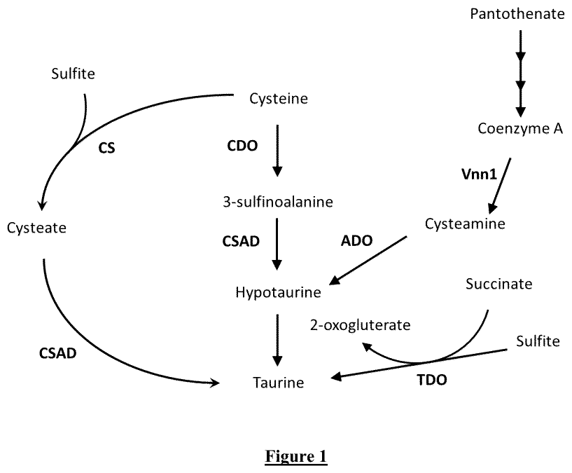

### DETAILED DESCRIPTION OF THE INVENTION

The invention described herein addresses genetic modifications to bacterial strains allowing for the production of a sulfur-containing compound from an inexpensive feedstock using bacterial species modified with the eukaryotic ado (SEQ 1) and vanin (SEQ 3, SEQ 69, SEQ 70 or SEQ111) polynucleotides encoding for the polypeptide sequences ADO (SEQ 2) and VNN (SEQ 4, SEQ 84, or SE 128).

Provided herein are genetically engineered bacteria that can produce hypotaurine, taurine, or taurine precursors from a sugar source and a sulfur source. Also provided are methods to genetically engineer and culture hypotaurine and taurine producing bacteria.

### Definitions

Within the context of the present invention all terms and technical parameters described fall within their commonly known meanings as known by individuals within the region of science that the proposed invention is associated with, unless otherwise stated. Furthermore, unless otherwise indicated, all techniques utilized within this invention are commonly conducted within the fields of molecular biology, cell biology, biochemistry, and microbiology.

A polynucleotide within the context of the present invention is defined as the collection of individual nucleotides in any organization or size that relates to the DNA sequence.

A polypeptide within the context of the present invention is defined as the combination of multiple peptides of any organization or size that relates to the amino acid sequence. The term polypeptide and protein within the context of this invention can be used interchangeably.

A vector within the context of the present invention refers to the composition of a polynucleotide with the intended purpose of introducing nucleic acids into one or more organism types. Vectors are further defined based on their functional purpose and can be designated as expression vectors, cloning vectors, plasmids, or shuttle vectors.

The term “expression” within the context of the present invention refers to the generation of a polypeptide sequence which is produced based on its polynucleotide sequence or gene.

An “expression vector” within the context of the present invention references a polynucleotide sequence containing a coding sequence or gene that enhances or promotes the generation of a polypeptide when introduced into an organism. An expression vector contains all the necessary polypeptide producing features such as a promoter and ribosomal binding site which allow for the production (or expression) of a desired gene due to transcription and translation processes.

A promoter within the context of the present invention is used to describe the nucleic acid sequence for the regulation and binding of polymerases for the purpose of transcribing a gene. This promoter can be native to an organism, or a non-endogenous promoter can be introduced into an organism to alter the regulation of gene expression.

The term gene refers to a DNA sequence that encodes for a specific polypeptide sequence. A gene can include both sequences between coding regions (introns) and the encoding sequence itself (exon).

The term recombinant within the context of the present invention refers to the modification or alteration of a sequence associated with either a polypeptide or polynucleotide sequence. Recombination can be utilized for altering expression and coding segments of a gene of interest that would produce a non-native or non-naturally occurring product.

The term exogenous refers to the addition of either polypeptide and/or polynucleotide molecules that are not normally found within the organism. This includes any un-altered or altered genes and/or proteins that are not found conventionally within an organism.

The term homology refers to the level of similarity between two or more polypeptide or polynucleotide sequences.

The terms transfection, transformation, or introduced refer to the addition of polynucleotide sequence(s) that would normally be considered exogenous to the organism. This can include the addition of a polynucleotide directly to the genome of an organism or the transfer of a plasmid and/or vector to be maintained within the organism.

Within the context of the present invention, the terms native or natural refers to polypeptide and/or polynucleotides present within the organism prior to any modification. These native or naturally occurring polypeptides and/or polynucleotides would be present or produced by the organism without any external alterations.

The term metabolic pathway refers to the subsequential biochemical reactions involved in the formation of a biologically relevant product within an organism.

Within the context of the present invention the terms “knock-in” and “knock-out” refer to the addition or removal of DNA sequences within an organism and can also be interchangeable with the terms insertion and deletion, respectively.

A coding sequence within the context of the present invention refers to a sequence of polynucleotides or DNA that facilitates the generation of a protein through transcription and translational processes (also known as transcribed and translated).

Genetic modification or related statements herein refer to the alteration of the genetic code of an organism which includes the insertion or deletion of DNA sequences within an organism. Within the context of the present invention, genetic modification could include insertion and maintenance of an expression vector into the organism, or the direct modification of the organisms genome by directly adding or deleting genes through processes like, but not limited to, 2 step allelic exchange or CRISPR cloning.

The term ribosomal binding site (RBS) refers to the region within a polynucleotide sequence that allows for the appropriate binding of a ribosome to a polynucleotide sequence to facilitate the translation of a polynucleotide sequence to produce a polypeptide sequence, which includes the terms protein, enzyme, and plasmid.

The term synthetic promoter refers to the addition or modification of a promoter sequence that would not or does not exist within the organism naturally. This can include the insertion or utilization of non-native promoters, or regions of non-native promoters utilized in the modification of protein synthesis.

The term biosynthetic in the context of the present invention refers to the generation of a biological compound by a living organism. This can include but is not limited to the formation of a biological compound that naturally occurs with the organism or the formation of a compound by an organism due to modifications to its genetic code.

The term transgenic, as used herein, refers to the combination of multiple organism polynucleotide sequences within a single organism. For example, if a polynucleotide sequence was sourced from an organism outside of the intended organism of interest within the invention, the organism of the invention's interest would be considered transgenic in nature.

The term cloning vector herein refers to a polynucleotide sequence or plasmid that can be replicated within a host organism for storage or amplification purposes. A cloning vector may contain all the necessary regulatory sequences needed to facilitate the transcription and translation of a protein.

The term unmodified promoter is defined as a promoter sequence which is unaltered and/or exists within the host organism itself.

The term two-step allelic exchange is referring to a process by which a gene of interest is either inserted or deleted from an organism through specific selective conditions. The insertion or deletion of a specific gene of interest is done so through the utilization of distinct polynucleotide sequences which allows for the exchange of genetic material between two sources.

The term CRISPR cloning is defined as a process by which the gene of interest is inserted or removed from an organism's genome using the CRISPR-CAS9 cloning system.

The terms upstream and downstream refer to regions of polynucleotides which are found prior to or after a specific gene of interest within a plasmid and/or genome of an organism.

The term enzyme within the context of the present invention defines a polypeptide sequence, specifically in the form of a protein, that can modify a biological molecule or take part within its generation through direct or indirect interactions. The process by which an enzyme influences the modification and/or production of a biological molecule and/or product is termed enzymatic activity.

The term open reading frame (ORF) refers to the collection of nucleotides which are found in between the start and stop codons of a polypeptide encoding DNA sequence.

The term codon(s) refers to 3 adjacent nucleotides in a polynucleotide sequence that are used by the cell to “decode” the polynucleotide sequence when the polynucleotide sequence is translated to make the polypeptide sequence and are responsible defining the order of protein residues in a polypeptide sequence based on this code. Based on a 3 letter code, and 4 different nucleotide bases, these codons include 64 different combinations that are able to be used by the cell, which with some redundancy codes for 22 possible protein residues, as well as 1 start and 3 stop codons.

A start and stop codon refer to nucleotide codon sequences comprised of three specific nucleotides in succession of each other, which allows for the identification of the initiation (start) and termination (stop) for the translation of a polypeptide sequence by the cell.

A unicellular organism refers to any organism of which complete organismal composition consists of a single cell.

The term central dogma of molecular biology states that genetic material flows in a single direction to produce protein. This dogma states that DNA is transcribed to produce messenger RNA, which in turn is translated to produce the final protein/polypeptide sequence. Simply put: DNA→messenger RNA→Protein

The term messenger RNA refers to a transitory molecule that is found between the polypeptide sequence and the DNA polynucleotide sequence. Simply, the messenger RNA is transcribed from the polynucleotide sequence and the messenger RNA is translated to produce the final protein.

The term metabolic engineering herein refers to the alteration of an organism's metabolic pathway potential. This can include both the deactivation and/or altering of pre-existing metabolic pathways of an organism or the inclusion of additional metabolic processes.

The term “Sequence alignment” herein refers to a bioinformatic technique by which two polynucleotide sequences or two polypeptide sequences are arranged or aligned in such a way as to identify regions of similarity between a reference sequence (the sequence that is known) and the quarry sequence (the sequence to be compared to the reference sequence). Those skilled in the art know that alignment algorithms such as, but by no means limited to, the BLAST, ALIGN, or CLUSTAL algorithms can be used to obtain this information for polynucleotide or polypeptide sequences, respectively.

The term “Percentage sequence identity” herein refers to the similarity between 2 sequences that have been processed through a sequence alignment, to provide insight into how similar aligned sequences are at either the nucleotide or peptide level for polynucleotide or polypeptide sequences, respectively. The percentage identity is used to determine the similarity of a query sequence to a reference sequence.

The term “Percentage sequence coverage” refers to the number of aligned nucleotides or peptides in a query sequence relative to the length of the reference sequence. The percentage coverage provides an indication of how much of the reference polynucleotide or polypeptide sequence is covered by the query sequence, allowing for instance the lengths of the found genes or proteins to be compared.

The BLASTN algorithm was used herein as one method to determine the percentage identity and percentage coverage between one or even multiple different polynucleotide sequences with respect to an inputted reference sequence, allowing for the determination of the percentage identity and percentage coverage of one or many query sequences to said reference sequence. One of ordinary skill in the art will recognize that search results from a BLASTN search will be influenced by the search parameters used in the search. Therefore, for all BLASTN searches done with respect to this invention to identify other sequences which have been catalogued in the NCBI polynucleotide databases relative to a reference include the following parameters:
Search set parameters are comprising of “standard databases (nr ect)”, with the specific database used being the “Nucleotide collection (nr/nt)”, and no exclusions or limitations were placed on the search (all default parameters)
Program selection algorithm parameters includes the highly similar sequences (known as the megablast algorithm) (the default parameter)
Algorithm parameters altered include the Max target sequences, which was set at 5000, otherwise all default parameters are used for relevant searches (other parameters in “General parameters”, and all parameters in “Scoring parameters” and “Filters and masking” are default parameters)

The BLASTP algorithm was used herein as one method to determine the percentage identity and percentage coverage between one or even multiple different polypeptide sequences with respect to an inputted reference sequence, allowing for the determination of the percentage identity and percentage coverage of one or many query sequences to said reference sequence. One of ordinary skill in the art will recognize that search results from a BLASTP search will be influenced by the search parameters used in the search. Therefore, for all BLASTP searches done with respect to this invention to identify other sequences which have been catalogued in the NCBI polypeptide databases relative to a reference include the following parameters:
Search set parameters are comprising of “standard databases (nr ect)”, with the specific database used being the “Non-redundant protein sequences (nr)”, and no exclusions or limitations were placed on the search (all default parameters)
Program selection algorithm parameters includes the BLASTP (known as the “protein-protein” BLAST algorithm) (the default parameter)
Algorithm parameters altered include the Max target sequences, which was set at 5000. Otherwise all default parameters are used for relevant searches (other parameters in “General parameters”, and all parameters in “Scoring parameters” and “Filters and masking” are default parameters). Notable default parameters include an “Expect Threshold and word size of 0.05 and 5, respectively in the general parameters, the usage of the BLOSUM62 matrix with gap costs of Existence: 11 and Extension: 1 for the Scoring parameters, and no filter or masking components selected.

The phrases “substantially similar” or “substantially identical” in the context of at least 2 nucleic acid sequences or at least 2 polypeptide sequences typically means that a polynucleotide, polypeptide, or region or domain of a polypeptide has, preferably, a percentage coverage of at least 70%, 75%, 80%, 85%, 90%, 95%, 97%, 99%, or even 99.5%, and at least 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% percentage identity to the reference sequence. Some polynucleotide or polypeptide sequences that fall in this category are sequences that share genetic or protein homology to the reference sequence.

The terms “genetic homology” and “protein homology”, or “homologous sequences” refer to polynucleotide sequences or translated polypeptide sequences that have a similar or identical function in the cell. For example, 2 different proteins share a similar or identical function even though they were isolated from 2 different organisms. Polynucleotide sequences with homology are generally understood to have similar or identical biochemical functionality.

In scientific literature, genes/proteins are often renamed as more about the gene is determined, often leaving several different associated names for each gene. The FMO1 protein is known as flavin-containing monooxygenase 1. The ADO protein is known as cysteamine dioxygenase and 2-aminoethanethiol dioxygenase. The VNN1 protein is known as vanin-1 and pantetheinase. The VNN2 protein is known as vanin-2 and as pantetheinase. The VNN3 protein is known as vanin-3 and as pantetheinase.

### Enzymes and Promoters

Example polypeptide sequences for enzymes involved in the synthesis of a sulfur-containing compound that can be integrated into prokaryotic organisms are provided in the sequence listing. According to a preferred embodiment of the present invention, the expression and production of these sequences within the cell are partially driven by the genetic polynucleotide promoter and ribosomal binding site sequences as provided in the sequence listings: SEQ 5 (P_{glyA}), SEQ 6 (P_{SOD}), SEQ 7 (P_{pgk}), SEQ 8 (P_{tuf}), SEQ 9 (P_{fbaA}), SEQ 10 (P_{lysC}), SEQ 11 (Pₜₖₜ), SEQ 12 (P_{glnA}), SEQ 13 (P_{pyc}), SEQ 14 (Pₕₒₘ), SEQ 15 (P_{gnd}), SEQ 16 (P_{lysA}), SEQ 17 (P_{aspB}), SEQ 18 (P_{ddh}), SEQ 19 (P_{dapB}), SEQ 20 (P_{dapA}) and SEQ 21 (P_{tac}). The invention is not limited to the use of these amino acid sequences.

Those of ordinary skill in the art know that organisms of a wide variety of species commonly express and utilize homologous proteins, which contain insertions, substitutions and deletions in the polypeptide sequences listed above, and effectively provide a similar function. For example, the protein sequences for ADO from Sus scrofa or Nycticebus coucang or Salmo salar and VNN from Sus scrofa or Nycticebus coucang or Harpia harpyja may differ to different degrees from the polypeptide sequences seen between these organisms yet maintain similar or identical functions of the protein within the organism with respect to regulatory or catalytic function. Protein sequences comprising such variations are included within the scope of the present invention and are considered substantially or sufficiently similar to the reference polypeptide sequences provided above. Although it is not intended that the present invention is limited by any theory by which it achieves its advantageous result, it is believed and supported by biochemical knowledge that the identity between polypeptide sequences that is necessary to maintain proper functionality is related to maintaining the tertiary (3D) structure of the polypeptide. This maintenance of the tertiary structure is associated with the specific interactive/catalytic portions of the protein sequence and will therefore have the desired activity, and it is contemplated that a protein including these interactive sequences in the proper spatial context will have this activity.

Those of ordinary skill in the art know that many different amino acids contain similar properties between each other and can serve similar functions in the final polypeptide sequence. Thus, when one amino acid is changed with another amino acid from this group, such as a non-polar amino acid, an uncharged polar amino acid, a charged polar acidic amino acid, or a charged polar basic amino acid, some polypeptide functionality is generally maintained. For example, it is known that the uncharged polar amino acid serine may be substituted for the uncharged polar amino acid threonine in a polypeptide without substantially altering the protein structure and functionality. Whether a given substitution will affect the functionality of the enzyme may be determined without undue experimentation using synthetic techniques and screening assays known to a person of ordinary skill in the art.

A person of ordinary skill in the art will recognize that changes in the protein sequence, resulting from individual single or multi-nucleotide substitutions, deletions, or additions to a polynucleotide will lead to changes in the resulting translated polypeptide sequence. Small mutations, such as the change of an amino acid from one to another, or the addition or elimination of single amino acids, or a small to moderate percentage of amino acids from the encoded polypeptide sequence can be considered “sufficiently similar” when the alteration results in the substitutions of an amino acid with a chemically similar amino acid. Thus, any number of amino acid residues in a polypeptide chain, selected from a group of integers from 1-50, can be so altered. Thus, for example, 1, 2, 3, 5, 10, 12, 20, 32, 41, or even 50 alterations can be made. Conservatively modified variants typically provide similar biological activity as the unmodified polypeptide sequence from which they are derived. For example, modification of ADO and VNN to yield functional proteins generally have, preferably, a sequence identity of at least 40%, 50%, 60%, 70%, 80%, or 90%, preferably a sequence identity of greater than 50%, of the native protein to allow processing of its native substrate. Tables of conserved substitution provide lists of functionally similar amino acids. Amino acids in polypeptide chains that are similar to one another include, but are not limited to, the following groups: (1) Serine (S), Threonine (T); (2) Aspartic acid (D), Glutamic acid (E); (3) Asparagine (N), Glutamine (Q); (4) Alanine (A), Leucine (L), and Isoleucine (I).

### Suitable Polynucleotide and Polypeptide Sequences for ADO and VNN

A person of ordinary skills in the art will recognize that many different organisms will have functionally similar polynucleotide and polypeptide sequences (or homology between the sequences), however there may be differences between these sequences when compared to a reference sequence. As examples, suitable polynucleotides and their corresponding polypeptide sequences for the production of a sulfur-containing compound can be seen below. Note that the following sequences by no means are meant to limit the scope of the invention. In fact, any substantially similar polynucleotide sequences or substantially similar produced polypeptide sequences for the ADO and VNN genes with similar function or similarity to these genes can also be used for the production of a sulfur-containing compound.

According to a preferred embodiment of the present invention, the polynucleotide sequence for ado, isolated from the eukaryotic species Sus scrofa (pig) (SEQ 1), was utilized in the process described herein. In this embodiment, cysteamine dioxygenase (ado) is under the transcriptional control of a native or artificial promoter and a ribosomal binding site. However, in other embodiments of the invention, polynucleotide sequences that are homologous and/or substantially similar to SEQ 1 may also be used in the present invention to produce taurine. Polynucleotide sequences for cysteamine dioxygenase in these embodiments will, preferably, have at least 70% sequence coverage, or more preferably greater than 80%. 90%, 95%, 98%, or most preferentially greater than 99% sequence coverage of SEQ 1, and sequence identities of at least 70%, or more preferentially greater than 80%, 90%, 95%, 97% sequence identity, and most preferentially 99% sequence identity of SEQ 1. These polynucleotide sequences may include, but by no means limited to, the following sequences: SEQ 22. SEQ 23, SEQ 24, SEQ 25, SEQ 26, and SEQ 27.

According to a preferred embodiment of the present invention, the cysteamine dioxygenase polypeptide (ADO) SEQ 2 from the eukaryotic species Sus scrofa (pig) is utilized, whereby SEQ 2 is produced from the transcription and translation of the cysteamine dioxygenase polynucleotide SEQ 1. However, in other embodiments of the invention, polypeptide sequences that are homologous and/or substantially similar to SEQ 2 may also be used in the present invention to produce taurine. Polypeptide sequences for cysteamine dioxygenase in these embodiments will, preferably, have at least 70% sequence coverage, or more preferentially greater than 80%, 90%, 95%, 98%, or most preferentially greater than 99% sequence coverage of SEQ 2, and a sequence identity of, preferably, at least 25% to SEQ 2, or more preferentially greater than 30%, 35%, 40%, 45%, 50%, 55%, 60%, 65%, 70% 75%, 80%, 85%, 90%, 95%, 97%, or most preferentially greater than 99% sequence identity to SEQ 2. These polypeptide sequences may include, but are not limited to, the following sequences: SEQ 28, SEQ 29, SEQ 30, SEQ 31, SEQ 32, SEQ 33, SEQ 34, SEQ 35, SEQ 36, SEQ 37, SEQ 38, SEQ 39, SEQ 40, SEQ 41, and SEQ 42.

According to a preferred embodiment of the present invention, the polynucleotide sequence for vanin-1 (vnn1), isolated from the eukaryotic species Sus scrofa (pig) SEQ 3, was utilized. However, in other embodiments of the invention, polynucleotide sequences that are homologous and/or substantially similar to SEQ 3 may also be used in the present invention. Furthermore, polynucleotide sequences that are homologous and/or substantially similar to SEQ 69 may also be used in the present invention. Polynucleotide sequences for vanin-1 in these embodiments will have at least 70% sequence coverage, or more preferentially greater than 75%, 80%, 85%, 90%, 95%, 97%, or 98% sequence coverage, or most preferentially greater than 99% sequence coverage of SEQ 3 or SEQ 69, and the polynucleotide sequence of vanin-1 has at least 70% sequence identity, or more preferentially 80%, 85%, 90%, 95%, 97%, or 98% sequence identity, or most preferentially greater than 99% sequence identity to SEQ 3 or SEQ 69. These polynucleotide sequences may include, but by no means are limited to, the following sequences: SEQ 43, SEQ 44, SEQ 45, SEQ 46, SEQ 47, SEQ 48, SEQ 49. SEQ 50, SEQ 51, and SEQ 52.

According to a preferred embodiment of the present invention, the vanin-1 polypeptide (VNN1) SEQ 4 from the eukaryotic species Sus scrofa (pig) is utilized to produce a sulfur-containing compound by the cell, whereby SEQ 4 is produced from the transcription and translation of the vanin-1 polynucleotides SEQ 3 or SEQ 69. However, in other embodiments of the invention, polypeptide sequences that are homologous and/or substantially similar to SEQ 4 may also be used in the present invention to produce taurine. Polypeptide sequences for vanin-1 in these embodiments will, preferably, have at least 70% sequence coverage, or more preferentially greater than 80%, 90%, 95%, 98%, or most preferentially greater than 99% sequence coverage of SEQ 4, and a sequence identity of at least 25% to SEQ 4, or more preferentially greater than 30%, 35%, 40%, 45%, 50%, 55%, 60%, 65%, 70% 75%, 80%, 85%, 90%, 95%, 97%, or most preferentially greater than 99% sequence identity to SEQ 4. These polypeptide sequences may include, but are by no means limited to, the following sequences: SEQ 53. SEQ 54, SEQ. 56, SEQ 57, SEQ 58, SEQ 59, SEQ 60, SEQ 61, SEQ 62, SEQ 63, SEQ 64, SEQ, 65, SEQ 66, SEQ 67, and SEQ 68.

According to a preferred embodiment of the present invention, the polynucleotide sequence for vanin-2 (vnn2), isolated from the eukaryotic species Bos taurus (cattle) SEQ 70 can be utilized in the place of vanin-1 (vnn1) (SEQ 3 or SEQ 69). However, in other embodiments of the invention, polynucleotide sequences that are homologous and/or substantially similar to SEQ 70 may also be used in the present invention to produce taurine. Polynucleotide sequences for vanin-2 in these embodiments will have at least 70% sequence coverage, or more preferentially greater than 80%, 85%, 90%, 95%, 96%, or 97% sequence coverage, or most preferentially greater than 99% sequence coverage of SEQ 70, and the polynucleotide sequence of vanin-2 has at least 70% sequence identity, or more preferentially 80%, 85%, 90%, 95%, or 96% sequence identity, or most preferentially greater than 99% sequence identity to SEQ 70. These polynucleotide sequences may include, but by no means are limited to, the following sequences: SEQ 71; SEQ 72; SEQ 73; SEQ 74; SEQ 75; SEQ 76; SEQ 77; SEQ 78; SEQ 79; SEQ 80; SEQ 81; SEQ 82; and SEQ 83.

According to a preferred embodiment of the present invention, the vanin-2 polypeptide (VNN2) SEQ 84 from the eukaryotic species Bos taurus (cattle) is utilized, whereby SEQ 84 is produced from the transcription and translation of the vanin-2 polynucleotide SEQ 70. However, in other embodiments of the invention, polypeptide sequences that are homologous and/or substantially similar to SEQ 84 may also be used in the present invention to produce taurine. Polypeptide sequences for vanin-2 in these embodiments will, preferably, have at least 70% sequence coverage, or more preferentially greater than 75%, 80%, 90%, 95%, or most preferentially greater than 99% sequence coverage of SEQ 84, and a sequence identity of at least 25% to SEQ 84, or more preferentially greater than 30%, 35%, 40%, 45%, 50%, 55%, 60%, 65%, 70% 75%, 80%, 85%, 90%, 95%, 98%, or most preferentially greater than 99% sequence identity to SEQ 84. These polypeptide sequences may include, but are by no means limited to, the following sequences: SEQ 85; SEQ 86; SEQ 87; SEQ 88; SEQ 89; SEQ 90; SEQ 91; SEQ 92; SEQ 93; SEQ 94; SEQ 95; SEQ 96; SEQ 97; SEQ 98; SEQ 99; SEQ 100; SEQ 101; SEQ 102; SEQ 103; SEQ 104; SEQ 105; SEQ 106; SEQ 107; SEQ 108; SEQ 109; and SEQ 110.

According to a preferred embodiment of the present invention, the polynucleotide sequence for vanin-3 (vnn3), isolated from the eukaryotic species Mus musculus (house mouse) (SEQ 111) can be utilized in the place of vanin-1 (vnn1) (SEQ 3 or SEQ 69). However, in other embodiments of the invention, polynucleotide sequences that are homologous and/or substantially similar to SEQ 111 may also be used in the present invention to produce taurine. Polynucleotide sequences for vanin-3 in these embodiments will have at least 70% sequence coverage, or more preferentially greater than 75%, 80%, 85%, 90%, 95%, 96%, or 97% sequence coverage, or most preferentially greater than 99% sequence coverage of SEQ 111, and the polynucleotide sequence of vanin-3 has at least 70% sequence identity, or more preferentially 75%. 80%, 90%, 95%, or 97% sequence identity, or most preferentially greater than 99% sequence identity to SEQ 111. These polynucleotide sequences may include, but by no means are limited to, the following sequences: SEQ 112; SEQ 113; SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; and SEQ 127.

According to a preferred embodiment of the present invention, the vanin-3 polypeptide (VNN3) SEQ 128 from the eukaryotic species Mus musculus (house mouse) is utilized to produce a sulfur-containing compound by the cell, whereby SEQ 128 is produced from the transcription and translation of the vanin-3 polynucleotide SEQ 111. However, in other embodiments of the invention, polypeptide sequences that are homologous and/or substantially similar to SEQ 128 may also be used in the present invention to produce taurine. Polypeptide sequences for vanin-3 in these embodiments will, preferably, have at least 70% sequence coverage, or more preferentially greater than 75%, 80%, 85%, 90%, 95%, 96%, 97%, 98%, or most preferentially greater than 99% sequence coverage of SEQ 128, and a sequence identity of at least 25% to SEQ 128, or more preferentially greater than 30%, 35%, 40%, 45%, 50%, 55%, 60%, 65%, 70% 75%, 80%, 85%, 90%, 95%, 98%, or most preferentially greater than 99% sequence identity to SEQ 128. These polypeptide sequences may include, but are by no means limited to, the following sequences: SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; SEQ 146; SEQ 147; SEQ 148; SEQ 149; SEQ 150; SEQ 151; SEQ 152; and SEQ 153.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A genetically modified prokaryotic cell which comprises a cysteamine dioxygenase (ado) polynucleotide sequence which has at least 70% sequence coverage to SEQ 1, and at least 70% sequence identity to SEQ 1.

2. The genetically modified prokaryotic cell according to claim 1, wherein said ado polynucleotide sequence is selected from the group consisting of: SEQ 1; SEQ 22; SEQ 23; SEQ 24; SEQ 25; SEQ 26; and SEQ 27.

3. The genetically modified prokaryotic cell according to claim 1, wherein upon transcription and translation under the control of a native or synthetic promoter and Ribosomal binding site (RBS), said ado polynucleotide sequences provides a cysteamine dioxygenase (ADO) polypeptide sequence which has at least 70% sequence coverage to SEQ 2, and at least 25% sequence identity to SEQ 2.

4. The genetically modified prokaryotic cell according to claim 3, wherein said ADO polypeptide sequence is selected from the group consisting of: SEQ 2; SEQ 28; SEQ 29; SEQ 30; SEQ 31; SEQ 32; SEQ 33; SEQ 34; SEQ 35; SEQ 36; SEQ 37; SEQ 38; SEQ 39; SEQ 40; SEQ 41; and SEQ 42.

5. A genetically modified prokaryotic cell which comprises a vanin (vnn) polynucleotide sequence selected from the group consisting of:
a vanin-1 (vnn1) polynucleotide sequence which has at least 70% sequence coverage to SEQ 3 or SEQ 69, and at least 70% sequence identity to SEQ 3 or SEQ 69;
a vanin-2 (vnn2) polynucleotide sequence which has least 70% sequence coverage to SEQ 70, and at least 70% sequence identity to SEQ 70; and
a vanin-3 (vnn3) polynucleotide sequence which has least 70% sequence coverage to SEQ 111, and at least 70% sequence identity to SEQ 111.

6. The genetically modified prokaryotic cell according to claim 5, wherein:
said vanin-1 (vnn1) polynucleotide sequence is selected from the group consisting of: SEQ 3; SEQ 43; SEQ 44; SEQ 45; SEQ 46; SEQ 47; SEQ 48; SEQ 49; SEQ 50; SEQ 51; SEQ 52; and SEQ 69; or
said vanin-2 (vnn2) polynucleotide sequence is selected from the group consisting of: SEQ 70; SEQ 71; SEQ 72; SEQ 73; SEQ 74; SEQ 75; SEQ 76; SEQ 77; SEQ 78; SEQ 79; SEQ 80; SEQ 81; SEQ 82; SEQ 83; and
said vanin-3 (vnn3) polynucleotide sequence is selected from the group consisting of: SEQ 111; SEQ 112; SEQ 113; SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; and SEQ 127.

7. The genetically modified prokaryotic cell according to claim 5, wherein upon transcription and translation under the control of a native or synthetic promoter and Ribosomal binding site (RBS), said vnn1 polynucleotide sequences provides a vanin-1 (VNN1) polypeptide sequence which has at least 70% sequence coverage to SEQ 4, and at least 25% sequence identity to SEQ 4.

8. The genetically modified prokaryotic cell according to claim 5, wherein upon transcription and translation under the control of a native or synthetic promoter and Ribosomal binding site (RBS), said vnn2 polynucleotide sequences provides a vanin-2 (VNN2) polypeptide sequence which has at least 70% sequence coverage to SEQ 84, and at least 25% sequence identity to SEQ 84.

9. The genetically modified prokaryotic cell according to claim 5, wherein upon transcription and translation under the control of a native or synthetic promoter and Ribosomal binding site (RBS), said vnn3 polynucleotide sequences provides a vanin-3 (VNN3) polypeptide sequence which has at least 70% sequence coverage to SEQ 128, and at least 25% sequence identity to SEQ 128.

10. The genetically modified prokaryotic cell according to claim 7, wherein said VNN1 polypeptide sequence is selected from the group consisting of: SEQ 4; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; and SEQ 68.

11. The genetically modified prokaryotic cell according to claim 8, wherein said VNN2 polypeptide sequence is selected from the group consisting of: SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; SEQ 97; SEQ 98; SEQ 99; SEQ 100; SEQ 101; SEQ 102; SEQ 103; SEQ 104; SEQ 105; SEQ 106; SEQ 107; SEQ 108; SEQ 109; and SEQ 110.

12. The genetically modified prokaryotic cell according to claim 9, wherein said VNN3 polypeptide sequence is selected from the group consisting of: SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; SEQ 146; SEQ 147; SEQ 148; SEQ 149; SEQ 150; SEQ 151; SEQ 152; and SEQ 153.

13. The genetically modified prokaryotic cell according to claim 1 further comprising a promoter and RBS sequence which drives gene expression, wherein the genetic material for the promoter/RBS sequences comprises at least one or another of the following: SEQ 5; SEQ 6; SEQ 7; SEQ 8; SEQ 9; SEQ 10; SEQ 11; SEQ 12;
SEQ 13; SEQ 14; SEQ 15; SEQ 16; SEQ 17; SEQ 18; SEQ 19; SEQ 20; and SEQ 21.

14. A genetically modified prokaryotic cell which comprises:
a cysteamine dioxygenase (ado) polynucleotide sequence which has at least 70% sequence coverage to SEQ 1, and at least 70% sequence identity to SEQ 1; and
a vanin (vnn) polynucleotide sequence selected from the group consisting of:
a vanin-1 (vnn1) polynucleotide sequence which has at least 70% sequence coverage to SEQ 3 or SEQ 69, and at least 70% sequence identity to SEQ 3 or SEQ 69;
a vanin-2 (vnn2) polynucleotide sequence which has at least 70% sequence coverage to SEQ 70, and at least 70% sequence identity to SEQ 70; and
a vanin-3 (vnn3) polynucleotide sequence which has at least 70% sequence coverage to SEQ 111, and at least 70% sequence identity to SEQ 111.

15. The genetically modified prokaryotic cell according to claim 14, wherein:
said ado polynucleotide sequence is selected from the group consisting of: SEQ 1; SEQ 22; SEQ 23; SEQ 24; SEQ 25; SEQ 26; and SEQ 27.
said vanin-1 (vnn1) polynucleotide sequence is selected from the group consisting of: SEQ 3; SEQ 43; SEQ 44; SEQ 45; SEQ 46; SEQ 47; SEQ 48; SEQ 49; SEQ 50; SEQ 51; SEQ 52; and SEQ 69;
said vanin-2 (vnn2) polynucleotide sequence is selected from the group consisting of: SEQ 70; SEQ 71; SEQ 72; SEQ 73; SEQ 74; SEQ 75; SEQ 76; SEQ 77; SEQ 78; SEQ 79; SEQ 80; SEQ 81; SEQ 82; SEQ 83; and
said vanin-3 (vnn3) polynucleotide sequence is selected from the group consisting of: SEQ 111; SEQ 112; SEQ 113; SEQ 114; SEQ 115; SEQ 116; SEQ 117; SEQ 118; SEQ 119; SEQ 120; SEQ 121; SEQ 122; SEQ 123; SEQ 124; SEQ 125; SEQ 126; and SEQ 127.

16. A genetically modified prokaryotic cell which comprises:
a cysteamine dioxygenase (ADO) polypeptide sequence which has at least 70% sequence coverage to SEQ 2, and at least 25% sequence identity to SEQ 2; and
a vanin (VNN) polypeptide sequence selected from the group consisting of:
a vanin-1 (VNN1) polypeptide sequence which has at least 70% sequence coverage to SEQ 4, and at least 25% sequence identity to SEQ 4;
a vanin-2 (VNN2) polypeptide sequence which has at least 70% sequence coverage to SEQ 84, and at least 25% sequence identity to SEQ 84; and
a vanin-3 (VNN3) polypeptide sequence which has at least 70% sequence coverage to SEQ 128 and at least 25% sequence identity to SEQ 128.

17. The genetically modified prokaryotic cell according to claim 16, wherein:
said ADO polypeptide sequence is selected from the group consisting of: SEQ 1; SEQ 22; SEQ 23; SEQ 24; SEQ 25; SEQ 26; and SEQ 27;
said vanin-1 (VNN1) polypeptide sequence is selected from the group consisting of: SEQ 4; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; SEQ 67; and SEQ 68;
said vanin-2 (VNN2) polypeptide sequence is selected from the group consisting of: SEQ 84; SEQ 85; SEQ 86; SEQ 87; SEQ 88; SEQ 89; SEQ 90; SEQ 91; SEQ 92; SEQ 93; SEQ 94; SEQ 95; SEQ 96; SEQ 97; SEQ 98; SEQ 99; SEQ 100; SEQ 101; SEQ 102; SEQ 103; SEQ 104; SEQ 105; SEQ 106; SEQ 107; SEQ 108; SEQ 109; and SEQ 110; and
said vanin-3 (VNN3) polypeptide sequence is selected from the group consisting of: SEQ 128; SEQ 129; SEQ 130; SEQ 131; SEQ 132; SEQ 133; SEQ 134; SEQ 135; SEQ 136; SEQ 137; SEQ 138; SEQ 139; SEQ 140; SEQ 141; SEQ 142; SEQ 143; SEQ 144; SEQ 145; SEQ 146; SEQ 147; SEQ 148; SEQ 149; SEQ 150; SEQ 151; SEQ 152; and SEQ 153.

18. The prokaryotic cell according to claim 1, where the cell is a bacterial cell.

19. The prokaryotic cell according to claim 1, where the cell is selected from the group consisting of the genera: Brevibacterium, Bacillus, Corynebacterium, Escherichia, Lactococcus, Pseudomonas, Rhodococcus, and Serratia.

20. The prokaryotic cell according to claim 1, where the cell belongs to the genus Corynebacterium.


SEQUENCE LISTING (153 sequences)

SEQ ID NO: 1 - DNA, 813 bp, Sus scrofa
  source 1..813: mol_type = genomic DNA
atgccccgagacaacatggcctccctgatccaacggatcgcccgccaggcatgcctcaccttccggggcagcgggggcggccgcagctcttccgatcgcggcgcggcgccaggccctgaggcgcctgtgccgcagggcttcccggagaacctgagcaagctgaagagcctgctgacacaggtccgcgcagaggacctgaacatctccccgcgcaaggccacgctgcagccgttgccacccaacctgccgcccgtcacctatatgcacatctacgagactgacggcttcagcctcggcgtgttcttgcttaagagcggcacatccatcccgctccacgaccaccctggcatgcatggcatgctcaaggtgctctatggcaccgtgcgcatcagctgcatggacaagctggaggcaggcagcgggcaacggccgcgggccccgccaccagagcagcagttcgaaccgccgctgctggcccgggagcgggacgcggtgcggccgggagtgctgcgctcgcgggccgagtacactgaggccagcggtccctgcgtcctcacgccgcaccgggacaacctgcaccagatcgacgctgtggatgggcctgccgccttcttggatatcctggccccgccctacgacccggacgacggccgggactgtcactattaccgggtgctggagcctgtcagggccaaagaggcctccgactcggcctgtgacctgccccgagaggtgtggcttctggagaccccgcaggccgatgacttttggtgcgagggggagccctatccaggtcccagggtcttcccttga

SEQ ID NO: 2 - protein, 270 aa, Sus scrofa
MPRDNMASLIQRIARQACLTFRGSGGGRSSSDRGAAPGPEAPVPQGFPENLSKLKSLLTQVRAEDLNISPRKATLQPLPPNLPPVTYMHIYETDGFSLGVFLLKSGTSIPLHDHPGMHGMLKVLYGTVRISCMDKLEAGSGQRPRAPPPEQQFEPPLLARERDAVRPGVLRSRAEYTEASGPCVLTPHRDNLHQIDAVDGPAAFLDILAPPYDPDDGRDCHYYRVLEPVRAKEASDSACDLPREVWLLETPQADDFWCEGEPYPGPRVFP

SEQ ID NO: 3 - DNA, 1582 bp, Sus scrofa
  source 1..1582: mol_type = genomic DNA
atgatcacatctccgttgctggcttatgtggcgattctgtttttgtgtgtcttaaaagccagctccctggatacctttatcgccgctgtttatgaacatgcagtgatattgcctgatgcccccctcacaccagtatcccatgaagaggctttgatgttaatgaaccgcaatctggatcttttggagggcgcaatcatgtcagcagcaaagcagggtgcacatattattgtgactccagaagatggtgtttacggctttttcttcagcagggaatctatttactcatacttagaggacatcccagagcctcacgtgaactggatcccttgtaccaatcccaacagatttggccacactccagtacaaaaaagactcagctgcctggccagggacaactccatctacattgtggcaaatattggggacaagaagccatgcaatgccagtgaccccgactgtcctcatgacggccgttaccagtacaacactgatgtggtgtttgattctgagggcagactggtggcacgctatcataagcaaaatcttttcttgggtgaagatcaattcgatgcacccaaggaacccgagatcgtgactttcgacaccacctttggaagatttggcattttcacgtgctttgatattctcttccatgatcctgctgtgactctggtgaaagatttccaggtggacaccatactcttcccgacagcttggatgaatgttttgccacatttgacagctattgaattccactcagcttgggctatgggcatgagagtcaacttccttgcagccaatatacatttccccttaagaaaaatgacaggaagtggcatctatgcacctgattctccaagagcatttcattacgatatgaagacaaaggagggaaaactcctcctctcacaattggactcccacccacaccgcccagcagtgaattggacttcttatgccagtggtttaccagcccccttagtgggaaaccaggaatttaaaagcattgtcttttttgacgagttcaccttcttcgagctcaaaggagttgcaggaaattacacagtttgtcagaaagatctctgctgtcagctaagttacaggatgcttgagatgagagaagacgaagtttatgcactaggggcattcgatggactacatactgtggaagggagctattatttacagatttgtactttgttgaagtgtaaaacaatggacttacactcctgtggtgactcggtggaaactgcttccaccaggtttgaaatgttctccctcagtggcacttttggaacccagtatgtgttccctgaggtgttgctgagtgacatacagcttgcacctggagaatttcaggtgtcaagtgatggacgcttgttcagcctgaagccaccgtctggacctgtcttgactgtcactctgtttgggaggttatatgagagggatagtgcatcaggtgcttcagcagacctcgtggcacaaggactgagagtcatgctcggagttataataactattatgtactcattaagttggtagaatatttatacttttctctctttcacttcggatcctttaa

SEQ ID NO: 4 - protein, 513 aa, Sus scrofa
MITSPLLAYVAILFFCVLKASSLDTFIAAVYEHAAILPDAPLTPVSHEEALMLMNRNLDLLEGAVTSAAKQGAHIIVTPEDGVYGFFFSRESIYSYLEDIPDPHVNWIPCTNPSRFGHTPVQKRLSCLARDNSIYIVANIGDKKPCNASDPDCPHDGRYQYNTDVVFDSEGRLVARYHKQNLFLGEDQFDAPKEPEIVTFDTTFGRFGIFTCFGILFHDPAVTLVKDFQVDTILFPTAWMNVLPHLTAIEFHSAWAMGMRVNFLAANIHFPLRKMTGSGIYAPDSPRAFHYDMKTKEGKLLLSQLDSHPHRPAVNWTSYASGLPTPLVGNQEFKSTVFFDEFTFLELKGVAGNYTVCQKDLCCQLSYRMLEKREDEVYALGAFDGLHTVEGSYYLQICTLLKCKTMDLHSCGDSVETASTRFEMFSLSGTFGTQYVFPEVLLSDIQLAPGEFQVSSDGRLFSLKPPSGPVLTVTLFGRLYERDSASGASADLVAQGLRVMLGVIITIMYSLSW

SEQ ID NO: 5 - DNA, 251 bp, Corynebacterium glutamicum
  source 1..251: mol_type = genomic DNA
cttgcagcgttttgctctgccatgaatccattgtgcaccttagctactccattagtgtgatcggggttattttttcacttcaatgggtggctaaaagacgtgggcacgtgagtaaactcatgcgcgcgaaacgatggaagtgaacccatacttttatatatgggtatcggcggtctatgcttgtgggcgtacctgtcccgcgagtgaggtcttacgcgcgggattcgtcttgtgaaaggttagctgacctg

SEQ ID NO: 6 - DNA, 250 bp, Corynebacterium glutamicum
  source 1..250: mol_type = genomic DNA
gccataacaggaatgttcctttcaaaaattgaggaagccttatgacctacaaccctactcagctgccgattattccgggtttgtgaccagctacccgataaataggtcggctgaaaaatttcgttgcaatatcaacaaaaaggcctatcattgggaagtgtcgcaccaagtacttttgcgaagcgccatctgacggattttcaaaagatgtatatgctcggtgcggaaacctacgaaaggatttttaccc

SEQ ID NO: 7 - DNA, 250 bp, Corynebacterium glutamicum
  source 1..250: mol_type = genomic DNA
gcgtctgaccgagctcgtagcttccaagctctaattagttcacatcgctaacgtgggcgatcgatgctcacggtgatgtgtcatcccaatagcccggggtgtgcctcggcgcaccccgggctattttgtgtctttaatcaatacaattgaataccggtgccagcgccacacaatgtgtggcaatctgggacagtgcatcacattgcaccagaagaattttttaaacaatcaaatctccaaggagtacggc

SEQ ID NO: 8 - DNA, 250 bp, Corynebacterium glutamicum
  source 1..250: mol_type = genomic DNA
tagatatcttgaaatcggctttcaacagcattgatttcgatgtatttagctggccgttaccctgcgaatgtccacagggtagctggtagtttgaaaatcaacgccgttgcccttaggattcagtaactggcacattttgtaatgcgctagatctgtgtgctcagtcttccaggctgctgatcacagtgaaagcaaaaccaattcgtggctgcgaaagtcgtagccaccacgaagtccaggaggacataca

SEQ ID NO: 9 - DNA, 250 bp, Corynebacterium glutamicum
  source 1..250: mol_type = genomic DNA
tagccccgatagtgtatgtgctgacctgcgtttatgcattttccttaggttgaaggaaatatcacacgacaaaagttgagtgatgcaggcataattggctatgggcaactgaagatgccacaatcaatgtttgatcaaacagaatcagacattgtgaatgtgggattatcggttcgcgcttcaccatgtttctgcatgatgaaattacatacatagttcagtgacagtcaccttttggaggagacacctt

SEQ ID NO: 10 - DNA, 250 bp, Corynebacterium glutamicum
  source 1..250: mol_type = genomic DNA
gcacttgattcagggtagttgactaaagagttgctcgcgaagtagcacctgtcacttttgccacaaataccaaattgcttatcagcatatgggctgtttgcttgccccagtcccagcagctgagacgcatccgctaaagccccaggaaccctgtacagaaagaaaacactcctctggctaggtagacacagtttataaaggtagagttgagcgggtaactgtcagcacgtagatcgaaaggtgcacaaag

SEQ ID NO: 11 - DNA, 250 bp, Corynebacterium glutamicum
  source 1..250: mol_type = genomic DNA
cacgtcacagctcccagacttaagattggtcacacctttgacacatttggaccacagttggttataaaatgggttcaacatcactatggttagaggtgttgacgggtcagattaagcaaagactactttcggggtagatcacctttgccaaatttgaaccaattaacctaagtcgtagatctgatcatcggatctaacgaaaacgaaccaaaactttggtcccggtttaacccaggaaggattgaccacc

SEQ ID NO: 12 - DNA, 250 bp, Corynebacterium glutamicum
  source 1..250: mol_type = genomic DNA
gcggatactgcagtcataggcgttgcttttcgacgtcgcaaagcgcaatttcctacctttaagatcctaatctgttgaggtcagccacaatttttcagaaaagttttgatagatcgacaggtaatgctttatactgacaacgtcgcaaggactacatttgcagccaagtctactacttgatcttcaaaggtcagcaattgtgaacaaagctacaaataaaccgttccgcccatgtcaatgaggagtcacc

SEQ ID NO: 13 - DNA, 250 bp, Corynebacterium glutamicum; identical to SEQ ID NO: 12
  source 1..250: mol_type = genomic DNA

SEQ ID NO: 14 - DNA, 250 bp, Corynebacterium glutamicum
  source 1..250: mol_type = genomic DNA
gagcgagcgaagtttcaaaatgccttcctgggggtgaaattagatcctgttaatgtactaaatttttcaaaagttttctcgttgaaaattaagaagctgggaaggtgaattgaattttgaggctttaaagcaaaaatgaacagcttggtctatagtggctaggtaccctttttgttttggacacctgtagggtggccgaagcaatgaaataggacaacaacgctcgaccgcgattattttttggagaatc

SEQ ID NO: 15 - DNA, 251 bp, Corynebacterium glutamicum
  source 1..251: mol_type = genomic DNA
ccgtaattggcgttgttgagtgcttcaagttcgtcagttgttaaagctctggtggcggcaagttctgcaagcgaaagcagatcttggggttgatcatcgcgggaagtcatattttattactctagtcggcctaaaatggttggattttcacctgctgtgacctggtaaaatggccactacccccaaatggtcacaccttttaggccgattttgctgacaccgggctatgccgtcaagtacgatcaataaca

SEQ ID NO: 16 - DNA, 250 bp, Corynebacterium glutamicum
  source 1..250: mol_type = genomic DNA
gttttcttgcgctgctgcagtgggcattgataccaaaaaggggctaagcgcagtcgaggcggcaagaactgctactaccctttttattgtcgaacggggcattacggctccaaggacgtttgttttctgggttagttaccccaaaaagcatatacagagaccaatgatttttcattaaaaaggcagggatttgttataagtatgggtcgtattctgtgcgacgggtgtacctcggctagaatttctcccc

SEQ ID NO: 17 - DNA, 250 bp, Corynebacterium glutamicum
  source 1..250: mol_type = genomic DNA
tcgtttagaaactcaaatccgctcgcagttggcgttttctggggcggttcagctagagttatgcgaaggatcccgtgcggcgtttatcttgtgaactcccccagggcaggaatgcagcaagggtcagcgagctctgacgggtgcgcgggatcccctaaaacgtctagagtagtggcttgaggtcactgctctttttttgtgcccttttttggtccgtctattttgccaccacatgcggaggtacgcagtt

SEQ ID NO: 18 - DNA, 250 bp, Corynebacterium glutamicum
  source 1..250: mol_type = genomic DNA
catccggataaaccaccatgaacggcagtggatcaaaagtcctgttggtgaagctgcgccccacagatcctgactgctgggagccatgaaaatagatcagcgcatccgtggtggaaccaaaaggctcaacaatacgaaacgttcgctttcggtcctgatgaaagagatgtccctgaatcatcatctaagtatgcatctcggtaagctcgaccaggacagtgccaccacaattttggaggattacaagaac

SEQ ID NO: 19 - DNA, 250 bp, Corynebacterium glutamicum
  source 1..250: mol_type = genomic DNA
aagaggcggaaaccacaagcgccaaggaactacctgcggaacgggctgtgaagggcaacttaagtctcatatttcaaacatagttccacctgtgtgattaatccctagaacggaacaaactgatgaacaatcgttaacaacacagaccaaaacggtcagttaggtatggatatcagcaccttctgaacgggtacgtctagactggtgggcgtttgaaaaactcttcgccccacgaaaatgaaggagcata

SEQ ID NO: 20 - DNA, 201 bp, Corynebacterium glutamicum
  source 1..201: mol_type = genomic DNA
cgcaaagctcacacccacgagctaaaaattcatatagttaagacaatatttttggctgtaaaagacagccgtaaaaacctcttgctcatgtcaattgttcttatcggaatgtggcttgggcgattgttatgcaaaagttgttaggttttttgcggggttgtttaacccccaaatgagggaagaaggtaaccttgaactcta

SEQ ID NO: 21 - DNA, 29 bp, Corynebacterium glutamicum
  source 1..29: mol_type = other DNA
ttgacaattaatcatcggctcgtataatg

SEQ ID NO: 22 - DNA, 812 bp, Balaenoptera musculus
  source 1..812: mol_type = genomic DNA
atgccccgagacaacatggcctccctgatccaacggatagcccgccaggcgtgcctcaccttccggggcagcgggggcagccgcagctcttccgaccgcggcgaggcgccaggccccgaggcgccgatgtcgcagggcttcccggagaacctgagcaagctgaagagcctgctgaccctggtccgcgcggaggacctgaacatctccccgcgcaaggccacgctgcagccgctgaggcccaacctgccgccagtcacctatatgcacatctgcgagacagacggcttcagcctcggcgtgttcctgctgaagagcggcacctccatcccgctgcacgaccacccgggcatgcacggcatcctcaaggtgctctacggcaccgtgcgcatcagctgcatggacaagctggaggtgggcagcgggcaacggccgcgggccccgccacccgagcagcagttcgagccggcgttgcagcccggggagcgggacgcggtaaggccgggtgtgctgcgctcgcgggcggagtacacggcggccagcggcccctgcgtcctcacgccgcaccgggacaacctgcaccagatcgacgctgtggaggggcccgccgctttcctggacatcctggccccgccctacgaccccgacgacggccgggactgccactattaccgggtgctggagcctgtcagggccagggaggcctccggctcggcctgtgacctgccccgagaggtgtggctcctggagaccccgcaggccgatgacttctggtgcgagggggagccctatccaggtcccaaggtcttcccttg

SEQ ID NO: 23 - DNA, 798 bp, Molossus molossus
  source 1..798: mol_type = genomic DNA
atggcctccctgatccaacggatcgcccgccaggcgtgcctcaccttccgaggcagcgggggcggccgcagtgcttccgaccgcggctcggtgccaggccccgaggcgccggggccgcagggcttcccggagaacctgagcaagctgaagagtctgctgacccaggtccgtgctgaggacctgaatatcgccccgcgcaaagcgacgctgcagccgctgccacccaacctgccacccgttacctacatgcacatctacgagacggacggcttcagcctcggcgtgttcctgctcaagagcggcacgtccatccctctgcacgaccacccgggcatgcacggcatgctcaaggtgctctacggcaccgtgcgcatcagctgcatggacaagctggaggcaggcggcgggcagcggcagcgagccccaccgccagaacagcagttcgagccgccgctgcagccccgggagcgggacgctgtccggcctggcgtgctccgctcgcgggccgagtacaccgaggccagcggtccctgcgtccttacgccgcaccgggacaacctgcaccaaatcgacgctgtggacgggcccgctgccttcctggacatcctggccccgccctacgaccccgacgacggccgggactgccactattaccgggtgctggaaccggtcagggccaaggaggcctccggatcgacctgtgacctgcctcgagaggtgtggcttctcgagactccgcaggccgatgacttctggtgcgagggggagccctatccaggtcccaaggtcgtcccttga

SEQ ID NO: 24 - DNA, 798 bp, Felis catus
  source 1..798: mol_type = genomic DNA
atggcctccctgatccaacggatcgcccgccaggcgtgtctcaccttccggggcagcgggggctgccgcagcgcttccgaccacggcgcggcgccgggctccgaggcccctatgcctcagggcttcccggagaacctgagcaagctgaagaacctgctgacccaagtccgcgcggaggacctgaacatcgccccgcgcaaggccacggtgcagccgttgccgcccaacctaccaccggtcacctatatgcacatttacgaaaccgacggcttcagcctcggcgtgttcctgctcaagagcggcacgtccattccgcttcatgaccacccgggcatgcacggcatgctcaaggtgctctacggcaccgtgcgcatcagctgcatggacaagctggaggcgggcggcgggcagcggccacgggccccgccgccagagcagcagttcgagccgccgctgaaagcccgggagcgggacgccgtgcggctgggcgtgctgcgctcgagggccgagtacaccgaggccagcggcccctgcgtcctcaccccacaccaggacaacctgcaccagatcgacgctgtggacgggcccgccgccttcctggacattctggccccgccctatgacccggacgatggccgggactgccactattaccgggtgctggagcctgtcagggacaaggagctttccggctcggcttgcgacctgcccagagaggtgtggctcctggagacccctcaagccgatgacttctggtgcgagggggagccctatccaggtcccaaggtcttcccttga

SEQ ID NO: 25 - DNA, 813 bp, Heterocephalus glaber
  source 1..813: mol_type = genomic DNA
atgccccgagacaacatggcttcccttatccaacggatagcccgccaggcttgcctcaccttccggggcaccgggggcgtccacggcgcttccgaccgcggcgcgacggctggccccgaggcgccgatgccccccggcttccccgagaacctgagcaagctgaagggcctgctgacccaggtccgcgccgaggacctcaacatcgccccgcgtaaggccacgctgcagccgctgcctcccaatctgccgccggtcacctacatgcacatctacgagacgcccggctttagcctcggcgtgttcctactcaagagcggcacgtctatcccgctgcacgaccaccctggcatgcatggtatcctcaaggtgctctacggcaccgtccgcatcagctgcatggacaagctggaagcgggcggtgggcagcgtccgagggcccagtcgctcgagcagcagttcgacccgccgctgcaggcccgggagcgggaggccatgcggccaggcatcctgcgctctcgggccgagtacaccgaagccagtggcccctgtgttctcacgccgcaccgagacaacctgcaccagatcgacgccgtggacggacccgctgccttcctggacatcttagccccgccctacgacccggatgacggccgggactgccactattaccgggtgctggagcctgtcaggcccaaagaggcctccagctcggcctgtgacctgccccgagaggtgtggctgctggagacgccgcaggccgatgacttctggtgcgaaggagaaccctatccaggacccaaggtcttcccttga

SEQ ID NO: 26 - DNA, 813 bp, Elephas maximus
  source 1..813: mol_type = genomic DNA
atgccccgagacaacatggcctccctgatccaacggatcgcccgccaggcgtgtctcaccttccggggtagcgggggcggccgcagcgcttccgaccgcggcgcggcgccaggccccgaggcgccgatgccgccgggcttcctagagaacctgagcaagctgaagagcctgctgacccaggtccgcgctgaggacctgaacatcgccccgcgcaaggccacgctgcagccactgccgcccagcctgccgcccgtcacctacatgcacatctacgagacggacggcttcagcctcggggtgtttctgctcaaaagcggcacgtccatcccactgcacgaccacccgggcatgcacggcatgctcaaggtgctctacggcaccgtgcgcatcagctgcatggacaagctggaggcgggcgacgggcaacggccgcgggcccctccgtccgagcagcagttcgagccgccgctgcaggcccgggagcgggacgccgtgcagctgggcgtgctgcgctcgcgggccgagtacaccgaggccagcggcctctgcatcctcacgccgcaccgggacaacctgcaccagatcgacgcagtggacgggccagccgccttcctagacatcctggccccgccctacgaccccgacgacggccgggactgccactattaccgggtgctggagcccgccgccaggcccaaggaggcctccggctcgacctgtgacctgccccgagaggtgcgtcagtcttggcatctcaggaaggactctgtcgtcactcatctgttgcagatcattctgaatcttgtactctag

SEQ ID NO: 27 - DNA, 903 bp, Mus caroli
  source 1..903: mol_type = genomic DNA
atggcctccctgatccagcgcatcgctcgccaggcgtgtctcaccttccgcggcggctcgacgggctccgaagggccggcgccgggcttcccggagaacctgagcctgctcaagagcctgctgacccaggtgcgcgccgaggacctcaacatcgcgccgcgcaaggcgctgccgcagccgctgccccgcaacctcccgccggtcacctacatgcacatctacgagacggagggcttcagcctgggcgtgttcctgctcaagagcggcacgtgcatcccgctgcacgaccacccgggcatgcacggtatgctcaaggtgctgtacggcacggtccgcatcagctgcatggacaagctggacacgggggccgggcatcggcggccgccgccagagcagcagttcgagcccccgctgcagcccttggagcgggaggccgtgcgaccgggcgtgctgcgttcccgggccgagtacaccgaggccagtgggccctgcgtgctcactccacaccgggacaacctgcaccaaattgatgctgtggacgggccagctgccttcctggacatcctggccccaccctacgacccggacgacggccgggactgccactattaccgtgtggtggagcccatcagacccaaggaggcttccggctctgcctgcgaccttccccgagaagtgtggctcctggagacaccacaggccaacaacttctggtgtgagggagagccctatccccaaggtcctaccttgaagctcctgccgcccggggaactgtgggccaaagatgtaccctgccctgctcaagaaaggcctggctgccctctaccttccataagggctcttctctctctgccccctcacctggaccatggatttactggagtgaccagcgccacatcttga

SEQ ID NO: 28 - protein, 270 aa, Capra hircus
MPRDNMASLIQRIARQACLTFRGSGGGRSSSDFGEAPGPEAPIPQGFPENLSKLKSLLTQVRAEDLNISPRKATLQPLRPNLPPVTYMHICETDGFSLGVFLLKSGTSIPLHDHPGMHGILKVLYGTVRISCMDKVEVGSGQRPRAPPPEQQFEPPLQPRERDAVQPGVLRSRAEYTEASGPCFLTPHRDNLHQIDAVDGPAAFLDILAPPYDPDDGRDCHYYRVLEPVRAKEASGSACDLPREVWLLETPQADDFWCEGEPYPGPKVFP

SEQ ID NO: 29 - protein, 270 aa, Homo sapiens
MPRDNMASLIQRIARQACLTFRGSWGGRGASDRDAASGAEAPMQPGFPENLSKLKSLLTQLRAEDLNIAPRKATLQPLPPNLPPVTYMHIYETDGFSLGVFLLKSGTSIPLHDHPGMHGMLKVLYGTVRISCMDKLDAGGGQRPRALPPEQQFEPPLQPREREAVRPGVLRSRAEYTEASGPCILTPHRDNLHQIDAVEGPAAFLDILAPPYDPDDGRDCHYYRVLEPVRPKEASSSACDLPREVWLLETPQADDFWCEGEPYPGPKVFP

SEQ ID NO: 30 - protein, 273 aa, Ursus maritimus
MPRDNMASLIQRIARQACLTFRGSGGCRSASDLGAAPGPEAPMPQGFPENLSKLKNLLTQVRAEDLNIAPRKATLQPLSPNLPPVTYMHIYETDGFSLGVFLLKSGTSIPLHDHPGMHGMLKVLYGTVRISCMDKLEAGGGPQPRAPPPEQQFEPPLQARERDAVRPGVLRSRAEYTEASGPCVLTPHQDNLHQIDAVDGPAAFLDILAPPYDPDDGRDGRDCHYYRVLEPVRDKEAPGSACDLPREVWLLETPQADDFWCEGEPYPGPRVFP

SEQ ID NO: 31 - protein, 270 aa, Nycticebus coucang
MPRDNMASLIQLIARQACLTFRGGGGGRRASDRGAASGPEAPMPPGFPENLSKLKSLLTQIRAEDLNIARRKATLQPLPPNLPPVTYMHIYETEGFSLGVFLLKSGMSIPLHDHPGMHGMLKVLYGTVRISCMDKLEAGGEQRPRAPPLEQQFEPPLQPRERDAMRPGVLRSRAEYTEASGPCVLTPHRDNLHQIDAVDGPAAFLDILAPPYDPDDGRDCHYYRVLEPVRSKEASGLACDLPREVWLLETPQADDFWCEGEPYPGPKVFP

SEQ ID NO: 32 - protein, 256 aa, Mus musculus
MPRDNMASLIQRIARQACLTFRGSSTGSEGPAPGFPENLSLLKSLLTQVRAEDLNIAPRKALPQPLPRNLPPVTYMHIYETEGFSLGVFLLKSGTCIPLHDHPGMHGMLKVLYGTVRISCMDKLDTGAGHRRPPPEQQFEPPLQPLEREAVRPGVLRSRAEYTEASGPCVLTPHRDNLHQIDAVDGPAAFLDILAPPYDPEDGRDCHYYRVVEPIRPKEASGSACDLPREVWLLETPQADDFWCEGEPYPGPKVLP

SEQ ID NO: 33 - protein, 250 aa, Salvelinus alpinus
MMPRDNMTSLVQKIGRQALVTFRNPSLVGDKAFLDNQGKLQTLMTEIRAADLKIAPRKVDCASTPLPHNPPVTYMHICETDQFSMGVFLLKSGASIPLHDHPGMYGMLKVMYGKVRITCFDRLDKSTNVASDTQFKPPLLPFQRDALRMSILRSVGEFTEESGPCILTPDRDNLHQIDAVDGPTAFLDILAPPYDPNDGRDCHYYKVLESDSEDKKAEAQGQKEVWLMEVPQPSDFWCGGEPYPGPEVNI

SEQ ID NO: 34 - protein, 278 aa, Phrynosoma platyrhinos
MPRDNMAASLIQRVARQARLTFRPPGTTTTSGGGGGGDGPRSAEALHRLRDLLGEVRAEDLRLSPRGPSAVPGRAQPPVSYMHICETESFSMGVFVLRAGACIPLHDHPGMNGLLKVLYGTLRIACLDTLPEDHDDDDDGGGGGGGAGASGPPLPHGRHHRHRHRGRHRALLRSHRLYTPSSAPCLLSPETDNLHQIDAVGGGPAAFLDILAPPYDPEHGRDCHYYRLLDGPGLLPPPPPSPEEDPPGLPKELWLQETPQAPDFWCGGEPYPGPQVSP

SEQ ID NO: 35 - protein, 284 aa, Vombatus ursinus
MPRDNMATLIQRIARQARLTFRRGGGGGGAPEAGFPENLQQLQQLLSQLRAEDLNISPRRAALPSPPPARPPHLPPVTYMHICETAEFSLGVFLLKSGTSIPLHDHPGMHGVLKVLYGTLRISCLDPLPGEGPGAPREGPQPQPHPPPPQQAPAAPPRFEPPLRPGQRAVVRRALLRSRAEYTEASAPCLLSPHRDNLHQIDAVDGPAAFLDILAPPYDPDDGRDCHYYRLLEAAAPAGPPDAGLGCGPAAPLPVPREVWLLETPQAADFWCEGEPYPGPKVTP

SEQ ID NO: 36 - protein, 247 aa, Notechis scutatus
MPRDEMAASLIQRVARQARATFGAGAGEAPAELQRLRELLGQVSAEDLGLLQPRGPSAVPGPSQPPVSYMHICETARFSMGVFVLRAGACIPLHDHPGMQGLLKVLYGTLRIACFDAADEEPEEQPDAASGAASSSPRPRRLRALLRSHRLYTAAAPPCLLSPQRDNLHQIAAEEAAGPAAFLDILAPPYDPARGRDCHYYRLADGQSLPPPPAQPVPPSALWLLETPQAPDFWCGGEPYPGPRVWP

SEQ ID NO: 37 - protein, 251 aa, Salmo salar
MPRDNKKSLIQKIANQANITFKDFNASAIGDNKVLLDNKGDLITLLTEIRAADLKMKPNFSHSEHNPPVTYMHICETESFSMGVFLLNNGASIPLHDHPGMNGMLKVLYGKVSISCFDLLDKPSDQVQSEIQFDPPLLPFQKDSLRRAVLRSTMWFTHDSSPCILTPQRENLHQIDTVDGPAAFLDILAPPYDPEDGRDCHYYKVLQTVPDADSKVEAQQQWEEETWLLEIGQPDGFWCGGEPYPGPKVSF

SEQ ID NO: 38 - protein, 242 aa, Adelges cooleyi
MASKIECVLKQSLITFSGSHTSKTFHDNLIKLKRYANELDANSITLDNRLLRCDTYELNSSFDKLSGSVLIRRVRAPVTYIEVYEDQNVSIGIFVLRNGAKIPLHDHPCMYGVLKVIHGKVRIQSYTPKKKDYSQLNECIISAVKMAPIIIGESDEPCVLCPVEGNIHQVDTVDGPAAFVDILAPPYRTDIPDVGKRYCRYFKELNMSKDIKLEVVPDPKDYWSDTAPYTGPQFNLNIDENS

SEQ ID NO: 39 - protein, 194 aa, Lipotes vexillifer
MPRDNMASLIQRIARQACLTFRGGSLLKSGTSIPLHDHPGMHGILKVLYGTVRISCMDKLEVGSGQRPRAPPPEQQFEPPLQPRERDALRPGVLRSRAEYTAASGPCVLTPHRDNLHQIDAVEGPAAFLDILAPPYDPDDGRDCHYYRVLEPVRAREASGSACDLPREVWLLETPQADDFWCEGEPYPGPKVFP

SEQ ID NO: 40 - protein, 245 aa, Mytilus edulis
MVSNIQKVAQTAYRLFSRICGNSDVLLSSQNVGLLTSMMNTIKDKDVNFDMSKISLESETPVSYIDIYEDPVMTMAIFIIRDGCKIPLHDHPGMYGFCKVIHGSARMKSFSDPQNFVGNPPENIASKIRPYLRQSIKPVVLEEESIFSSDDNCCVVTPNHGTYHELVAVNGPVAFLDILAPSYDHASGARICQYYEEIKGQNEEIKHLGKPLIWFTPISQPSDFWCDPEEYEGPSVVDALNEIVK

SEQ ID NO: 41 - protein, 228 aa, Pan troglodytes
MQPGFPENLSKLKSLLTQLRAEDLNIAPRKATLQPLPPNLPPVTYMHIYETDGFSLGVFLLKSGTSIPLHDHPGMHGMLKVLYGTVRISCMDKLDAGGGQRPRALPPEQQFEPPLQPREREAVRPGVLRSRAEYTEASGPCILTPHRDNLHQIDAVEGPAAFLDILAPPYDPDDGRDCHYYRVLEPVRPKEASSSACDLPREVWLLETPQADDFWCEGEPYPGPKVFP

SEQ ID NO: 42 - protein, 254 aa, Manis javanica
MPRDNMASLIQRIARQACLTFRGSGGGLSASDRCTAPGPEAPMPQGFPENLNKLKSLLTQVRAEDLNIAPRKATLQPLPPNLPPVTYMHICETDGFSLGVFLLKGGTCIPLHDHPGMHGMLKVLYGTVRISCIDKLEAGGGQRPRAPPPEKQFDPPLQARERDAVRPGVLRSRAEYTEASGPCVLTPHRDNLHQIDAVDGPAAFLDILAPPYDPDNGRDCHYYRVLEPIRAKEASGSACDLPREGSAHYYSTLT

SEQ ID NO: 43 - DNA, 1857 bp, Propithecus coquereli
  source 1..1857: mol_type = genomic DNA
atggtaaaatgctttcccttgaggaaaacagaacagcactctgaagtttgtccacacggagcctcctccggcaattcaccaacttcaatccaagcaatccttaagatacggtactggcagcacaggactcagccccagggaactttggacagagacacacacacgggacaacgctatctgaagagacacagggagaagacagccatttgcaagtcaaagagagaagcctggaacgtagccttccctcgccgccacaggaggaaacgaccctgctggggactcctaacctccagaatttcactggacttcagcatgatcacttctccgtcgctggcttatgtggcgattttggttttctgtgtctcaaaaaccagctctctggacacttttattgcagctgtttatgagcatgcggtgctattgcctaataccagcctcacaccagtgtctcgtgaggaggcgctggcgttaatgaatcggaatctggatcttttggaaggagcgatcgcgtcagcagcggagcagggcgcacatattatagtgaccccagaagatggtatttatggctggaacttcaacagggaatctctctacccgtacttggaggatatcccacaccctcaagtaaactggatcccctgtaatgatcctaacagatttggccacacccccgtacaagaaagactcagctgcctggccaaggagaactctatctatgttgtggcaaatattggggacaagaagccatgtaatgccagtgaccctcagtgtccccctgatggccgttaccagtacaacactgatgttgtgtttgattctcagggaaggctggtggcacgctaccataagcaaaatcttttcttgggtgaagatcaattcaatgcacccaaggagcctgaggttgtgactttcaacaccacctttggaaggtttggcatttttacgtgctttgatatactcttccaccatcctgctgttaccctggtgaaagatttccacgtggacaccatacttttcccaacagcttggatgaatgttttgccacatttgtcagctattgaattccactcagcgtgggctatgggcatgggggtcaatttccttgcatctaatatacattacccctcaatgaggatgacaggaagtggcatttatgcacccaattctgcaagagtatttcattatgatatgaagacaaaggagggaaaactcctcctctcacaactggattcccacccacaccaccctgtagtggtgaattggccatcttatgccagcagtgtggaagcactctcgactggaaaccaggaatttaagggcattatcttttttgatgaatacaccttcgtggagctcaaaggagtcggggggaattacacagtatgtcagaaagatctctgttgtcatctaagctacaagatgtcagagaagagatcagatgaagtttatgccctaggggcatttgatggactgcacaccgtggaaggacgctattatctacagatttgtactctggtgaagtgtaaaacgaccgacttaaacacttgtggtgactcagttgaaaccgcttctaccaggtttgaaatgttctccctcagtggcacttttggaacccagtatgtcttccctgaggtgttgttgagtgagattcagcttgcacctggagaatttcaggtgtcaagtgatggacgcttgtatagcctgaagccaacatctggacctatcttaacagtgactctgtttgggaggttgtatgagaaggacggagcatcaaacgcttcatcagacctcatagcgcaagcactaagaataatgctagcagttataatacctattgtatactcattaagttggtag

SEQ ID NO: 44 - DNA, 1545 bp, Equus asinus
  source 1..1545: mol_type = genomic DNA
atgatttcttctcagctgctcgcttatgtggcagttttggttttctgcaccttccaagcccgctccctggagacctttattgcagctgtttacgagcacgcagtgatattgcccaatgccaccctaacgccagtgtcccgggaagaggcattgctattaatgaaccgcaatctggatcttctggagggagcaatcgcctcagcagccaagcagggtgcgcacatcatcgtgactccagaagatggtgtttacggctggaacttcagcagggacacgctctatccgtacttggaggacatcccagaccccgaagtgaactggatcccttgcaacaatccccacagatttggccacactccagtccaagagaggctcagctgcctggccaaggacaactctatctacgtcgtggcaaatttcggggacaagaagccatgcaatgccagtgaccctgagtgcccccctgatggccgttaccagtacaacactgatgtggtgtttgatgctcagggaaaacttgtggcacgctaccataagcaaaaccttttcatgggtgaagatcaattcaatgcacccaaggagcctgaggtcgtgactttcaacaccacctttggaaagtttggcattttcacgtgcttcgatattctcttctacgaccctgctgttaccctggtgacagatttccaagtggacaccatcctcttcccgacagcctggatgaatgttttgccacatttgtcagctattgaatttcactcggcttgggctatgggcatgggggtcaatttccttgcatccaatatacattacccctcaatgaaaatgacaggaagtggcatctatgctcctgattctccaagagcatttcattatgatatgaagacggaggagggaaaactactcctctcacagctggactcccacccgcaccacccagcagtggtgaactggacttcttttgccagtggtgtgaaaacactctcaacgggaaaccaggaatttaacagcactgtcttttttgatgaattcaccttcctggagctcaccgaagtcacaggaaattacacagtttgtcagaaagctctctgctgtcacctaagctacaagatgtctgagaagagatcagatgaagtgtacgccctaggggcatttgatggcttacatactgttgaagggagttattatttacaggtttgcactctgttgaagtgtagaaccacggacttacacacttgtggtgactcggtggaaactgcttccactaggttcgatacgttctccctcagtggcacttttgggacccagcatgtcttccctgaggtgctgctgagtgaaattcagctcgcacctggagaattccaggtgtcaagtgatggacgcttgttcagtctgcagcccccctcgggacctgtcctgacagtgactctttatgggaggctgtacgagaaggaccaggtatccgacgcctcatcagacctcacagcacaagcactgagagtaatgctcgtagttctaatacctgttgggttctcaataagttggtag

SEQ ID NO: 45 - DNA, 1383 bp, Neomonachus schauinslandi
  source 1..1383: mol_type = genomic DNA
atgaaccgaaatctggatcttttggaggcagcaatcacttcagcagcaaaccagggtgcacatattattgtgactccagaagatggtatttatggctggaactttaccagggaaactatctacccatacctggaggatatcccagaccctggagtgaactggatcccttgtaataatcccaaaaggtttggctacaccccagtgcaagaaagactcagctgcctggccaaggacaactctatctacgttgtggcaaatattggggacaagaagccatgcaatgccagtgaccctcagtgtccccccgatggtcgttaccagtacaacaccgatgtggtgtttgattctcagggaaaactggtggcacgctatcataagcacaaccttttcatgggtgaaaatcaattcaatgtacccaagaagcctgaggttgtgacttttgacaccatctttggaagatttggcattttcacgtgcttcgatatactcttctatgatcctgctgttactctggtgaaagatttccatgtcgataccatagtcttcccaacagcatggatgaatgttttgccacatttgtcagctattcagttccactcagcttgggcaatgggcatgggggtcaacttccttgcatccaatatacatcacccctcaaagagaatgacaggaagtggcatctatgcacctgattctccaagagcatttcattatgatatgaagacaaaggagggaaaactccttctctcacaactggattctcactcacacagccctgtagtgaattgggcttcttatgccagtagtataaaggcattctcaacaggaaatcaggaatttaagggtactgccttttttgatgaattcaccttcttggagctcacaagagtcacaggaaattacacagtttgtcagaaaaaactctgctgtcatctaagctacaagatgtctgaaaagagaagagatgaagtttacgccctgggggcatttgatggactacatattgtcgaagggacgtattatttacagatttgtaccctgctgaaatgtaaaacaacaaacttaaacatttgtggtggtgcagttgaaacagcttctaccaggtttgaaatgttttccctcagtggcacttttggaactcagtatgtcttccctgaggtgttgctaagtgaaagtcagcttgcacctggagaatttcaggtgtcgagtgatggacgcttgttcagcgtgaagccgccatctggacctctcttgaccgtgactctatttgggagaatgtatgagaaggaccagacattaaaggcttcatcagacccccaggcacaaacactgggaataatgctcttagtcataatgccagttgtgtgctcattaagttggtag

SEQ ID NO: 46 - DNA, 1545 bp, Carlito syrichta
  source 1..1545: mol_type = genomic DNA
atggttgcttctcagttgctggcttatatggcaatcttggtgttctgtgtcttaaaagccagctccctggacacgtttcttgcagctgtttatgagcatgcagtgatactgcctgatgccaccctgacaccagtgtctcgtgaagaagcattggtgctaatgaataggaatttggatcttctggaaagagcggtcatatcagctgcacagaagggtgcacatattattgtgactccagaagatgccatttacggctggaacttcagcagggaatctgtctacccctacttggaggatatcccacaccctcaagtgaactggatcccttgtaataatccgaacagatttggccacactccagtccaagaaagactcagctgcctggccaagaacaactccatctatgtcgtggcaaatattggagacaagaagccatgcaatgccagtgaccctcagtgtccccctgatggccgttaccaatacaacactaatgtggtatttgattctcagggtacgctggtggcacgctaccataagcagcaccttttcataggtgaagatcacttcaatgcacccaagagccctgaggtcgtgactttcaacactacatttggaaggtttggcattttcacatgctttgatatactcttccatgatcctgcagttaccctggtgaaagatctccacgtgaacaccatactatttcccacagcttggatgaatgttctgccacatttgtcagcgattgaattccactcagcttgggctatgggcatgggagtcaacttccttgcctccaatatccattaccctttgatgaaaatgacaggaagtggcatctatgcacccaattctccaagggcatttcattatgatatgaagacagaagaaggaaaactcctcttctcgcaactggatgcccgcccataccaccccagagtggtgaattggacttcttacgccagtagtatagaggcagtctcatcagaaaaacagaaatttaagggagtcgtcttctttgatgaattcaccttcgtggagctcacagatgttatgggaaattacacagtttgtcagaaagatctctgctgtcatctgagctacaagatgtctgagaagagatcagatgaagtttacgcccttggggcatttgatgggctgcacacggtggaagggtgctattatctacaggtttgtactctgctgaagtgtaaaacaactgacttacacacgtgtggtgactcagtggaaacggcttctaccacgtttgagatgttctccctcagcggcacctttggaacccaatatgtcttccctgaggtgctgctgagtgaaatgcgacttgcacccggagaatttcaggtgtcagatgatggatgcttgtctagcctgaagccaacatctagacctgtcttaacagtgactctttttgggaggttgtatgagaaggattgtgcatcaaatgcttcatcagacctcacagcacaagcacgaagaaccacgctgatagtaatgatatgtattgtccatttattaagttggtag

SEQ ID NO: 47 - DNA, 1632 bp, Dipodomys ordii
  source 1..1632: mol_type = genomic DNA
atggtgattgctcggttgctggcttatttggcattttcagttttctgtgtcttaccagccaactgcctggacacttttactgcagctgtttatgaacatgccgtaatactgcctaacttcaccacaacaccagtgtctcgtgaagaggcactggtgttaatgaatcagaatctagacattttggaagaagcaatcacctcagcagcaaaacagggtgcacaaattattgtgactccagaagatggtgtttatggtgtgaatttcagcagggacaccatttacccatatttagaggacattccagacccggaagtcaattggatcccctgtaatcatcccaaaaggtttggtgatactcaaatacaagaaagactcagctgtctggccaagagcaactctatctatgtggtggcaaatataggagacaagaaaccatgcaatgccagtgaccctcgatgtccccctgatggtcgttaccaatacaacactaatgtggtgttcgattcacagggaaaactggtagcccgctaccacaagcaacatcttttcatgagtgaatcttttcacttcaatgcacccaaagatcctgaggtcgtgactttcaacactccctttggaaaatttggcatttttacatgctttgacatcctcttctacgaccctgccattaccctggtgaaagatttccatgtggacactatcctcttcccaacagcttggatggatgttctaccacacttgtcagccattgaattccactcagcttgggctatgggcatgggagtcaatttacttgcatccaatttacatcgcccccagaacagaatgacaggaagtggcatctatgcacctgattctccaagagcttttcattatgacaggaagacagaaaagggaaagctcgtcctctctcagctggattcccacccctaccgccccgaagtgaactggagttcttatgccagcaacataggagcactctcaactggaaaccaagaatttaagggcagtgccttttatgatgagtacactttcgtggagctcacaggaatcacaggaaattacactgtttgccagaaagatctctgctgtcatctaaactaccagatgtctgagaagagatccaatgaaatttatgctctaggggcatttgatggactgcacacagttgaagggtgttaccatctacaggtctgcactatgttaaagtgtaaaaccactaacagacatacctgtggtgcttcagttgacacggcttctactaggtttgaaatgttctccctcagtggcacttttgggacccagtatgtctttcctgaggtgttgttgagtgaagttcggctcgcacctggagaatttaaggtaacaacctttgcttcatttccccagttaattgcatgtgaaaggaaatactggtttatttcaaaagctactgaaatcatacatatttccattgggaaaagggaaagattatctttgaggatcaggaaaggcaccgcaagcagcctctgccataaagtcttaccaccagcatgttctgccttactatggacttaccaacacacccaaggatcactggccataggaatgaaaaatctgagtgacacagagaaagcagctttatttatatga

SEQ ID NO: 48 - DNA, 1257 bp, Ictidomys tridecemlineatus
  source 1..1257: mol_type = genomic DNA
atgttttggaaggagcgatcacgttagctgcaaatcagatttggctacaacccagtacaagaaagactcagctgcctggccaagaataactctatctatgtggtggcaaatataggggacaagaagtcatgcaaagccagtgagcctcagtgtcctcctgatggccgttaccaatacaatactaacgtggtgtttgattctcagggaaaactagtggcccgctaccataagcaaaaccttttcatgggggaagaccaattcaatgcacctaaagagcctgagatggtgacatttaacacaacctttgggaagtttggcattttcacttgctttgatattctcttccatgatcctgctgttgccctggtgaaagacctccacgtggacaccgtactcttcccaacagcttggatgaatgtcctgccacatttgtcagcggttgagttccactcagcttgggctatgggcatgggagtcaatttccttgcatctaatttacataacccctcaaatagaatgacaggaagtggtatttatgcacctgattttccaagagcatttcattatgatatgaagacaccagagggaaaacttatcatctcaacactggattctcacccatcccattccatagtgaattggtcttcttacgccagcagtatacatgcattcccaacaggaaaccaagaattcaagggcactgtcttttttgatgaattcacctttgtggagctcacagaaattgcaggaaattacacagtttgtcagaaagagctctgctgtcatttaagctatgaaatgtctgaaaagagatcagatgaagtttatgccttaggagcatttgatggactgcacgttgttgaagggcgctattacctacagatttgtactctgctgaaatgtgaaaccactgacttgcacacctgtggtggttcagttgacacagcttccactaggtttgaatcgttctccctcagtggcactttcggaacccaatatgtcttccctgaagtactgctgagtgacgttcagcttgcacctaaagaatttcaggtgtcaagtgatggacgtttgtttagcttgaagccaacatctggacctctgttaacggtcactctctttgggagggtgtacgagaaggatatggcatcaaatgggtcacctgacctctcaacatattcagtaaagataatgttagtagttatgatacctattgtgttctcgttatgttggtagaatatttacattttctctctt

SEQ ID NO: 49 - DNA, 1539 bp, Rattus norvegicus
  source 1..1539: mol_type = genomic DNA
atgagcctgtctcggtggctggcgtgggctgcagcgttttccgtcttctgtgtcttaaaagtcagctctctggacactttcctcgcagctgtttacgagcatgctgtgatcctgcctaaagtcaccctgttacctgtgtctcacagtgaggcactggcattaatgaaccagaatctggaccttctggaaggagcgatcttatcagcagctaagcagggtgcgcacattattgtgactccagaagatggtatatacggtgtgcagttcaccagggataccatctacccatacctggaggatatcccagaccctcaagtaaactggataccctgtgataatccggaaagatttggctctaccccggtgcaggagagactcagctgcttggccaagaacaactcaatctatgtcgtggcgaacatgggagacaagaagccatgcaacaccagcgactctcactgtccacctgacggccgattccaatacaacactgatgtggtgtttgattcccggggtaaattggttgccagataccataagcaaaaccttttcatgggagaagagcaattcaatgcccccccggagcctgaggtcgtgacttttgacaccccctttggaaagtttggcatcttcacctgtttcgatattctcttccatgatcctgctgtcaccctagtgacagagttccaggtggacaccatactgttcccaactgcctggatggacgtgcttcctcatttggcagccattgaattccactcagcttgggctataggcatgggagtcaatttcctagcagctaatctacatattcccttgaggagaatgacaggaagtggtatctatgcacccgattctccacgggcctttcattatgacaggaagacacaggaaggaaagctcctccttgcacagctggactctcacccgagtcactccccagtgaactggacttcctatgccagcagtgtagaagcacccccaactgaaaaggaggaatttcggagttttgttttctttgatgagtttacctttgtggagctcaaagggatcacaggaaattacactgtttgccagaacgacctctgctgtcatctaagctaccagatgtctgagaagcgagctgatgaagtttatgctcttggggcttttgacgggttgcacaccgtggaagggcagtatcacctgcagatttgcattctgctaaagtgtaaaactaccaatttacgcacctgtggtagttcagtggacacagcttctaccaggtttgaaatgttctccctcagtggcacttttggaacgcggtatgtcttccctgaggtgttgctgagtgaagtcaagcttgctcctggggagtttcaggtgttgagtgacgggcgcctggttagcctgaagccaacgtctggacctgtgttaacagtcggtctctttgggaggttgtatgagaaggactgggcatcaaatgcttcctcagacctcatagcacactcactgataataatgctgatcctagctcctattatacattacttgtgttaa

SEQ ID NO: 50 - DNA, 1542 bp, Homo sapiens
  source 1..1542: mol_type = genomic DNA
atgactactcagttgccagcttacgtggcaattttgcttttctatgtctcaagagccagctgccaggacactttcactgcagctgtttatgagcatgcagcgatattgcccaatgccaccctaacaccagtgtctcgtgaggaggctttggcattaatgaatcggaatctggacattttggaaggagcgatcacatcagcagcagatcagggtgcgcatattattgtgactccagaagatgctatttatggctggaacttcaacagggactctctctacccatatttggaggacatcccagaccctgaagtaaactggatcccctgtaataatcgtaacagatttggccagaccccagtacaagaaagactcagctgcctggccaagaacagctctatctatgttgtggcaaatattggggacaagaagccatgcgataccagtgatcctcagtgtccccctgatggccgttaccaatacaacactgatgtggtatttgattctcaaggaaaactggtggcacgctaccataagcaaaaccttttcatgggtgaaaatcaattcaatgtacccaaggagcctgagattgtgactttcaataccacctttggaagttttggcattttcacatgctttgatatactcttccatgatcctgctgttaccttggtgaaagatttccacgtggacaccatagtattcccaacagcttggatgaatgttttgccacatttgtcagctgttgaattccactcagcttgggctatgggcatgagggtcaatttccttgcatccaacatacattacccctcaaagaaaatgacaggaagtggcatctatgcacccaattcttcaagagcatttcattatgatatgaagacagaagagggaaaactcctcctctcgcaactggattcccacccatcccattctgcagtggtgaactggacttcctatgccagcagtatagaagcgctctcatcaggaaacaaggaatttaaaggcactgtctttttcgatgaattcacttttgtgaagctcacaggagttgcaggaaattatacagtttgtcagaaagatctctgctgtcatttaagctacaaaatgtctgagaacataccaaatgaagtgtacgctctaggggcatttgacggactgcacactgtggaagggcgctattatctacagatttgtaccctgttgaaatgtaaaacgactaatttaaacacttgcggtgactcagctgaaacagcttctaccaggtttgaaatgttctccctcagtggcactttcggaacccagtatgtctttcctgaggtgttgctgagtgaaaatcagcttgcacctggagaatttcaggtgtcaactgacggacgcttgtttagtctgaagccaacatccggacctgtcttaacagtaactctgtttgggaggttgtatgagaaggactgggcatcaaatgcttcatcaggcctcacagcacaagcaagaataataatgctaatagttatagcacctattgtatgctcattaagttggtag

SEQ ID NO: 51 - DNA, 1539 bp, Mus musculus
  source 1..1539: mol_type = genomic DNA
atgggcatgtcttggtggctggcgtgtgctgcagcgttttctgccctctgtgtcttaaaagccagttcgctggatactttcctcgcggctgtttacgagcatgctgtgatcctgcctaaggacaccctgttgccagtgtctcacagtgaggctctggcattaatgaaccagaatctggaccttctggaaggagcgatcgtatctgcagcgaacgagggtgcgcacattattgtgactccagaagatggcatatacggtgtgcgtttcaccagggatacgatctacccatacctggaggagatcccagaccctcaagtaaactggataccctgtgataaccctaaaagatttggctctaccccggtgcaggagagactcagctgcttggccaagaacaactccatctatgttgtggcgaacatgggagacaagaagccgtgtaacaccagcgactctcactgtccacctgacggcagattccagtacaacactgatgtggtgtttgattcccagggtaaactggttgcgagataccataagcaaaacattttcatgggagaagatcagttcaatgtccccatggagcctgagtttgtgactttcgacaccccctttggaaagtttggcgtcttcacctgtttcgatattctcttccatgatcccgctgtcaccctggtgacagaattccaggtggacaccatactgttcccaaccgcctggatggacgtccttcctcatttggcagccattgaattccactcagcttgggctatgggcatgggggtcaatttcctagcagctaatctacataatccctcgaggagaatgacaggaagtggtatctatgcacccgattctccaagggtctttcactacgacaggaagacccaagaaggaaaactcctcttcgctcagctgaaatcccacccaattcactccccggtgaactggacttcctatgctagcagtgtagaatcaaccccaaccaaaacccaggaatttcagagtattgtcttttttgatgagtttacctttgtggagctcaaagggatcaaaggaaattacactgtttgccagaatgacctctgctgtcacctaagctaccagatgtctgagaagcgagcagatgaggtttatgcctttggagcctttgatgggctgcacaccgtggaagggcagtactacctacagatctgcatcctgctaaaatgtaaaactaccaatttacgcacctgtggtagttcagtggacacggcttttaccaggtttgaaatgttctcgctcagcggcacttttggaacccggtatgtcttccctgaagtgttgctgagtgaggtcaagctcgcacctggggagtttcaggtgtcaagtgatgggcgcctggttagcctgaagccaacctcgggacctgtgttaaccatcgggctctttgggaggttgtatgggaaggactgggcatccaatgcttcctcagacttcatagcacactcgctgataataatgctgattgtgacgcctattatacattacttgtgctga

SEQ ID NO: 52 - DNA, 1311 bp, Mesocricetus auratus
  source 1..1311: mol_type = genomic DNA
atgctgtgatcctgccaaatgacaccctgacacccgtgtctcacagtgaggcgctggcactaatgaatcagaatctgaatcttctggaaggagcgatcgtgtctgcagcaaagctgatttggctacaacccagtacaggagagactcagctgcttggccaagaataattcaatctatgttgtggcgaacatgggagacaagaaaccatgcaacactagtgaccctcagtgtcctcctgatgggcgtttccagtacaacactaatgtggtgtttgattccctgggcaaactggttgcacgataccataagcaaaaccttttcatgggtgaagaacaattcaacgcacccatggagcccgagttggtgactttcaacaccccgtttggaaggtttggcatcttcacatgcttcgatatcctcttccacgatcctgctgtcaccctggtgaccaagtttcaggtggacaccatactgttcccaaccgcttggatggacgttctgcctcatttggcagccattgaattccactcagcttgggctatgggcatgggggtcaatttacttgcagccaatatacatagtccctcgaggagaatgacaggaagtggtatctatgccccagactctccaaaggcctttcactatgacaggaaaaccaatgagggaaaacttgttctagcgcagctggattcccacccaagtcgctccactgtgaactggacttcttatgccagcagtgtagaagcacccgcaacaggaaaccaggaatttcagagcatcattttctttgatgagtttacctttgtggagctcaaaggaatcacaggaaattacactgtttgccagaatgatctctgctgtcatctaagctaccagatgtctgagaagcgagctgatgaagtttatgcccttggggcatttgatggcttgcacactgtggaagggcagtatcatctgcagatctgtactctgctgaaatgtaaaaccaccagtttacacacctgtggtgattcagtggacacagcttctaccaggtttgaaatgttctccctcagtggcacatttggaactcagtatgtcttccccgaggtgttgctgagtgaagtcaagcttgcacctggggaatttcaggtgtcaagtgacggacgtttgtttagcctgaagccaacatctggacctgtgttaacagtcactctgtttgggaggttgtatgagaaggactcggcatctagtgcttcctcagacctcataacacactcactaatgataataacggtgattgtcctacctattgtacattacttatgttaa

SEQ ID NO: 53 - protein, 512 aa, Physeter catodon
MSQLLDYVAIFFFCVLKASSLDTFTAAVYEHAVILPNATLIPVSHEEALLLMNQNLDLLEAAITSASKQGAHIIVTPEDGIYGFNFNRESIYPYLEDIPDPQVNWIPCNNPDRFGHTPVQERLSCLAKDNSIYIVANIGDKKPCNTSDPHCPPDGRYQYNTDVVFDSEGKLVARYHKQNLFLGEDQFNAPKKPEVVTFNTTFGKFGIFTCFDILFHDPAMTLVKDFHVDTILFPTAWMNVLPHLSAIEFHSAWAMGMGVNFLASNLHYPSMKMTGSGIYAPDSPRAFHYDMKTEKGKLLLSQLASHPHPTAVVNWTSYASGIEAPSMGNQEFKGIIFFDEFTFLELKEVTGNYTVCQKDLCCHLSYKMSEKRSDEVYVLGAFDGLHTVEGSYYLQICTLLKCKTMDFHTCGDSVETASTRFETFSLSGTFETQYVFPEVLLSEIQLAPGEFQVSSDGRLFSQKPLSGPILTVTLFGRLYKKDSAPNAFSDLMAQALRVMLIVVIPIVYSLGW

SEQ ID NO: 54 - protein, 514 aa, Myotis myotis
MITSQLLTCVAISIFCALKASSLDTFIAAVYEHAVILPNATLAPVSRELALGLMNRNLDLLEGAITSAAKQGAHIIVTPEDGLYGWNFNRESIYPYLEDIPDPQVNWIPCNHPNRFGHTPVQERLSCLAKENSIYVVANIGDKKPCNASDPQCPSDGRYQYNTDVVFDSQGKLVARYHKQNLFLGEDQFNVPKEPEVVTFDTTFGRFGIFTCFDILFHDPAVALVKDFHVDTILFPTAWMNVLPHLSAIEFHSAWAMGMKVNFLASNIHHPSKKMTGSGIYAPDSPRAFHYDMKTEKGKLLLSRLDSHPKHPVMVNWTSYASGIEAFSMRNKEFKGTVFFDEYTFLELRGVTGNYTVCQKDLCCHLSYKMSEKRADEVYALGAFDGLHTVEGNYHLQICTLLKCKTTSLHTCGHAVETASTRFEMFSLSGTFGTQYVFPEVLLSEIQLAPGEFQVLSDGRLLSLKPPPGPVLTVTLFGRVYEKDHTSNASSGFMAYTLTIMLIVITSILYSLSR

SEQ ID NO: 55 - protein, 512 aa, Mus musculus
MGTSWWLACAAAFSALCVLKASSLDTFLAAVYEHAVILPKDTLLPVSHSEALALMNQNLDLLEGAIVSAAKQGAHIIVTPEDGIYGVRFTRDTIYPYLEEIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGVFTCFDILFHDPAVTLVTEFQVDTILFPTAWMDVLPHLAAIEFHSAWAMGMGVNFLAANLHNPSRRMTGSGIYAPDSPRVFHYDRKTQEGKLLFAQLKSHPIHSPVNWTSYASSVESTPTKTQEFQSIVFFDEFTFVELKGIKGNYTVCQNDLCCHLSYQMSEKRADEVYAFGAFDGLHTVEGQYYLQICILLKCKTTNLRTCGSSVDTAFTRFEMFSLSGTFGTRYVFPEVLLSEVKLAPGEFQVSSDGRLVSLKPTSGPVLTIGLFGRLYGKDWASNASSDFIAHSLIIMLIVTPIIHYLC

SEQ ID NO: 56 - protein, 513 aa, Homo sapiens [sequence omitted]

SEQ ID NO: 57 - protein, 512 aa, Monodelphis domestica
MIPFQLQSYIAFFALWAFKTNCLDTFIAAVYEHAVILPPDNIKLDSREEALALMNENMDILEEAVISAAKQGAHIIVTPEHGIYGFIFNRVNLYPYLEDIPHPKVNWTPCTDDSRFGSTPVQKRLSCMARNNSIYMVANIGDRKSCDPTESKCPSNGYYQYNTNVVYDSEGKLVARYYKKYMLMNEAQFDTPTEPQLVTFDTTFGKFGILTGFDILFHDPVVPLVEKLKVDTILLPVAWMNTLPHLYPTAFHAAWAMGMRVNLLVANIHSISNQMTGSGIYAPDSVKAYYFDKKSDDGKLLVSELESHPIVRPPVNWTLYASSIKPFSSEQEEFKALVLFDMFTFKELINDTGNYTVCQNTLCCHLSYKMSEKRTDEVYVVGAFDGLHIAEGEYYLQICTLLKCLTTLIETCGQPVENASTWFDMFSLSGTFDSPHVFPELLLSGLQIAPTEFEILSDGRIINQNGTSAPVLMLTLFGRQYEKTKPPNASVDLETTSVMMITMFFVLYSTIW

SEQ ID NO: 58 - protein, 512 aa, Mus pahari
MNMSWWLACTAAFSVLCVLKASSLDTFLAAVYEHAVILPKDTLLPVSHSEALALMNQNLDLLEGAIVSAAKQGANIIVTPEDGIYGVRFTRDTIYPYLEDIPDPQVNWIPCDNPKRFGSTPVQERLSCLAKNNSIYVVANMGDKKPCNTSDSHCPPDGRFQYNTDVVFDSQGKLVARYHKQNIFMGEDQFNVPMEPEFVTFDTPFGKFGIFTCFDILFHDPAVTLVTEFQVDTILFPTAWMDVLPHLAAIEFHSAWAMGMGVNFLAANLHNPSRRMTGSGIYAPDSPRVFHYDRKTQEGKLLLAQLDSHPSHSPVNWTSYASSVESTPTKNQEFQSIVFFDEFTFVELKGIKGNHTVCQNDLCCHLSYQMSEKRADEVYAFGAFDGLHTVEGQYYLQICILLKCKTTNLRTCGSSVDTASTRFEMFSLSGTFGTRYVFPEVLLSEVKLAPGEFQVSSDGRLVSLKPTSGPVLTVGLFGRLYGKDWSSNASSDFIAHSLIIMLIMTPIIHYLC

SEQ ID NO: 59 - protein, 517 aa, Nematostella vectensis
MFRWFVAKFSLLALVIISHIKSVPESEFIAAVYEHVPPLNRPKEITRANALGIMMRNIDTYEEQMVIARDKNSSIIVFPEYGLTGWNQTRSVFKHFLENIPDPKISSNPCLDPGINKTTPILYRLSCLARKYAMYLVVNMGDIKPCQKASDPHCPGDGRYQYNTNVVFSDNGTLVARYHKQHPFMNEMKVVNRPRVPELVTFQTPFGKFGTFVCFDVLFQAPAVQLVTSVGIDHVVFPTAWFDVLPLFPAIGFHSSWARGIGVNFLAANTHVPSFANTGSGIYSSSGAREYYRSFSASGKLLIASLPKSPKKSPSNMTYVKTPGNTNDDVFYSDLFGDRFLFKELDKAEAIIRVCYPGSKSCCTLHYKMAAKLPDELYALGVFDGLHTLEGQYYLRVCTLIKCFGMSRSSCGSQVSSASTIFDSFALRAELNASHVFPAVVAHGVTLLRDEWDHDVPVKQVESKRRLSKGLLSATLFGRVYSLDGGKTSSTGRATCGPTFILTLRIVCIAMISVNLT

SEQ ID NO: 60 - protein, 510 aa, Bos taurus
MIMSQLLNYVAVLFFCVSRASSLDTFIAAVYEHAVILPNATLVPVSPEEALAVMNRNLDLLEGAVTSASKQGAHIIVTPEDGIYGFNFTRESIYPYLEDIPDPQVNWIPCNNPDRFGHTPVQQRLSCLAKDNSIYIVANIGDKKSCNASDPQCPPDGRYQYNTDVVFDTKGKLVARYHKQNLFLNEDQFNAPKEPEVVTFNTTFGKFGIFTCFDILFHDPAVTLVRDSRVDTILFPTAWMNVLPHLSAIEFHSAWAMGMRVNFLASNLHYPLKKMTGSGIYAPDSPRAFHYDMKTEEGKLLLAQLDSHPHPTPVVNWTSYASGVEAHSVGNQEFTGIIFFDEFTFLELKEIGGNYTVCQRDLCCHLSYKMSEKRSDEVYALGAFDGLHTVEGSYYLQICTLLKCKTTDLHTCGDSVETASTRFEMFSLSGTFGTQYVFPEVLLSEIQLAPGEFQVSNDGRLFSLKPTSGPVLTVTLFGRLYEKDSAPNTLSDLTTQALRLNPKTDAWKSK

SEQ ID NO: 61 - protein, 586 aa, Bulinus truncatus
MSLVLWVPGFSTVEEEKKSSRDIMTSSIVAVLHVTALLLMCVGGVFTSRSKFKAAVYGHAVILPEDPTKVVTRAEAVLNMNKNLDVYREQARKAREQDAEIIVFPEDGLYGFRFKRESIYPYLEYIPDPRVVSWSPCDDPRRHHDTEVQMSLSCMAKENSIYVVANMGDKQACDVLTDPKCPSDGRYQFNTNVAFGPDGTLLARYHKYHLFYELQFDTPELELIHFDTPFGRFGLMVCFDVLFYDPGIPLILNLNISNIVFPTAWMDALPLLSALGFHSSFARGLGVNFLSANIHLPDYRFHGSGIYGPDGAEISYYGAKSTSKPKLLVAEVNSLGNPKPNLNLSASFPFSNRLGELRDSTETCDSESDEFNLKRYFKMPNINDTFESVLFGDVFTFVSLDKPSGHLAVCQKNVCCDLQYSIDEQSFGRELFAFGAFDGLHTYEGQYYFQICTLVRCAAADNRTTCGSVTSTSETNFLHLSMSAEMQTDFIYPQIVLSNNDGELQLAEPQSWDFSDGYLSTKKGFNKTLLSAALFGRYYSRDGCISQEQTDSVEIKVKLGNDGSSSILSFPWILCISILECVLIFK

SEQ ID NO: 62 - protein, 547 aa, Caerostris darwini
MARSRALLFLILFVHSSCLFAKKDFYRAAVLELSQFTNISYPAADILRINLNIYDLAASTAAKHEADIIVFPESGLLPFEKPNREWLLDFIEDIPDPKKMLTNPCAEPDLFSDRPIITKLSCLARHNNIYVVANTADFKKCETDESCDKDNSNCTVCPKDGHFFYNTNVVFNREGTLIAKYYKRHLYFEPEMNTPHKPEDAYFDTDFGNFSTFICFDLMFKEPVKAIGNLSVLNVAYPTYWFDHTPLIFFATPYQQAWAMANKVNLLAANGHHPQTGSLGSGIYSSSKGALIYTHNPDGRSKLLISNVPISPRDDTIDTNNLNPMRFFIENGTIVAAKGEEKPIFKKECLNTVLGDGKPGDYRCGPTNVDQYEFKKLQGNKGKITTCSNKLCCWLDYDAHSMNEDYYFGVSGKDLNFYGEFSFGTEACFIARCESYEGKACTNFKLLSHTIFRKVEIRGNFTTKYILPFAVDSEMRLTNRDNWSLDESRIMYDNVNKKSSLLFFGVYGRLFEKDKDLYNQPDGAVRISCTGNWLIFGLLVSLFLFYF

SEQ ID NO: 63 - protein, 496 aa, Chrysochloris asiatica
MIASQLLAFVAVPVFCILKVSAMDTFIAAVYEHAVVLGNTSLTPVSREEALALMNQNLDLLEGAITSAAMQGAHIIVTPEDGIYGWHFTRDALYPYLEDIPDPQVNWIPCNDPNRFGHTPVQERLSCLAKNNSIYVVANIGDKKLCHTSDPQCPSDGRYQYNTNVVFDSEGKLVARYHKQNLFMGEVQFNSPKMAEVVTFNTTFGRFGIFTCFDILFHDPAVTLVKDFHVDTILFPTAWMNVLPHLSAVEFHSAWAMGMGVNFLAANIHHPMMKMTGSGIYAPDSPPAFHYDMKTKEGKLLLSSLNSHPYSSRVNWSSHASRMEAHSKGNQEFKNTVFFDEFTFVELMGIKGNYTVCQKDLCCHLSYKMTEKRSDEVYALGAFDGLHTVEGTYYLQICTMLKCKTTNIKTCGNSVETASTRFEMFSLSGTFGTQYVFPEVLLSEVQLAPGDFQVSSDGQLSSLKPTSGPVLTVTLFGRWYEKDQMANASSTTEHNH

SEQ ID NO: 64 - protein, 488 aa, Ptilonorhynchus violaceus
MLPFQTLLPTVVFAVTALQALASDTFIAAVYEHAVILPHPTGEPVSPDQALALMNQNMNVLEGAIKEAARQGAHIIVTPEDGIYGWRFTRESIYPYLEDIPDPVVNWIPCTDPSRFGPAPVQERLSCMARNNSIYVVANIGDKKPCNSSDPDCPRDGRYQYNTDVVFDPEGKLVARYHKYNLFMVETQFNYPKEPEAVTFETPFGKFGIFTCFDILFYEPAVVLVNKMQVDTVLFPTAWMNVLPFLTAVEFHSAWAMGMRVNLLAANTHKTILAMTGSGIYAPAGARTYSYNMETEDGHLLIAELDAHPRLSPASPPAVSWNSYALSVERFSQNDHEFTGIIFHDRFTFTELTKPEGNLTVCQKDLCCHLSYKMAGKRDDEAYVLGAFDGLHVVEGQYYLQICTLLKCQSTDLSTCGQPVETAQTKFEMFSLSGTFGTSYVFPEVLYSGVQLAPGEFEVLNDGRLISKAKPTKPVVTVTLFGRWYEKD

SEQ ID NO: 65 - protein, 544 aa, Nephila pilipes
MERDKVTVGNETSLLTGDEDDCLSDNVPQHPNENLETMIAALLSLTAFLAGELSSVKSDGDFYTAAVYEHARYDSATDSAQVIIQRNLANYTIATEIAKSKGADCIVFPEYGVFSPGERSQLKRFLEDIPDPKASRVNPCEESETYSDRPILYALSCLAKNNSIFVVANTGDIKKCEGEPDCPSDGDFHFNTNVVFDRTGTLLVRYHKEHLFYEFGMDLARETQQDTTFETDFGTFATYICFDIDYADMSRVARQPDVDGIMFPTMWVEQPPQMKSIQIWEAWAIANNSTILASNIQLPGYNALGSGIFHGSGPLTYTFNPDGISKLLVAKVPKSGSEPVSPDASITAITETRTWDWKDDGKDVGYECSSRVLGQATDIRTEYRCVEENVTDYSYTELEGPSGHFEICNNGLCCSLDYTTTGLFNRFFLGAFNGLYNHFGRYSWYEEDCILVRCDARGDRPCATFPMKSETVFTQVLLKGNFSSEIVFPSVLENDMRLTPKSDWRFERPNEQEAFIQFNSETGRKVITVGMKGRSYDKDPPYQR

SEQ ID NO: 66 - protein, 467 aa, Sylvietta virens
MLPSQALLPAVLLALAALQALASDTFIAAVYEHAVILPDATEEPVPPSQALALMNKNMDVLEGAVEEAARQGAHIIVTPEDGIYGWRFTRESIYPYLEDIPDPAVNWIPCTDPSRFGPAPVQERLSCMARNHSIYVVANIGDKKPCDSSDPDCPEDGHYQYNTDVVFDTQGKLVARYHKYNLFFESQFNYPKEPEAVTFETPFGKFGIFTCFDILFYEPAVVLVSKMQVDTVLFPTAWMNFLPFLTAVQLHSAWAMGMRVNVLAANTHNTSMEMTGSGIYAPAGAKTYFYNTKTEDGHLLIAELDAHPRLSPASPPAVSWNSYALSVERFSQNDHEFTGVIFEDPFTFMELTKPGGNVTVCQKDFCCHLRYKMAEKRDDEVYVLGAFDGLHVVEGQYYMQICTLLKCPSSNLSTCGGEHVETAQTKFEMFSLSGTFGTSYVFPEVLYSGVQLAPGEFEVTGHPSLLS

SEQ ID NO: 67 - protein, 444 aa, Poecile atricapillus
MLPSQVLLPAVLFALAALQALASDTFMAAVYEHAVILPGPTEEPVSPDSALTLMNKNMDVLEGAIKEAAQQGAHIIVTPEDGIYGWRFTRESIYPYLEDIPDPVVNWIPCTDPSRFGPAPVQERLSCMARNNSIYVVANIGDKKPCDSSDPDCPRDGRYQYNTDVVFDTQGKLVARYHKYNLFQGESQFNYPKEPEAVIFETPFGKFGIFTCFDILFYEPAVVLVSKMQVDTVLFPSAWMNVLPFLTAIEFHSAWAMGMRVNVLAANTHNTSMAMTGSGIYAPAGARTYSYNMKTEDGHLLIAELDAHPRLSPASPPAVSWNSYALNVGRFSQNDHEFTGIIFEDPFTFTELTKPGGNLTVCQKDLCCHLTYKMAEKRDDEVYVLGAFDGLHVIEGQYYLQICSLLKCLSTNLSTCGQPVEMAQTKFEMFSLSGTFGTSYVFPE

SEQ ID NO: 68 - protein, 441 aa, Araneus ventricosus
MSYAEYIPDPKVQKFNPCVENTKDRTILYTVSCLARENNIYIVANLVGSEPCETTCDAAEVENCDAKCPDDGVFFYNTDVAFDREGNLIARYRKVHPYYREIVNVPVETEVGIFETEFGKFGMIVCFDLVFKEAVLLKDEYDIDTLLFPTYWYDDIVPLNAVEFQQAWAIGNGVNVLSANTHAPGTGSMGSGIFSKDDGALVYTYEPDGKSKLLLANVGIKGDSEIPNISSLIEITPDSAVPITETGEKFPVHCYDKVAGPALNGEIEYRCLKLKLENYSLTKLEKPSGEVQACNNELCCTLSYSTQNLQDEYYLAVYNGLNDAFDYFHWWEEICVLTRCDPFGRNPCALQPSISKTVFQKVELKGNFKSEKVYPTVSKTNNRLAPKSEWNFEQGETKAVLSFQSTLNLPLLKVGLLGRSYDRDPEFIPYFPKEFKKQQIN

SEQ ID NO: 69 - DNA, 1542 bp, synthetic construct
  source 1..1542: mol_type = other DNA
atgatcacctctccgttgctggcttatgttgcgattctgttcttgtgtgtcctgaaagccagctccctggatacctttatcgccgctgtttatgaacacgcggtgattcttcctgatgcccccttgacaccagtatcccacgaagaggctctcatgctgatgaaccgcaacctggatcttttggaaggcgcaatcatgtcagcagcaaagcaaggtgcgcacatcattgtgactccagaagatggtgtttacggcttcttcttcagccgcgaatctatctactcctacctcgaggacatcccagagccgcacgtgaactggatcccttgcaccaatcccaaccgctttggccacaccccagtacaaaagcgactcagctgcctggcccgggacaactccatctacattgtggccaatatcggagacaagaagccatgcaacgcctcggaccccgactgtcctcatgacggccgttaccagtacaacaccgatgtggtctttgattccgagggccgactggtcgcacgctaccacaagcaaaatcttttcttgggtgaagatcaattcgatgcacccaaggagcccgagatcgtgacgttcgacaccacctttggccgttttggcatcttcacgtgctttgatattctcttccatgatcctgctgtgaccctggtcaaagatttccaggtagacaccatcctcttcccgaccgcgtggatgaacgttctcccacatttgaccgctattgaattccactcggcttgggctatgggcatgcgtgtcaacttccttgcggccaatatccacttcccgctgcgaaaaatgacaggatcgggcatctatgcacctgattctccacgcgcctttcattacgatatgaagaccaaggaaggaaaactcctcttgtcacaattggattcccacccgcaccgcccagcagtgaattggacgtcttatgcctcaggtttgccagcccccttggtgggtaaccaggaattcaaatccattgtcttttttgacgagttcaccttcttcgagcttaaaggtgttgcgggaaactacacagtttgccagaaggatctctgctgccagctttcttaccggatgcttgagatgcgtgaagacgaagtttatgcgctcggcgcattcgatggacttcatactgtggaaggtagctattacttgcagatttgcacattgctgaagtgtaaaaccatggacctgcactcctgtggtgactcggtcgaaactgcttccacccgctttgaaatgttctccctcagcggcacgtttggcacccagtatgtgttccctgaggtgttgctgtcggacattcagcttgcgcctggagaatttcaggtttcatccgatggccgcttgttcagcctgaagccaccgtctggaccggtccttactgtcactctgtttggccgcctctacgagcgtgattctgcatcaggtgcttcagcagacctcgtggcacaaggactgcgagtcatgctcggtgttatcattactatcatgtactccttgtcctggtaa

SEQ ID NO: 70 - DNA, 1563 bp, Bos taurus
  source 1..1563: mol_type = genomic DNA
atgatcacttcctctttacaaacctctgtggcagtttttgccctagtaattctacatgtcagcactctggatacttttaaagctgcagtgtatgaacatgctgtcatactgccaaatgacacagaaacacctgtttctccggatgaggccttgttcctcatgaacaagaacatagatattctggagaaggcaattaagcaggcagctgagcagggtgctcagattatcgtgaccccagaagatgcactttatggatggaaatttaccagggaaaccattttcccttatctggagaatatcccagatcctcaggtgaactggattccatgtcaagaaccccacagatttggtcatacaccagtacaagcaagactcagctgcctggccaagaacaactctatctatgtcgtggcaaatatgggtgacaaaaagccatgtaattcccataataccacttgtccttctaatggccattatcaatacaacaccaacgtggtgtatgatgcaaagggaaagctggtggcgcgttaccataagtaccacctctaccgtgagattcagtttgatgtccccgaaaagccagagttggtgactttcaacaccacctttggaaagtttggcattttcacgtgctttgatattctcttccatgatcctgctgtgaccctggtgaaagattcccacgtggacaccatattgtttcccacagcttggatgaacgttttgccctttatgacagctattgaatttcattcggcttgggcaatgggaatgagagttaatcttcttgcagcaaacacacataatgtcagcctaaaaatgacaggcagtggtatctatgcaccacactctcccaaagtctaccatcatgatatggaaacagagtcaggaaagatcctctttgcagaggtggattcccatccccgaaactcctcgacctacccaccagccatgaattggagtgcctacgccacctccatcacaccctttccaggacctaaaaattccttcaggggatttatttcccgtgatgagttcaactttacaaaactctctgaaagtgcaggaaaccttacagtctgtcaaaaagagctctgctgtcatttaagctacagaatgttagcaaaggagggggatgaagtgtatgttctaggagcttttgcaggacttcatgggagaaggagaagagaatattggcaggtatgcacaatgctgaaatgcagatctactgatgtgacaacttgtggacagccagtagaaactgctttgacgagatttgaaatgttctctctaagtggcacatttggaacagagtatgtttttcctgaagtgctacttactaaaatcaagctggcacctgaaaaatttgaggtactagaagatgggcgtttggtaaacaagaatggattatctgagcctgtcctaacagtgtcgctctttgggagatggtacaccaaggacgcacattccatctcaggcgggaccagcaactcagcaacagcgtacctgatgatacccacattattaatgatcataaccttgcgggatgctgtgatggtgtag

SEQ ID NO: 71 - DNA, 1563 bp, Ovis aries
  source 1..1563: mol_type = genomic DNA
atgatcacttcctctttacaaacctctgtggcagtttttgccctaataattctacatgtcagtgctctggatacctttaaagctgcagtgtatgaacatgctgtcatactgccaaacgacacaaaaacacctgtttctccggatgaggctttgttcctcatgaacaagaacatagatatcctggagaaggcaattaagcaggcagctgagcagggtgctcagatcattgtgaccccagaagatgcactttatggatggaaatttaccagggaaaccattttcccttatctggagaatatcccagatcctcaggtgaactggattccatgtcaagacccccacagatttggtcatacaccagtacaagcaagactcagctgcctggccaagaacaactctatctatgtcgtggcaaatatgggtgacaaaaagccatgtagttcccgtaataccacgtgtccttctaatggccattatcagtacaacaccaacgtggtatatgatgcaaagggaaagctggtggcgcgttaccataagtaccacctctaccatgagcttcaatttgatgtccccgaaaagccagagctggtgactttcaacaccgcctttggaaagtttggcatcttcacgtgctttgatattctcttccatgatcctgctgtgaccctggtgaaagatttccacgtggacaccatattgtttcccacagcttggatgaacgttttgccctttttgacagctattgaatttcattcggcatgggcaatgggaatgagagttaatcttcttgcagcaaacacacataatgtcagcctgaaaatgacaggcagtggtatctatgcaccacactctcccaaagtctaccatcatgatatggaaacagagtcaggaaagatcctctttgcagaggtggattcccatccccgaaactcctcgacctccccaccagccgtgaactggagtgcctacgccacctccatcacaccctttccaggacctaaaaattccttcaggggatttatttcccgcgatgagttcaacttcacaaagctctctgaaagtgcaggaaaccttacagtctgtcaaaaagagctctgctgtcatttaagctacagaatgttaggaaaggaggaggatgaagtgtatgttctaggagcttttgcaggacttcatgggagaaggagaagagaatattggcaggtatgcacaatgctgaaatgcagatctactaatttgacaacttgtggacagtcagtagaaactgctctgacgagatttgaaatgttctctctaagcggcacatttggaacagagtatgtttttcctgaagtgctacttactaaaatcaagctggcacctgaaaaatttgaggtactagaagatgggcgtttggtaaacaagaatggattatctgaacctgtcctaacagtgtcgctctttgggaggtggtacaccaaggacatacattccatctcaggcgggaccagcaactcagcaacaacgtacctgatgatacccacattattaatgatcatagtcttgcagtatgctgtgatggtgtag

SEQ ID NO: 72 - DNA, 1563 bp, Balaenoptera sp.
  source 1..1563: mol_type = genomic DNA
atgatcacttcctcttttcaaacttccgtggcagttttggccctaattattctgcatgtcagcgctttggatactttcaaagctgcagtatatgaacatgcggtcatattgccgaatcacacagaaacacgtgtttctcaggatgatgccttgctcctcatgaacaagaatatagatattctggagatagcaattaagcaggccgctgaacagggtgctcagatcattgtgactccagaagatgcaatttatggatggaaatttaccagggaagccattttcccttatctggaggatattccagatcctctggtgaattggattccatgtcaagacccacacagatttggtcgtacaccagtacaagcaagactcagctgcctggccaagaacaactctatctatgtcttggcaaatattggggacaaaaagccatgtaattcctgtgactccacgtgtcctcctaatggccattatcaatacaacaccaacgtggtgtatgatgcaaagggaaagctggtggcacgttaccataagtacaacctctactctgagcttcagtttgatgtccccaaaaagccggagttggtgactttcaacacctcctttggaaagtttggcattttcacatgctttgatattctcttccatgatcctgctgtgaccctggtgaaagatttccacgtggacaccatattgtttcccacagcttggatgaatgttttgccctttttgacagctattgaatttcattcagcttgggcaatgggaatgagagttaatcttcttgtggccaacacacataatgtcagcctaaagatgacaggcagtggtatctatgcaccacactcgcccaaagtatatcattatgatatggaaacagagctgggaaagatcctctttgcagaggtggattcgcatccccgaaactctctgacctacccaccagctgtgaattggagtgcctacgccaccaccatcaaaccattcccaggacagaaaaactcctttaggggctttatttcccgggatgagttcaacttcacagaactctttgaaaatgcaggaaaccttacagtctgtcaaaaagagctgtgctgtcatttaagctacagaatgttaggaaaagaggagaatgaagtgtatgttctaggagcttttgctggacttcatggccgaaggagaagagagtattggcaggtatgcacaatggtgagatgtaaatctactaatctgacaacttgtggacagccagtagacactgctctgacaagatttgaaatgttctccctaagtggcacatttagaacggagtatgttttccctgaagtgctacttactaaaatcaatctggcacctggaaaatttgaggtactaaaagatgggcgtttggtaaacaagaatggatcatctgagcctatcctaacagtgtcactgtttgggagatggtacactgaagacgtacgttccatctcaggcaggaccagcaactcagcgacaacttacctgataatatccacattattaatgatcatagctttgcaaaatattgtgatggtgtag

SEQ ID NO: 73 - DNA, 1566 bp, Sus scrofa
  source 1..1566: mol_type = genomic DNA
atgatcgcttcctcttttcaaactacgttagcagtttttgccctaattatcctgcatgtccgtgcactggatacttttaaggctgctgtatatgaacatgctgtcatcctgccaaatgacacaaaaacacctgtttcttgggatgatgctttgctcctcatgaacaagaatatggatattctggagagagcaattaagcaggcagctgagcagggttctcaaatcattgtgactccagaagatgcactttatggatggaaatttaccagggaaaccatttttccctatctggaggatgtcccagatcctcagaagaactggattccatgtcaagacccccgcagatttggtcacacaccagtgcaagtaagactcagctgcctggccaagaataactctatctatgtcttggcgaatattggagacaaaaagccctgtaattcccatgattccaaatgtcctcctaatggctattatcaatacaacaccaacgtggtatatgattcagagggaaagctggtggcacgttaccataagtaccacctctaccatgagcttcagtttgatgtccctgaaaagccggaggtggtgactttcaacaccccttttggaaagttcggcattttcacgtgcttcgatattctcttccgtgatcctgctgtgacgctggtgcaagatttccacgtggacaccatattgtttccggcagcttggatgaatgtgttgccccttttgacagctattgaattccattcggcttgggcaatgggcatgagagttaatcttcttgcagccaacgtacatcatgtccgcctaaatatgacaggcagtggtatctatgctccacattctcccaaagtataccattatgacatggaaacggactcgggaaaaatcctccttgcggaggtggatgcgcatccccgcacctccccgacctacccaacagccgtgaactggggtgcctacaccctaagcatccagccgttcccaatactgcaaaactccttcaggggattcatttcgcgggatgagttccacttcacagaactctctgaaagtgcaggaaaccttacggtctgtcataaagagctctgctgtcatttaagctacagaatgctagcgaaggaggagaatgaagtgtatgttctaggagcttttacaggacttcatggccgaaggaganaggagtattggcaggtatgcacaatgttgaaatgcaaatctactaatctgacaacttgcggacagcctgtagaaactgctctgacgagatttgagttgttctctctaagtggtacatttggaacagagtatgtttttcccgaagtgctccttactaaaatcaatctggcacctggaaaatttgaggtactgaaagatgggcgtttggtaaacaagaacggaacatctgagcctgtcctaggagtgtcactgtttgggagatggtacattagggatgtaccactttccatctcgggaggaccagcacctcagcaaaaaatgtacctgataatttccgcattattaatgatcatagctttgcaaaatattgtcatggtatag

SEQ ID NO: 74 - DNA, 1563 bp, Echinops telfairi
  source 1..1563: mol_type = genomic DNA
atgactgctgcttctttccaaacctccgcagcagtgtttgccctacttaccctgcatgtgggtgccctgaatactttcacagccgcggtgtacgaacatgccgtcatactgccaaataaaacactgacaccggtttctcagcaagatgccttggccctgatgaacaagaatatagatatcctggaggaagttattaagcaggcagctgagcagggcgctcaaatcattgtgactcctgaagatgctctctatggatgggaatttggatgggaaactgtcttcccttttttggaggatgtcccagcccctgaggtggactggattccatgccgagaccctcacagatttggtcacacaccagtacaaacaagactcagttgcatggccaagaacaactctatctatgtcgtggcaaatattggggacaaaaagaagtgtaactcccaggactccacctgcccttccactggctattttcagtacaataccaatgtggtgtacaacgcggaggggaaactggtggctcgttatcataagcaccacctctacggtgagtttcaatttaatgccactagaaagccagaagtggtgacattcgacaccacctttggaaagtttggtattttcacctgctttgatatactcttccgtgagcctgctgtgcgactggtggaagagtcccaagtcgacaccatattgttcccaacagcttggatgaatagcttaccctttctgacagctattgaatttcattcagcttgggcaatgcaaatgagagtgaatcttcttgctgctaacctacatcataccagtaaaaggatgacagggagtggcatctatggcccgaagggccccagcgtgtatcactatgatatggaaacggcatcgggacagctcctcctctcccaggtggactcccacccccggcgctcccctgactaccccgcagctgtcgactgggacgcctatgccaggagcatcaaaccattcccaacacagaagcatgcctttgggggcaacgttcacgctgaccagttcaacttcaccgagctgtatgaacgtgcaggaaaccttactgtctgccagaaagagctctgctgtcatttaagctacagaatgttaggaaaagaagagagtgaagtctatgttctaggagctttcacgggactccacgggaaaagggcgaaagagtactggcaggtctgcacgttgctgaaatgcaaaacgactatcttgacatcttgtggagaggtggtagaaactgcttctacaagatttgaaatgttctctctaagtggcaccttcgaaaccgagtatgtttatcccgaagtgttacttagtaaaattcagctgtcacctggaaaatttgaggtactaaaagatggacgtttggtagacaagaatgggacatctggacctattgtaaccgtgacactgtttggaaggtggtacgaaagggacagacttcagaattcagccaagaccagcaattcagcaatacacttcctactgatcgccacatcattcatgatcacagctttgcaaaatattgccatgacatag

SEQ ID NO: 75 - DNA, 1563 bp, Oryctolagus cuniculus
  source 1..1563: mol_type = genomic DNA
atgatcacttcctcttttccaacccgcgtggcagtttttgccctgatcagcctgcgtgtcggtgctctggacacttttgtagctgcagtgtatgaacatgccgtcgttttgccaaagaaaacagaaacgcctgtttctcaagacgatgccttgcttctcatgaacaagaatatagatattctggagagtgcgattaagcaggcagcagggaagaatgctcgaatcattgtgactccagaagatgcactttatgggtggaaatttaccagagaaacaattttcccttatctggaggatatcccagatcctcaggtgaactggatcccatgtcaagacccccacagatttggtcacacaccagtacaagcaagactcagctgcctggccaaaaacaattctatctatgtcttggcaaatattggagacaagaagccttgtaattcccatgactccacatgtcctcataatggctattatcaatacaataccaatgtggtgtacgattcagagggaaaactggtggcacgttatcataagtaccacctatactctgaacctcagtttgatgttcctgagaaaccagagctggtaactttcaacaccacatttggaaagtttggcattttcacatgttttgatatactcttccatgatcctgctgttaccttggtgaaagatttccatgtggacaccatattgctccctacagcttggatgaatgttctgcccctcttgacagctattgaattccattcagcttgggccatgggaatgagagttaatcttcttgtggccaatacacatcatgtcagcttaaatatgacagggagtggcatctatgtaccacattctcccaaagtgtaccactatgacatggaaacagacttgggaaagctcctcctttcagaggtggattcacatccccaaacctcttcttcctacccaacagctgtgaattggagtgcctatgccatggccatcagaccattctcagtacagaaagacactttcagaggatttatttctcgggacgagttcaattttacagaactttttgaaaatgcaggaaatattacaatttgtcaaaaagagctctgctgtcatttaagctacagagtgctagaaaaagaagcagatgaaatttatgtcctaggagcctttacaggatttcatggccgaaggaggagagaatactggcaggtatgcacaatgttgaaatgtaaaactactaatctgaccacatgtggacggccagtagagactgcttctacacgatttgaaatgttctccctcagtggcacatttggaacagagtatgtttttcctgaagttctactcactggaattcatctatcacctggaaaatttgaagtgctaaaagatgggcgtttggtaaataagaatggattatctgggcctatcctaacagtgtcactgtttgggaggtggtacacaaaggacacactttacaaatcatctgggaccaacaattcagcaataccttacctgctaatcttcattgtcctaatgactgcagattttcaaaatattgtattgctgtaa

SEQ ID NO: 76 - DNA, 1563 bp, Lepus europaeus
  source 1..1563: mol_type = genomic DNA
atgatcacttcctcttttccaacccatgtggcagtttttgccctgatcagcctgcgcgtcggtgctctggacacttttgtagctgcagtgtatgaacatgccgtcattttgccaaagaaaacagaaacgcctgtttctcaagacgatgccttgctcctcatgaacaagaatatagatattctggagagtgcgattaagcaggcagcagggaagaatgctcgaatcattgtgactccagaagatgcactttatgggtggaaatttaccagagaaacgattttcccatatctggaggatatcccagaccctcaggtgaactggatcccatgtcaagacccccacagatttggtcacacaccagtacaagcaagactcagctgcctggccaagaacaactctatctatgtcttggcaaatattggagacaagaagccttgtaattcccatgactccacatgtcctcataatggctattatcaatacaataccaatgtggtgtacgattcagagggaaaactggtggcacgttatcataagtaccacctatactctgagcctcagtttgatgttcctgagaaaccggagttggtaactttcaacaccacatttggaaagtttggcattttcacatgttttgatatactcttccatgatcctgctgttaccttggtgaaagatttccatgtggacaccatattgttccccacagcatggatgaatgttctgcccctcttgacagctattgaattccattcagcttgggccatgggaatgagagttaatcttcttgtggccaatacacatcatgtcagcctaaatatgacagggagtggcatctatgcaccacattctcccaaagtgtaccactatgacatggaaacagacttgggaaagctcctcctttcagaggtggattcacatcccaaaacctcttcttcctacccaacggctgtgaattggagtgcctatgccatggccatcagaccattctcagtacagaaagacactttcagaggatttatttctcgggacgagttcaattttacagaactttttgaaaatgcaggaaatattacaatttgtcaaaaagagctctgctgtcatctaagctacagaatactagaaaaagaagcagatgaaatttatgtcctaggagcctttacaggatttcatggccgaaggaggagagaatactggcaggtatgtactatgctgaagtgtaaaactactaatctgaccacatgtggacagccggtcgagactgcttctacacgatttgaaatgttctccctcagtggcacatttggaacagagtatgtttttcctgaaattctactcactgaaattcatctatcacctggaaaatttgaagtgctaaaagatgggcgtttggtaaataagaatggattatctgggcctatcctaacagtgtcactgttcggaaggtggtacacaaaggacacactttacaaatcatctgggaccagcaattcagcaataccttacctgctaatcttcattgtcctaatgattgcagatttgcaaaatattgtattgctgtaa

SEQ ID NO: 77 - DNA, 1548 bp, Dasypus novemcinctus
  source 1..1548: mol_type = genomic DNA
atgatcacgtcaccttttctaccttcggcagtagttttggccctaatcattctgaatgtcggtgcactgggcacttttaaagctgccgtgtacgagcatgctgtgatattgccaaaccaaacactcagagttgtttctcaggaagatgcgctgctcttgatgaacaagaatctagatatcctggaggcagagataaagcaggcagctcagcagggtgctcaaatcattgtgactccagaagatgccctttatggttggactttcaccagggagactgttttcccttatctggaggacatcccagacccccaggagaactggattccatgtcagaacccccagagatttggtcacacaccaatacaaaaaagactcagctgcctggccaaggacaactctatatatatcctggcaaatattggggacaaaaagcagtgcgattcccggaactccgcctgtcctcgcaatggttattatcaatacaataccaacgttgtgtatgataaagagggaaaactggtggcgcgttaccataagtaccatctctacaatgagcctcagtttaacgcccctaaaaggccggaattggtgaccttcaacactgattttggaaagtttggcattttcacgtgctttgatatactctttaaagagcctgcagtgaccctggtggaagaattacaagtggacaccatactgtttgccacagcttggatgaatgtcttgccctttttgacagctattgaattccactcggcttgggcaatgggaatgagagtcaatgttcttgcagccaacatacacaatcccagcctaaatatgacagggagtggtatctactcaccacattcccccaaagcataccattatgatatgaaaacaaagtttggaaggctccttctttcagagttggattcacatccccgaggttctgtatcttacccaccagctgtgagctggacaacctacgccaagtccatcaaaccattcccagtacagaaacccgccttcctgagaagtatttccagagacgagttcaacttcacggaactttttgaaagtgcaggaaatcttacagtctgtcaaaaagagctctgctgtcatttaagctacagaatgttagaaaaagacacaaaagaatcctacgttctcggagcatttacaggacttcacggcagaaggcaacaagagtactgggaggtatgtacaatactgaaatgtgaagctaataatttcatgacttgtggacggacaggggaaactgcttctacaagatttgaaatgttctccctaagtggcacatttaaaacagagtatgtctttcctgaagtgttacttgataaaattcacctgtcacccggaaaatttgaggtactaaaggatggccgtttggtaaacaagaatgggacatctgggcagatcctaacagtgtcactgtttggcaggtggtatgccaaggatgagctttccaactcaggcaagacccacagttcagcagtgccaacgctggcattagtaatgatagtagctttgatgtatgatgtgtga

SEQ ID NO: 78 - DNA, 1404 bp, Camelus dromedarius
  source 1..1404: mol_type = genomic DNA
atgaacaagaatatggatattctggagaaagcaattaagcaggcagctgagcagggtgctcaaattattgtgactcctgaagatgccatttatggctggaaatttacccgggaaaccattttcccttatcttgagtatatcccagaccctcaggtgaactggattccatgtcaacaaccccacagatttggtcacacaccagtacaagtaagactcagctgcctggccaagaacaactctatctatgtcttggcgaatattggggacaaaaagccatgtaattccaggggctccacgtgccctcctagtggctattaccaatacaacaccaacgtagtgtatgatacacagggaaagctggtggcacgttaccataagtaccacctctactctgagcctcagtttgatgtccctgaaaagccagaggtggtgactttcaacaccaccttcggaaggtttggcattttcacgtgcttcgatatactcttccatgatcctgctgtgaccctggtgaaagatttccaggtggacaccgtattgtttcccacagcgtggatgaatgttttgccccttttgacagctattgaattccattcagcctgggctatgggcatgggagttaatcttttggtggccaacacacatcacgtcagcctaagaatgacaggcagcggtatctatgcaccacactctcccaaggtctattattatgataaggaaacagagttgggaaagcttctccttgcagaggtggactcgcatccccgaatctctccgacctacccaacagccgtgaattggagcgcctacgccacaaccatcaaaccattcccagtacagaagcacactttcgggggatttatttcccgggatgagttcaacttcacagagctctttgaaaatgcaggaaacctgacagtctgtcaaaaagagctctgctgtcatttaagctacagaatgctgggaaaagaggagaatgaagtgtatgtgctaggagcttttacaggacttcatggccgaaggagaagtgaatattggcaggtatgcaccctggtgaaatgcaaatctactaacttgacgacttgtggacagccagtagaaactgctctgacaagatttgaaatgttctccctaagtggcacatttggaacagagtatgtttttcctgaagtgctacttactaatatccatctggcacctggaaaatttgaggttctaaatgatgggcgtttggtaaacaagaatggattatctgagcctatcctaacagtgtcactgtttgggaggtggtacactaaagacgcactctccatctcaggtgggaccagcaacgcagcaacaacttacctgttaatattcacatcattaatgatcatagctttacaaaatactgtgatggtataa

SEQ ID NO: 79 - DNA, 1380 bp, Homo sapiens
  source 1..1380: mol_type = genomic DNA
atggtcacttcctcttttccaatctctgtggcagtttttgccctaataaccctgcaggttggtactcaggacagttttatagctgcagtgtatgaacatgctgtcattttgccaaataaaacagaaacaccagtttctcaggaggatgccttgaatctcatgaacgagaatatagacattctggagacagcgatcaagcaggcagctgagcagggtgctcgaatcattgtgactccagaagatgcactttatggatggaaatttaccagggaaactgttttcccttatctggaggatatcccagaccctcaggtgaactggattccgtgtcaagacccccacagatttggtcacacaccagtacaagcaagactcagctgcctggccaaggacaactctatctatgtcttggcaaatttgggggacaaaaagccatgtaattcccgtgactccacatgtcctcctaatggctactttcaatacaataccaatgtggtgtataatacagaaggaaaactcgtggcacgttaccataagtaccacctgtactctgagcctcagtttaatgtccctgaaaagccggagttggtgactttcaacaccgcatttggaaggtttggcattttcacgtgctttgatatattcttctatgatcctggtgttaccctggtgaaagatttccatgtggacaccatactgtttcccacagcttggatgaacgttttgccccttttgacagctattgaattccattcagcttgggcaatgggaatgggagttaatcttcttgtggccaacacacatcatgtcagcctaaatatgacaggaagtggtatttatgcaccaaatggtcccaaagtgtatcattatgacatgaagacagagttgggaaaacttctcctttcagaggtggattcacatcccctatcctcgcttgcctacccaacagctgttaattggaatgcctacgccaccaccatcaaaccatttccagtacagaaaaacactttcaggggatttatttccagggatgggttcaacttcacagaactttttgaaaatgcaggaaaccttacagtctgtcaaaaggagctttgctgtcatttaagctacagaatgttacaaaaagaagagaatgaagtatacgttctaggagcttttacaggattacatggccgaaggagaagagagtactggcaggtctgcacaatgctgaagtgcaaaactactaatttgacaacttgtggacggccagtagaaactgcttctacaagatttgaaatgttctccctcagtggcacatttggaacagagtatgtttttcctgaagtgctacttaccgaaattcatctgtcacctggaaaatttgagcaaacctga

SEQ ID NO: 80 - DNA, 1560 bp, Globicephala melas
  source 1..1560: mol_type = genomic DNA
atgatcatttcctcttttcaaacctccgtggcagtttttgcgctaattattctgcatgtcagtgctttggctactttcaaagctgcagtatatgaacatgctgtcatattgccaaatgacacagaaacacccgtttctcaggattatgccttgctcctcatgaacaggaatatagatattctggagagagcaattaagcaggctgctgaacagggtgctcagatcattgtgactccggaagatgcaatttacggatggaaatttaccagtgaagccattttcccttatctggaggatattccagatcctctggtgaattggattccatgtcaagacccacacagatttggtcatacaccagtacaagcaagactcagctgcctggccaaggacaactctatctatgtcgtggcaaatatcggggacaaaaagccatgtcattcccgtgactccgcgtgtcctcctaatggccattatcaatacaacaccaacgtggtgtatgatgcaaatggaaagctggtggcacgttaccataagtacaacctctactctgagcttcagtttgatgtccccgaaaagccagagctggtgactttcaacacctcctttgggaagtttggcattttcacatgctttgatatcctcttccatgatcctgctgtgaccctggtgaaagatttccacgtggacactatattgtttcccacagcttggatgaatgttttgccctttttgacagctattgagtttcattcagcttgggcaatgggaatgagagttaatcttcttgtggccaacacacataatgtccgcctaaaaatgacaggcagtggtatctatgcaccacactctcccaaagcgtaccattatgatatggaaacagagttgggaaagatcctctttgcagaggtggattcgtatccccgaaactctccaacctacccaccagctgtgaattggagtgcctacgccaccaccatcaaaccattcccaggacagaaaaattcctttaggggctttatttcctgggatgagttcaacttcacagaactctttgaaaatgcaggaaaccttacagtctgtcaaaaagagctgtgctgtcatttaagctacagaatattaggaaaagagaatgaagtgtatgttctaggagcttttgctggacttcatggcagaaggagaagagagtattggcaggtatgcacaatggtgaaatgtaaatctactaatctggcaacttgtggacagccagtagacactgctctgacaagatttgaaatgttctccctaagtggcacatttagaacggagtatgtttttcctgaagtgctacttactaaaatcaatctggcacctggaaaatttgaggtactaaaagatgggcgtttggtaaacaagaatggatcatctgagcctatcctaacagtgtcactgtttggcagatggtacactaaagacgtacgttccatctcaggcgggaccagcaactcagcaacgacttacctgataatatccacattattaatgatcatagctttgcaaaatattgtgatggtgtag

SEQ ID NO: 81 - DNA, 1542 bp, Pteronotus mesoamericanus
  source 1..1542: mol_type = genomic DNA
atgcaatttcatttctgggagagaggccaggctaggacgtccttcaggctgtggcagagcccaaggctcctcctcaccatcctgagttcctcggtgtctttgtcatgtctccattgcgtggttgacgtgaatctgctgtgtccttctcggcaagatccaggagttggtgcccaggagaaccttgtgaaggatataaagcagggtgctcaaattattgtgactccagaagacgcaatttatggatggaattttaccagggaaaccattttcccttacctggaggatatcccagaccctcaggtggactggattccatgtcaagacccccacagatttggtcacacgccagtacaagaaagactcagctgcctggccaagaacaactctatctatgttttggcaaatattggagacaaaaagccatgtaattctaatgacttcacatgtcctcctaatggctattatcaatacaatactaatgtggtatatgataaaaatggacacctggtggcacgttaccacaagtaccacctctattctgaacctcagtttgatgtgcctgaaaagccggagctggtgactttcaacaccacctttggaaggttgggcattttcacctgcttcgatatactcttccacgatcctgctgtgaccctggtgaaggatctccacgtggacaccatactgtttcccacagcttggatgaatgtcttgccccttttaacagctattgaattccattcagcttgggcaatgggaatgagagtcaatcttcttgtggccaatacacatcatgtccgcctaaatatgacaggcagtggcatttatgcaccacagtctcccaaagtataccactacgatatggacacggagctgggaaagatccttctttcagaagtggattcgcatcctcgaagctcacctgcctacccagcggctgtgaactggagtgactatgccacgaccatcaaaccattcccagtacagaaaaacactttcaggggatttatttcccgggatgcgttcaacttcacagaactttctgcaattgcaggaaaccttacagtctgtcaaaaagatctctgctgtcatttaagctatggaatgttaagaaaagaagagaatgaagtatacgctctaggagctttctcaggacttcatggccgaaggagaagagagtattggcaggtatgcacaatgctgaaatgcaaaacaactaatttgacaacttgtggacaaccagtagaaactgctgtgacaagatttaatatattctccctaagtggcacatttggaacagagtatgtttttcctgaagtgctacttactaaaattcatctgtcacctggaaaatttgaggtactaaaagatgggcgcttgataaacaagaatggaacatctgagcctatcctaacagtgacactctttgggaggtggtacgctaaggactcacattccgtctcaggagggatcagcaatttggaaacaacttacctgttaatatccactttatcaatgatcatagctttacaaatattgtga

SEQ ID NO: 82 - DNA, 1554 bp, Equus caballus
  source 1..1554: mol_type = genomic DNA
atgattgcttcctcttttcgaacctctgcggctgtttttgccgtaattatcctgcatgttggggccctggatactttcatagctgcagtttatgaacacgccgtcatactgccaaaaacaacagtgacacctgtctctcaggacgatgccttgctcctcatgaacaagaatatagatattctggagagagcgattaagcaggcagcaaagcagggtgctcaaatcattgtgactccagaagatgccctttatggttggacatttaccagggaaaccattttcccttatctggaggacgtcccggaccctcaggcgaactggattccgtgtcaggactccaacagatttggttacacaccagtacaagtaagactcagctgcctggccaagaacaactcaatctatgtcttggcaaatatgggggacaagaagccatgtaatcgccataactccatgtgtcctcccaatggctattatcaatacaacaccaatgtggtatatgatgcggaggggaaactggtagcacgctaccataagtaccacctctactctgagcctcagtttgatgtccctgaaaagcccgagttggtgactttcaacaccacctttggaaggtttggcattttcacgtgctttgatattttcttccacgatcctgctgtcaccctggtgaaagatttccatgtggataccatcctgtttcccacagcttggatgaatgtcttgccccttctgacagctattgaattccactcggcttgggctgtgggcatgagagttaatcttcttgcggccaacacccacaatatccacatgaatatgacaggcagcggtatctatgcaccacagtctcccaaagtataccaccatgacatggagacagagtcgggaaagctcctccttgcggaagtggattcgcatccccgagactctcctgcctacccaacagctgtgaactggagtgcccatgccacaaccatcagaccattcccaggaaagaaaaacaccttccgggcatttatttcccgggacgctttcaacttcacagaactttttgaaaatgcaggaaaccttacagtctgtcaaaaagatctctgctgtcatttaagctacaggatgttaggaaaggaagaggatgaagtgtatgctctgggagctttttcaggacttcacggccgaaggagaaaagagtactggcaggtgtgcacaatgctgaaatgcaaaactactaatttgacaacttgtgggcggccggtagaaactgcttctacaagatttgaaatgttctccttaagtggcacatttggaacagagtatgtcattccagaagtgctacttactaaaattcacctgtcacctggaaaatttgaggtactacaagatgggcgtttggtgaacaagaacggaccatccgagcctatcctgacagtgtctctctttgggagatggtatgctaaggattcatcttccatcccaggcggaaccagcaattcagcaacaacttacctgctgatacccattttattgatgatcatagcttggcagatactgtga

SEQ ID NO: 83 - DNA, 1332 bp, Jaculus jaculus
  source 1..1332: mol_type = genomic DNA
atgggcgctcagatcattgtgactccagaggatgcactttatggatggaagtttactagggaaagcattttcccttacttggaagatgtccccgaccctcaggtggactggattccatgtcgagatccccacagatttggtcacacacccatacaagaaagactcagctgcctggccaagaacaactctatctatgtcgtagcaaatattggagacagaaagccatgcaagtcccatggatgccctcataatggctactatcagtacaatactgatgtggtatacgataaagagggaaaactcgtggcacgctatcataagtaccacctctactcagagattcagtttgatatccccgaaaaaccagagttggtgactttcaacaccacatttggaaggtttggcattttcacgtgctttgatatcctcttccatgaccccgccgtgaccctagtgagagatttccacgtggacaccatactgtttcccacagcatggatgaatgttttgccccttttgacagctattgaattccattcagcatgggcaatgggcatgggagttaatcttcttgcagctaacctacatcatgtcaggctaaatatgacagggagtggtatctatgcaccacactctccaaaagtgtaccactatgacatggaaacagacttgggaaagatgctcttggcagaactcgattcccatccccggtcttctcctagctacccagtagcagtgaattggagtacctacgccgtggctcaagagccattcccgctacagagaaaagcttttggcggatttatttcccgggatgagttcaatttcacagaactttttgaagatgcaggaaatcttacagtctgccaaaaagaattatgttgtcacttaagctacagaatgttaggaaaagaagagcatgaagtgtatgttgtgggagctttcacgggacttcatggccgaaggagaagagagtactggcaggtatgcacaatgctgaagtgtaaatcggctagtctcaagacttgtggacaaccggtagaaaccgcttccacgagatttgatatgttttccctcagtggtacattcggaacagagtatgtttttcctgaagtgctcctttctaaagtgcatctgtcccctggaaatttcgaggtgctggaagatgggcgtttgacaaacaagaatgggacacctggaccgatcttaaccgtgtctctgtttgggaggtggtacacgaaggattccctttacagctcatgtatgaccaacaatcccatgttaatttacctgctcatttccacactattaatgatgtttaaaatattgtaa

SEQ ID NO: 84 - protein, 520 aa, Bos taurus
MITSSLQTSVAVFALVILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLSCLAKNNSIYVVANMGDKKPCNSHNTTCPSNGHYQYNTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 85 - protein, 520 aa, Bos javanicus
MITSSLQTSGAVFALIILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLSCLAKNNSIYVVANMGDKKPCNSRNTTCPSNGHYQYNTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAANLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYTKDAHSISGGTSNSATAYLMIPTLLMIITLRDAVMV

SEQ ID NO: 86 - protein, 520 aa, Moschus berezovskii
MITSSLQTSVAVFALIILHVSTLDTFKAAVYEHAVILPNDTKTPVSPDEALFLMNKNIDILEKAIKQAAEQGAQIIVTPEDALYGWKFTRETIFPYLENIPDPQVNWIPCQDPHRFGQTPVQARLSCLAKNNSIYVVANMGDKKPCSSRNTTCPSNGHYQYNTNVVYDAKGKLVARYHKYHLYSELQFDVPEKPELVTFNTTFGKFGIFTCFDILFHDPAVTLVKDFHVDTILFPTAWMNVLPLLTAIEFHSAWAMGMRVNLLAANTHNVSLKMTGSGIYAPHSPKVYHHDMETESGKILFAEVDSHPRNSSTYPPAVNWSAYATSITPFPGAKNSFRGFISRDEFNFTKLSESAGNLTVCQKEICCHLSYRMLGKEGNEVYVLGAFAGLHGRRRREYWQVCTMLKCRSTNLTTCGQPVETALTRFEMFSLSGTFGTEYVFPEVLLTEIKLAPEKFEVLEDGRLVNKNGSSEPVLTVSLFGRWYTKDTHSISGGTSNSATMHLMIPTLLMIITLQYTVMV

SEQ ID NO: 87 - protein, 538 aa, Orcinus orca
MIISSFQTSVAVFALIILHVSALATFKAAVYEHAVILPNDTETPVSQDYALLLMNRNIDILERAIKQAAEQGAQIIVTPEDAIYGWKFTREAIFPYLEDIPDPLVNWIPCQDPHRFGHTPVQARLSCLAKDNSIYVVANIGDKKPCHSRDSTCPPNGHYQYNTNVVYDANGKLVARYHKYNLYSELQFDVPEKPELVTFNTSFGKFGIFTCFDILFHDPAVTLVKDFHVDTVLFPTAWMNVLPFLTAIEFHSAWAMGMRVNLLVANTHNVCLKMTGSGIYAPHSPKAYHYDMETELGKILFAEVDSYPRNSPTYPPAVNWSAYATTIKPFPGQKNSFRGFISRDEFNFTELFENAGNLTVCQKELCCHLSYRMLGKENEVYVLGAFAGLHGRRRREYWQVCTMVKCKSTNLATCGQPVDTALTRFEMFSLSGTFRTEYVFPEVLLTKINLAPGKFEVLKDGRLVNKNGSSEPILTVSLFGRWYTKDVRSISGSANRGRWRETEDWTRSGETSVCSPISVVPSSVPYLTAVVCSSSTWF

SEQ ID NO: 88 - protein, 520 aa, Mesoplodon densirostris
MIISSFQTSVAVFALIILHVSALDTFKAAVYEHAVILPNHTETHVSQDDALHLMNKNIDILERAIKQAAEQGAQIIVTPEDAIYGWKFTRETIFPYLEDIPDPLVNWIPCQDPHRFGHTPVQRRLSCLAKNNSIYVLANIGDKKPCNSYDSACPPNGHYQYNTNVVYDAKGKLVARYHKYHLYSELQFDVPEKPELVTFNTSFGKLGIFTCFDILFHDPAVTLVKDFHVDTILFPTAWMNVLPFLTAIEFHSAWAMGMRVNLLVANTHNVSLKMTGSGIYAPHSPKAYHYDMETELGKILFAEVDSYPRNSPTYPPAVNWSAYATTVKPFPGQKNSFRGFISRDEFNFTELFENAGNLTVCRNELCCHLSYRMLGKEENEVYVLGAFAGLHGRRRREYWQVCTMVKCKSTNLTTCGQPVDTALTRYEMFSLSGTFRTEYVFPEVLLTKINLAPGKFEVLKDGRLVNKNGSSEPILTVSLFGRWYTKDVRSISGGTSNSATTYLIIYTLLMIIALQNTVMV

SEQ ID NO: 89 - protein, 519 aa, Globicephala melas
MIISSFQTSVAVFALIILHVSALATFKAAVYEHAVILPNDTETPVSQDYALLLMNRNIDILERAIKQAAEQGAQIIVTPEDAIYGWKFTSEAIFPYLEDIPDPLVNWIPCQDPHRFGHTPVQARLSCLAKDNSIYVVANIGDKKPCHSRDSACPPNGHYQYNTNVVYDANGKLVARYHKYNLYSELQFDVPEKPELVTFNTSFGKFGIFTCFDILFHDPAVTLVKDFHVDTILFPTAWMNVLPFLTAIEFHSAWAMGMRVNLLVANTHNVRLKMTGSGIYAPHSPKAYHYDMETELGKILFAEVDSYPRNSPTYPPAVNWSAYATTIKPFPGQKNSFRGFISWDEFNFTELFENAGNLTVCQKELCCHLSYRILGKENEVYVLGAFAGLHGRRRREYWQVCTMVKCKSTNLATCGQPVDTALTRFEMFSLSGTFRTEYVFPEVLLTKINLAPGKFEVLKDGRLVNKNGSSEPILTVSLFGRWYTKDVRSISGGTSNSATTYLIISTLLMIIALQNIVMV

SEQ ID NO: 90 - protein, 520 aa, Sus scrofa
MIASSFQTTLAVFALIILHVRALDTFKAAVYEHAVILPNDTKTPVSWDDALLLMNKNMDILERAIKQAAEQGSQIIVTPEDALYGWKFTRETIFPYLEDVPDPQKNWIPCQDPRRFGHTPVQVRLSCLAKNNSIYVLANIGDKKPCNSHDSKCPPNGYYQYNTNVVYDSEGKLVARYHKYHLYHELQFDVPEKPEVVTFNTPFGKFGIFTCFDILFRDPAVTLVQDFHVDTILFPAAWMNVLPLLTAIEFHSAWAMGMRVNLLAANVHHVRLNMTGSGIYAPHSPKVYHYDMETDSGKILLAEVDAHPRTSPTYPTAVNWGAYTLSIQPFPILQNSFRGFISRDEFHFTELSESAGNLTVCHKELCCHLSYRMLAKEENEVYVLGAFTGLHGRRRXEYWQVCTMLKCKSTNLTTCGQPVETALTRFELFSLSGTFGTEYVFPEVLLTKINLAPGKFEVLKDGRLVNKNGTSEPVLGVSLFGRWYIRDVPLISGGPAPQQKMYLIISALLMIIALQNIVMV

SEQ ID NO: 91 - protein, 504 aa, Phyllostomus discolor
MPFHFQEGGQTRTSFRLWRSQRLLLTILSSSVSLSCRHHVADVHLLCPAWQDPGVGAQGAEIIVTPEDALYGWSFTRETIFPYLEDIPDPRVNWIPCQDPHKFGHTPVQVRLSCLAKDNSIYVLANIGDKKPCKSNDPTCPSNGYYQYNTNVVYDKKGQLVARYHKYHLYSEPQFDVPEKPELVTFNTNFGKFGIFTCFDILFHDPAVKLVKDFRVDTILLPTAWMNVLPLLTAIEFHSAWAMGMRVNLLVANTHHVRLNMTGSGIYAPHSPKAYHYDMDTELGKILFSKVDSHPRSSPDFPAAVNWADYATTIKPFPVQKGTFKGFISRDAFNFTELSASAGDLTVCQKDLCCHLSYEMSRKEENEVYALGAFSGLHGRRGREYWQVCTMLKCRTTDVTTCGQPVETALTRFNVFSLSGTFGTEYVFPEVLLTKIRLSPGKFEVLKDGRLVNKNGMSEPILTVSLFGRWYAKDAHSASGGIGNLETTNLLISTLLMITILQIL

SEQ ID NO: 92 - protein, 520 aa, Homo sapiens
MVTSSFPISVAVFALITLQVGTQDSFIAAVYEHAVILPNKTETPVSQEDALNLMNENIDILETAIKQAAEQGARIIVTPEDALYGWKFTRETVFPYLEDIPDPQVNWIPCQDPHRFGHTPVQARLSCLAKDNSIYVLANLGDKKPCNSRDSTCPPNGYFQYNTNVVYNTEGKLVARYHKYHLYSEPQFNVPEKPELVTFNTAFGRFGIFTCFDIFFYDPGVTLVKDFHVDTILFPTAWMNVLPLLTAIEFHSAWAMGMGVNLLVANTHHVSLNMTGSGIYAPNGPKVYHYDMKTELGKLLLSEVDSHPLSSLAYPTAVNWNAYATTIKPFPVQKNTFRGFISRDGFNFTELFENAGNLTVCQKELCCHLSYRMLQKEENEVYVLGAFTGLHGRRRREYWQVCTMLKCKTTNLTTCGRPVETASTRFEMFSLSGTFGTEYVFPEVLLTEIHLSPGKFEVLKDGRLVNKNGSSGPILTVSLFGRWYTKDSLYSSCGTSNSAITYLLIFILLMIIALQNIVML

SEQ ID NO: 93 - protein, 520 aa, Ochotona princeps
MITSSFLTCVAIFALITLHVGALDTFTAAVYEHAVILPKETETPISQDDALLLMNKNMNVLESAVKEAAKMNAQIIVTPEDALYGWRFTREIIFPYLEEIPDPQVNWIPCQDPHRFGHTPVQTRLSCLAKNNSIYVLANIGDKKPCNSQSSTCPRNGYYQYNTNVVYDSEGKLVARYHKYHLYSEPQFDVLEKPELITFNTTFGRFSIFTCFDILSHDPAVTLVKEFHVDTVLFPSAWMNVLPPLTAVGFHSAWAMGMRVNLLVANIHHVSQNMTGSGIYTPDSPKAYYYDTKRESGHLLVSELDSHPRNSPSYPTAVNWSAYAVTIAPFPVQKNTFIGFISQDGFNFTELLENDGNITVCHKELCCHLSYRMLEKEKDXVYVLGAFTGLHGRRRREYCQVCTMLKCQTTNLTTCGKPVETASTRFEMFSLSGTFAAEYVFPEVLYSEIRLSPGKFEVLKDGRLVNKNGVSEPILTGSLFGRWYAKDLPHKSCGNNNSTIFYMPVFTILMIAYLQNIVMV

SEQ ID NO: 94 - protein, 497 aa, Alligator mississippiensis
MMSLQPYLYAMISTLSVLQVFASDTYTAAVYEHAVILPDVTDKPVSLDEALVLMNRNIDVLEVAIKEAAQQGAHIIVTPEDSVYGWNFTREALYPYLEDIPDPQVNWIPCTDPQRFAPAPVQERLSCLARNNSIYVVANMGDKKPCNCSDPKCPRDGHYQYNTNVVFDSQGKLVARYHKYNLFRSETQFDYPKEPEMVTFNTSFGNFGIFTCFDILFHDPAVVLVKKLHVDTVLFPTAWMNVLPFLSAIEIHSAWAMGIGVNLLASNTHNTLMAMTGSGIYAPDGPRAYHYDMETENGHLLTTQLSSHPHLSPTYPAAVNWSSYATSIKQFHVDKNTFMGAVRSDNFTFTELITIAGNHTVCQKDLCCHLIYRMVEKREDEVYVLGAFDGLHGSVIKYYWQICTLLKCKTMDLKTCGQPVTTTQTRFEAFSLSGTFGTKYVFPEVLLSGVRLAPGEFQVLNDGCLKNNNGPAKLILTVTLFGRYYEKDPPHPLRNSP

SEQ ID NO: 95 - protein, 497 aa, Ceuthmochares aereus
MLPSQNLLHTAVLALTVLQALASDTFIAAVYEHSVILPNATDKPVSPNDALMLMNKNMDVLERAIKEAALQGAHIIVTPEDGIYGWRFTRETIYPYLEDIPDPAVNWIPCINPTRFSPAPVQERLSCIARNNSIYVVANTGDKKPCNSSNHDCPSDGRYQYNTDVVFDPEGKLVARYHKYNLFRGETQFNYPKEPEIVTVETPFGKFGIITCFDILFREPATVLVSKLQVDTVLFPTAWMNVLPFLTAVEFHSAWAMGMGVNLLSANTHNINASMTGDGLFTPEGPAAYRYDSKTEEGRLLLAELSSRPRLSPTYPPSVNWSLYATSIKKFPEEKDTFAKAVQRDIFTFSELGYTAGNRTVCQGDLCCHLVYQMSGKSKDEVYVLGAFDGLGGSLIKYHWQICTLLKCKSTDLNTCGQPVEMAQTKFEMFSLSGTFGTNYVFPEVLYSEVQLAPGEFEVLHDGRLKSKHGTSKPLLTATLFGRLYEKDPPHPLRTSV

SEQ ID NO: 96 - protein, 518 aa, Felis catus
MITPRLLAYVAISVSCVLKVSSLDTFIAAVYEHAVILPNTTLIPVSREEALALMNRNLDLLEGAITSAAKQGAHIIVTPEDGIYGWNFNRETIYPYLEDIPDPRVNWIPCNYPNRFGHTPVQERLSCLAKHNSIYIVANIGDKKPCNASDPQCPPDGRYQYNTDVVFDSQGKLVARYHKYNLFMGEDQFNVPKKPEVVTFDTIFGRFGIFTCFDILFHDPAVTLVKDFHVDTIVFPTAWMNVLPHLSAIEFHSAWAVGMGVNFLAANIHHPHKRMTGSGIYAPDSPRAFHYDMKTKEGKLLISRLNSSSYDPGVVNWASYASSIKAFSTGNQEFKSTAFFDEFTFLELTRVTGNYTVCQKELCCHLSYKMSEKKPDEVYALGAFDGLHIAEGSYYLQICTLLKCKTPNLRTCGDAVESASTRFEMFSLSGTFGTQYVFPEVLLSDSQLAPGEFQVSSDGRLFSVKPPSGPVLTVTLFGRLYEKDWILKASSDLRAQAPGVMLVVIALSVCSFSWSPIG

SEQ ID NO: 97 - protein, 513 aa, Phodopus roborovskii
MSVSRWLACAVLSVFYVLQVSSLDTFVAAVYEHAVIMPSDTPSPLSHSEALAIMNRNLDLMEGAIVSAAKQGAHIIVTPEDGIYGVHFTRDTIYPYLEDIPDPQVNWIPCDNANRFGYTPVQERLSCLAKNNSIYVVANMGDKKPCNTSDPQCPPDGRFQYNTNVVFDSLGKLVARYHKQNLFMGEEQFNAPTEPELVTFNTPFGRFGIFTCFDILFHDPAVTLVTKFQVDTILFPTAWMDVLPHLAAIEFHSAWAMGMGVNFLAANIHSPLRRMTGSGIYAPDSPRAFHYDRKTNEGKLLLAQLDSHPSSSSVNWTSYASSVETPSTGDQEFQSVVFFDEFTFVELKGITGNYTVCQNDLCCHLSYQMSEKRADEVYALGAFDGLHTVEGQYHLQICTLLKCKTTNLRTCGSSVDTAATRFEMFSLSGTFGTQYVFPEVLLSQVKLAPGEFQVSSDGRLFSLKPTSGPVLTVTLFGRLYEKDWVSSTSSDLLTHSLMIIIMIIITPIIHYLC

SEQ ID NO: 98 - protein, 553 aa, Moschus berezovskii
MIMSQLLNYVAFLFFCVSRADSLDTFIAAVYEHPVILPNATLVPVSPEEALAVMNRNLDLLEGAVTSASKQGAHIIVTPEDGIYGFNFTRESIYPYLEDIPDPQENWIPCNNPNRFGHTPVQQRLSCLAKDNSIYIVANIGDKKSCNASDPQCPPDGRYQYNTDVVFDSKGKLVARYHKQNLFLSEDQFNAPKEPEVVTFNTTFGKFGIFTCFDILFHDPAVTLVRDSHVDTILFPTAWMNVLPHLSAIEFHSAWAMGMRVNFLAANLHYPLKKMTGSGIYAPDSPRAFHYDMKTEEGKILLAQLDSHPHPTPVVNWTSYASGVEVHSMGNQEFTDTIFFDEFTFLELKGIGGNYTVCQRDLCCHLSYKMSEKRADEVYALGAFDGLHTVEGSYYLQICTLLKCKTTDLHTCGDSVETASTRFEMFSLSGTFGTQYVFPEVLLSEIQLAPGEFQVSSDGRLFSLKPTSGPVLTVTLFGRLYEKDSAPNALSDLTTQALRIRRLVSIHNYRSECCIIPWRRKWQPTPVFLPGEFPWTEEPGGLQYMGSRRVGHD

SEQ ID NO: 99 - protein, 491 aa, Amazona guildingii
MLPSQPLLHTAVFALTVLQALASNTFIAAVYEHAVILPKAAIKPVSPDDALALMNSNMDILEGAIKEAAQQGAHIIVTPEDGIYGWRFTRETIYPYLEDIPDPVVNWIPCTDPTSIYLELKEVPFRNPAAYTVCCITQGWSEWCQQRTSRGLKFVHSPPLPXVVYDPEGKLVARYHKYNLFKGETQFNYPKEPEATTFETPFGKFGIFTCFDILFHEPAVVLVSELQVDTVLFPTAWMNVLPFLTAVEFHSAWAIGMGVNLLSANTHNINFAMTGSGLFTPEGPVAYHYDSETDEGRLLVAELSAQPRLSPNYPPAINWSLYATSIKKFPEEDTFLGAVRKDIFTFSELRHKAGNYTVCQRDLCCHLIYQMSNKSKDEVYVLGAFNGLHGSFIKYYWQICTLLKCKSTDPETCGHPVETAQTKFEMFSLSGTFGTNYVFPEVLYSGVQLAPGEFEVLSDGRLKSKHGTSKPLITATLFGRLYEKDLPHPLR

SEQ ID NO: 100 - protein, 520 aa, Patella sp.
MKKNLDIYAEQTRIASSQNVDIIVFPEDGIYGLNFNRDTIKPYLEDIPDPTEITWNPCNQSEYTNGVEVQITLSCMARNNGIYIVANMGDKKVCDADSICPDDGQRQYNTDVVFDPQGNLVARYHKQHLYFEAQFDTPSTVERVTFDTPFGKFGIITCFDILFKSPAVDLVEDLGVKNVIFPTAWMDSLPLLSAIQFHSAFAKLFKVNLLAANLHFPLFNFHGSGIYSPRRTEAYYYNDGLFSPGKLLIADVKSSPTHYRRHQVVPDTHGFRGGNSLGASFVHGQDTAEVNTQSRRNGGHYNTASRNGIISHQLADQASGVHLNNDLGPLTHRSIRNRIHNHAHLHRVNRHMVENGWATIMADNDNYQYLPLTEPKAKVKICQNEVCCYLDYQKQNTSNDELYALGVFDGLHQIEGLSWYTQVCVLLKCEFTDIASCGQPTKIAAGHFEYLKLWGTFTTEYVFPEVLVSDDGKLRLEHEHWKYLHNRIEFVNRGKESPLLSATLYGRHYDRDNSTVSNSV

SEQ ID NO: 101 - protein, 657 aa, Dreissena polymorpha
MFLELRLNLSIDRKNNIMEQNCLTKYFKTLLVCSLFCLTQCLKTDIKKFFTAAVYEHAVYQPPVTPLPLPRAQALEIMMTNIRVYQEQTKIATSKGAHIIVFPEYGVYGLSYKTRELIFPFMENIPEPERNWNPCEHPDVYPETEIQFALSCMARENNIYVVANIGDKQPCNVSLDPSCPRTGYYQYNTNVAYDAMGTLVAKYHKYNLFYENWFDKPSKVDISVFNTPFGRFGLIICFDILFEHPPIDLICRNNISNIAFPTAWMDAHPFYTSIQFHSAFAVGLGVNFLAANIHYPSLRFHGSGIYTQQGAAGYCFNTSNESGGQLIVQKIPVVDQSRLDACRGLNISLSDLNDQMLELDKTVNNGRETLQHTKRKGWGKGREKGRGNGQGQGKMHGHGQRNGQGQGKRKETRQEKGRGKRKGQGNGQGKGKGPRKLDLLSQPKFHKDMFPYDLYKNPFNLVPLRPEGRKTIVCHTDLCCHLSFSFEPTDRRSAEKPTRKSHAPDDFQASQHSTDLRNFTENRKDQDDIRAHLADNQRSSVEDFYAIGAYDGRYKNRSTGYYIQVCIVLRCRKQQNVVNCSASMAEDFRIRLSHFRLVGNFSATFQYPSILLQGESDFRLTEHSRRWSFSNGQLVANERFSSPLAVATIFARDYGRD

SEQ ID NO: 102 - protein, 533 aa, Patella vulgata
MLPVILVVAGILLNSMSTNAYNRKTFTAAVYEHSLIFPPNRSTVASRHNALADVMVNLRIYDQQAKFAASQDVDILVFPEDGLYGYTFNRDSIKHYLEEIPDPKSEVWNPCTQPNRYSKTEIQVYLSCMALNNSMYIVANMGNKQRCTRRTDSNCPSDGQYQYNTNVVYAPNGRLIARYHKQNLFYEYQFDRPRSVNYAFFETPFGRFGTFTCFDILFEEPAITLIERYNVSNIVFPTAWMDALPLLSAIQFHSAFAQGMGVNFLAANIHVPNKRFHGSGIYTPDGPKKFYYNDVGYGGGKLIISQLNVIENRHQHVAYNKVQRPSSEQKTFHSYVFYDKFTFVELQRSSGVLSVCNNRLCCHLEYAINKDNSDMYAFGAFDGLHTHEGQYYLQICTLLRCANKTRSSCGQPTKQAYSEFNDIKIYGTFQTNYVFPEVLKSRKRDGQLNLATNGWKYNNGVLRATRKTTHPVLSVSLFGRIYSRDKLPPGYHFTGSGSKSLSSSLYVSLLTPALLLAVYGSSIACGLFTRMNN

SEQ ID NO: 103 - protein, 517 aa, Mizuhopecten yessoensis
MIIKFIYVAVLTSSVGGFLFQNETRTTFRAAVYEHAVHLFPSSSSTTRQTALANMMMNLEEYKRQADIAGGQDVDILVFPEMGIYGLGLSREGIAPYLENIIDPEKEVWSPCDYPHRYPNSEVQHFLSCLAKNNSLYIVANFGDRQPCSQSLDPNCQAQYQYNTDLVYDPRGILVAKYHKVNLYFEKSFDFPPVNKAVTFDTPFGVFALAICFDVLFYDPIIDLIINHGVRNVAFPTAWMDRLPFLSSIEYHSAFAAGMGVNMLAANINYPRMRFHGSGIYTPNGASRFIYNDQTASGKLLVSDLSVINKHGSVTLPPRTPTLKTPHVTGSIFKSKLHYDSYNLTLISGISGHATVCHNTICCQLNYSRQNTNETFAFGAFDGLHGRNGSYFMYLQVCTLLKCTRTGNTSCDDRNMQSQTMFNNVHITGYNFTSYIYPEVLLSQNGSLGLSIGRWTYSYGVLHAPTGYEAPLVSTSLYGRLYSTDDSNGRESCSIRLINRKSLITDLVVTLTCRISE

SEQ ID NO: 104 - protein, 520 aa, Octopus sp.
MNFVYTIIFFLQLPVLLSAAEKTFTAAVFEPTIPRRSSMGLVSRDNAIEQMKHVLKGYAEVAARASKKGVDILVFPENGLIAHLDGVTRLGIRNFLDLIPEPNKEKPWIPCDEPYLYPRTEIQQVISCIAKKNKIYLVVNVGDIQPCNRTDDANCPYDGRYQYNTNVVYSSEGALVSRYHKKHLFYEPYYNSPKIAEYGVFDTPFGRFASIVCFDIYFNDSLNELVMKHQVSNILFPNLWFDTPPFFFSSLFHNSIAKAFNINLISANIKINNFVRGGSGIYTPSINRYVYNPLNPAPSDEYIQLQVKNLEKSKRTFPCPPIRKVPESDNPDVKTATMTNLFPVSAVMLHKYSGVVSVCKYNACCSLQYEFMSNRTSVFNINYILAITNEESVDLTNPEGVDMQSCILCPIKDFKMSCLMSLPAGIPDGEVSFRKLKMTAHNFNTSYFFPHIATFNSSTYDINTSWKHDRGEIEITEPVKKVFNAVIFGRLYPNSATATFAIFPAILLILLNLISKELLV

SEQ ID NO: 105 - protein, 554 aa, Formica exsecta
MRTRILDLLLIYSSLNFCCYGNLEDCTDGPDEDYCNENFISAPAQISTPDSPTYIAAVVEYHPEYTTDSNETLKKNSDAYVEHIKTARVHNADIIVFPEDGLTTVKFPKREEMQNWTTIIPSASFNYTPCTDSTIEVSETLKKISCAARNNTIYVVINIAEKLPCIEDVCPKDKMFYYNSNVVFDRTGKIIARYRKTNLFGEQQFNVTSKPEIVIFDTDFGVKFGTFICFDILFHEPALQLTRVHQVTDIVYPTAWFSEAPFLTAVQTQAGWSFSEDVNFLVSGYNEPASGNTGSGIYLGREGIGKAIMSKTTRDEILVFEVPKKKKVFNKDHHDHSNDYDHKVYNHKKENIHEELWRKQFSNTMADNDTIFLIHDNINVFETFSLEGNVTRNVCKNNFCCDFKVEVVTIDPSKKYRLIAFSGIRLYATVEAGVRACGIVQCLNDSVSSCGSVHDSKTVFSRIEIDTTFYDYKNILIMPSTLRSDLLPLSENWTYNEHTHDDHVHISMLLNNNTNNLTTFGIYSRYFNKNNASRIVFDIYYFIALLVSLCLSRL

SEQ ID NO: 106 - protein, 516 aa, Drosophila simulans
MAKNYWGFSLFCLALGLMLNLSQQASLFSRDFYTAGVVEFKPSNDLSDNLAGYLEIIHSQNATSTDIIVFPESTLNSAGSTTFVPNPEDQIIPCLSDPNATYYEEFLVTLSCAARNASKYIVINLTEKQKCEDVPEDTRPCASNGLNVFNTNVVFDRQGVVVSRYRKVHLYGEPKNSTFLPELSTFETDFGVTFGHFICFDILFYTPAHQLVDQGVTDFVYPAMWFSQLPFLTAVQIQLGWSYANNVNLLAAGASDPIVGNSGSGIYHGRSGSLRSVMRQESGERSIYVARVPKYRAKRRMKRDLNRQVATSSSFNIKRDYLENFTSEELKIDDGKTGNLSQNLCHGGFCCHFDLAWRSLRKPARNTSHYSYRVGIYEGWRNEKRLDVNYIRNCGLFTCSGDSIDDCGQLLPDLHRPLVTFTHLEIRVTYPQSRKFLLFPDTLLDNLLPLEPKQFEWSQKRISEESQQVRLALRKSLEVKHLLTFGIYGNYYNNECTYGVGTTEEQLECGYKNPKI

SEQ ID NO: 107 - protein, 535 aa, Trichonephila clavipes
MKRILDFQLFFTTVLSLLALELAAGPDHYRAAVAETVRFSDISCPPKEVVKRNLDIYEAAVKASAKNDVDIIVFPECGIFPCEKVNRSVLRTFVEDIPNPKRESINPCKQSDKFHNHSILSKLSCLAKKHNLYIVANTFDFKKCKPVETCDADGFDTCTSEESNCPSDGNFFYNTNIVFDRKGSLVSRYYKKHLYFERTLNKPDFQQDATFHTDFGNFTTVICFDLIFKESVEDVKKPGIINLAYPTYWYDHVPQVFFATAYQQAWAMTNKVNLLAANGHMPKTGTLGSGIYSAHKGALIYTHNPDFRTKLLISNVPRSLNKEKANSIELNEKKFYLESGAAIEVVGEEEEERNFRSDCTTTVLGEANDTMDSYRCFDSTLNDYQYFKLEGNEGTHEICSNEFCCSLSYQAHSMDEDYFLAVSGKPLTYYDTFSYGIQSCFLARCRSSDGHPCKHFLLKSRTIFQNVHIKANFTTRHVYPFALDSDVRLTNRKNWSFDGKSQITYENLNKKSSVTFFGLHGRLYDVDKILKKLLK

SEQ ID NO: 108 - protein, 509 aa, Parasteatoda tepidariorum
MTTSWFLLLSMFFVACTDIDATAYTAAAYDHARVGRLNDPPNLVVEMNLKKYIKAGKIAKSKGADIIVYPEYGIFYPGNRTQLKHFLEDVPDPKKTRTAPCDEVELYMKRPILYTLSCVAKHNAMYVVANMGDIKDCSGVSECPEDGVFHFNANIVFDRNGRLILRYYKEHLFFELGMDLAREEQDPVFTTDFGTFATYICFDILFKRMSQVAKWPQIDGIMFTSMWAENPPQGLSVAFWQGWALGNNATIVVSNIQSPSRYALGSGIFHKTHGSLGYTYNPDRVSKVVVAKVPVSHQPVLTSRPSLTAFTDTKVWDMKSEEEDVPKVCSNLLIDATDDSTEKLYCSDEKTTNYTLVKLPKSGGHVKACNNGMCCSVSYSAETIEEDYYLGVYNGTINAYDLNRYCWCEENCFLARCDAIKGRPCSTFPRKTNTVFYHVELKSNFSTPFVYPSVVSSKSRLVPMTEWEFDVGHSSDSQSSIHFHSKYGEQLLSVGFRARCFDRDPPYVR

SEQ ID NO: 109 - protein, 487 aa, Rhipicephalus sanguineus
MQGMWLLLTAVLCSSTAHAFKAAVYEHVQSKATPTETAVAENLEAYLWATGNASSEKADIIVFPEDGILYRLKDREEVAKWAEDIPDLGVMACGSQNMSLLSNLSCMAQSNSIYLVANLIDRKPCANETLTTAVTATRSSISTTNGPSDRNGPLSQVILHMLLIETFSISRCILPTTRYHKNHLFVEPLMNPADPYEFAVFKTDFGAQVGLFICFDVLFAESSLLVEKHNVTLGVMSSWWFDELPGWYSVGVQQAWSIHNGIPLLASGIQRLEMGSLGSGIYAGLRGPLNYTYSPDGKSKLLMADLSNATSLNPRYPGDELHPKQRFLNQGDVSDHAAQVLGRPKGYSRVCHGDFCCSLSYETQELNDNFVLLARNGLRNIGTYMQLGIQQCILAVCESRNNTVCKNFSTRSTTKFTELQLTAQFATNAVFPVVASDELALTPKDKWQFETTSTNASTLTLKENGKDEDILHIVLYARLYDADRFTK

SEQ ID NO: 110 - protein, 524 aa, Caerostris extrusa
MMASGFKLLCLAVALHMSSIGLCADEKYYRAAVHEIRQYNDNSSSGSELISKNMELYNQAADIASFENCDILVYAEKGLFPSKKSNSSWFIYYAEDVPSGKERANPCEQEEYSDRPILKNLSCIAQEHSLTVVATLIDIKKCIVSEDCERTTINYCVSNADDCPEDGIYRFNTQVVFNKRGTLIARYYKQHPFFEEGITTPKYAANVYYKTNYGTFVSYIGFDFFFYDSVKGVEKPWINAVTYANWWFDHTPFNYFSVSNQQAWSMNNGVAVLSSDVHEPNLGSLGSGIYIPGKGALIYSYNPDGKSKLLISNVPKSNSLSDNESLNTKFYYIDENGTGTVLNGEEPRNFKSQCSIDVLGEETSSFTDYRCHKTEVHQYTFTKLNDTSGQIDTCSNGFCCSLSYTAESMNETFYFGVSGHRLKFYDTYWFGVQACFLARCEDVDGKQCRNFLLTSDTIFHDVEITGSFETKHIYPQAMDTELRLTDRNEWKFDGQSMSYESVNQNPLLFMGLYGRLYRRDGLVK

SEQ ID NO: 111 - DNA, 1503 bp, Mus musculus
  source 1..1503: mol_type = genomic DNA
atggcttcattacattttcctcaatgggcagtgagttttgtcttctttgcccaggctgtgggttcaatggacacttttattgctgctgtgtatgaacatgctgttatactgccaaacaaaactgaaagtcctgtttccactgaagaggctttgctcctgataaacaagaacatagacattttggagagtgcaatcaagctggcagccagacagggtgcacatatcattgtgacgccagaagatggaatctatggttggatcttcaccagggagaccatttacccctacctagaggatataccagaccctgaagtgaactggattccctgtagagaccctaggaggtttggctacacaccagtacaggagagactgagctgccttgccaaggagaactctatctatattatggcaaatattggggacaagaagccatgcaatgctactgatcctcattgtcccccggatggccgttaccaatataataccaatgtggtcttcgattctaagggtaggctaacagcccgctaccataagtacaatctttttgaaccagagattcagtttgatttccccaaagattcagagctggtgacctttgacaccccgtttgggaagtttggcatcttcacttgctttgacattttctcttatgacccagctgtggtggttgtgaaggacacccaggtcgacagtgttctcttacccacggcgtggtacaacaccctgcccctgctttcagcagttccattccattcggtgtgggccagagccatgggggtcaacgtgcttgctgcaaacacccacaacaccagcatgcatatgacagggagtggaatctacagcccggaagctgtccgagtgtaccactatgacatggagacagagagtggccaactgctgctttcagagctgaggtctcggcctcgccagcacgccacccctgcagaggttaactggagcgcttatgccaggactgtgaagccgttctcatcggggcaggcagacttcccaggaaagatttattttgacgaatttagcttcaccaagcttacaggaagtgctggcaattacacagtttgccaaaaggacctgtgctgtcacctgacttacaagatgtctgaaagccgaatggacgaggtgtatgttctgggtgcctttgatggactccatacaggggaaggccagtattacctacagatatgtacattgctgaagtgtcaaaccaccaactcgagaacttgtggggaacccgtggggtcagcttttacaaagtttgaagaattctctctcagtggcacctttcggacaaaatatgttttcccacagatcgtgctaagtgggagtcaacttgccctggaaagatattatgaagtctcaagagatggacgtctgaggagtcgaggtggagcccctttgcctatcttagtgatggccctgtatggaagagtgtttgagagagaccctccgcgcttagggcagggacctgggaagctgcagtga

SEQ ID NO: 112 - DNA, 1503 bp, Mus caroli
  source 1..1503: mol_type = genomic DNA
atggcttcattacattttcctcaatgggcagtgagttttgtcctctttgcccaggctgtggggtcaatggacacttttattgctgctgtgtatgaacatgctgttatactgccaaacaaaactgaaagtcttgtttccactgaagaggctttgctcctgataaacaagaacatagacattttggagagtgcaatcaagctggcagccagacagggtgcacatatcattgtgacgccagaagacggaatctatggttggatcttcaccagggagaccatttacccctacctagaggatataccagaccctgaagtgaactggattccctgtagagaccctaggaggtttggctacacaccagtacaggagagactgagctgccttgccaaggagaactctatctatattgtggcaaatattggggacaagaagccatgcaatgctactgatcctcattgtcccccggatggccgttaccaatataataccaatgtggtcttcgattctaagggtaggctaacagcccgctaccataagtacaatctttttgaaccagagattcagtttgatttccccaaagattcagagctggtgacctttgacaccccgtttgggaagtttggcgtcttcacttgctttgacattttctcttatgacccagctgtggtggttgtgaaggacgcccaggtcgacagtgttctcttacccacggcgtggtacaacaccctgcccctgctttcagcagttccattccattcagcgtgggccagagccatgggggtcaacgtgcttgctgcaaacacccacaacaccggaatgcacatgacagggagtggaatctacagcccagaagccgtccgagtgtaccactacgacatggagacagagagtggccaactgctgctttcagagctgaggtctcggccccgccagcatgctacccctgcagaggttaactggagcgcttatgccaggactgtgaagccgttctcatcggggcaggcagacttcccaggaaagatttattttgatgaattcagcttcaccaagcttacgggaagtgctggcaattacacagtttgccaaaaagacctgtgctgtcatctgacttacaagatgtctgagagccgaatggacgaggtgtatgtcctgggtgcctttgacggactccatacagtggaaggccagtattacctacagatatgtacactgctgaagtgtcaaaccaccaactctagaacttgtggggaacccgtggggtcagcttttacaaagtttgaagaattctctctcagtggcacctttcggacaaaatatgttttcccacagattgtcctaagtgggagtcaacttgccctggaaagatattatgaggtctcaagagacggacgtctgaggagtcgaggtggagcccctttgcctatcttagtgatggccctgtatggaagagtgtttgagagagaccctccacgcttagggcagggacctgggaagctgcagtga

SEQ ID NO: 113 - DNA, 1503 bp, Mus pahari
  source 1..1503: mol_type = genomic DNA
atggtctcattacattttcctcaatgggcagtgagttttgtcctctttgcccagactgtgggttcaatggacacttttattgctgccgtatatgaacatgctgttatactgccaaacgaaacagaaactcctgttcccaccgaagaggctctgctcctgataaacaagaacatagacattttggagagcgcaatcaagctggcagccagacagggtgcacatatcattgtgacgccagaagacgggatctacggttgggtcttcaccagggagaccatttacccctacctagaggatataccagaccctgaagtgaactggattccctgtagagaccctaagaggtttggctacacaccagtacaggagagacttagctgcctcgccaaggagagctctatctatattgtggcaaatattggggacaagaagccctgcaatactactgatcctcactgtccccgggatggccgttaccagtataataccaatgtggtcttcgattctaagggtaggctaacagcccgctaccataagtacaatctttttgaaccagagattcagtttgatttccccaaagattcagagctggtgacctttgacaccccgtttgggaagtttggcatcttcacttgctttgacattttctcttatgacccagccgtggtggttgtaaaggacgcccaggttgacagtgttctcttacccacggcgtggtacaacaccctgcccctgctatcagcagctccattccattcagcgtgggccagagccatgggggtcaacgtgcttgctgcaaacacccacaacaccaggatgcacatgacagggagtggcatctacagcccggaagccgtccaagtgtaccactacgacatggagacagagagtggccaactgctgctttcagagctgaggtctcggccccgccagcatgccacccctgcagaggttgactggagcgcttatgccaggactgtgaagccgttctcatcggggcaggcagacttcccgggaatgatttattttgatgaattcagcttcaccaagcttatgggaagtgctggcaattacacagtttgccaaaaggacctgtgttgtcacctgacttataagatgtctgagagccgaatggacgaggtgtatgtcctcggtgcctttgatggactccatacagtggaaggccagtattacctacagatatgtacattgctgaagtgtcaaaccaccaacttgagaacttgtggggaacccgtggggtcagcttttacaaagtttgaagaattctctctcagtggcacctttcggacaaaatatgtcttcccacagatcgtcctaagtgggagtcaacttgccctggaaagatattatgaagtctcaagagacggtcgtctgaggagccgaggtggagccgctttgcctgtcttagtgatggccctctacggaagagtgtttgagagggaccctccacgcttagggcagggacctgggaagctgcaatga

SEQ ID NO: 114 - DNA, 1503 bp, Arvicanthis niloticus
  source 1..1503: mol_type = genomic DNA
atggtttcattatattttttacaatgtgtagtgagtcttgtcctctttgctcaggctgcgggttcaatggacacctttattgctgcggtatatgagcatgctgttatactgccaaacaaaacagaaagtcctgtttccaccgaagaggctttgctcctgatgaacaagaacatagacattctggagaatgcgatcaagctggcagccagacagggtgcacagatcattgtgaccccagaagatggaatctatggctggatcttcaccagggagactatttacccctacctagaggatataccagaccctgatgtgaactggattccctgtagagaccctaaaaggtttggctacacaccagtacaagagagactcagctgcctcgccaaggagaactctatttacatcgtggcaaatattggggacaagaagccatgcaacgctactgatcctcagtgccccagggatggccgttaccaatataataccaatgtggtcttcagttctgagggtaggctagtagcccgctaccataagtacaatctttttgaaccagagattcagtttgattttcccaaagattcagagttggtgacctttgacaccccctttgggaagtttggcgtcttcacttgctttgacattttttcttacgacccagctgtgatggttgtgaaagactttcaggttgacagtgttctcttacccacggcgtggtacaacactctgcccctgctttcagccgttccattccattcagcatgggccagaggcatgggggtcaacctgcttgctgcaaacacccacaacaccagaatgcacatgacagggagtgggatctacagtccggaggctgtcagggtttaccactacgacatggagacagagagcggccagctactgctttcagagctgaggtctcggccccgccagcaggcctcccctgcagacgttgactggagtgcctatgccaggactgtcaagccattctcatcggagcaggccgacttcccagggatgatttattttgatgaattcagcttcaccaagcttacaggaagtgctggcaattacacagtttgtcaaaaagacctgtgctgtcacctgacttacaagatgtctgagagccgaatggatgaggcttatgccctgggtgcctttgatggactccataccgtggaaggccagtattacttacagatatgtacattgctaaagtgtcaaaccactaacttgagaacttgtggggaacccgttgggtcagcttttacgaagtttgaggaattctctctcagcggcacctttgggacgaaatatgttttcccacagatcgtcttaagtgggagtcaactggccctggaaagatattatgaagtctcaagagatggacgtctgaggagccaaagtggggcctctttgcctgtcttagtgatggccctgtacggaagagtgtttgagaaagattctccacgcttaggtcagggacctgggaagttgcaatga

SEQ ID NO: 115 - DNA, 1506 bp, Homo sapiens
  source 1..1506: mol_type = genomic DNA
atgattatatcacattttccaaaatgtgtggcagtttttgccctccttgctctgagtgttggtgcactggacacttttattgctgcagtatatgagcatgcggtgatattaccaaacagaacagaaacacctgtttcaaaagaagaagctttgctcctgatgaacaagaacatagatgttttggagaaagcagttaagctggcagcgaagcagggtgcacatatcattgtgaccccagaagatggaatctatggttggatcttcaccagggagagcatttacccctatctagaggatataccagaccctggagtgaactggattccatgtagagacccctggagattcggcaacacaccagtgcaacaaagactcagctgcctggccaaggacaactctatctatgtcgtggctaatattggggacaagaagccatgcaatgctagtgactctcagtgtccccctgatggccgttaccaatacaacactgatgtggtgttcgattctcagggaaaactgttggcacgctaccataagtacaatctttttgcacctgaaattcagtttgatttccccaaggattcagaacttgtgacttttgacactccctttgggaagtttggcatttttacttgctttgacattttttctcatgacccagctgcggtggtggtggatgaagtttcaattgacagcattctctaccccacagcatggtacaacacgctgcccctcctctcggctgttcccttccattcagcatgggccaaggccatgggagtcaatctacttgctgcaaatacccacaacaccagcatgcacatgacagggagtggaatctacgccccagaagcagtcaaggtgtaccactatgacatggaaacagagagtggtcagctgttgctatcagaactgaagtctcggccccgccgtgagcccacctaccctgcagctgttgactggcatgcgtatgccagcagtgtcaagccattttcctctgaacagtcagattttctggggatgatttattttgatgagtttaccttcaccaagcttaagagaaatacaggaaattacacagcttgccagaaagatctgtgttgtcacttaacttacaagatgtctgagaagcgaacagacgagatctatgccctaggtgcttttgatggactgcacacagtagaaggccaatattacttacagatatgtgcattactgaagtgtcaaaccactgacctggaaacgtgtggagaacctgtggggtcagcttttaccaagtttgaagacttctccctcagtggcacatttggaacgcgttatgttttcccacagatcattctaagtgggagtcagcttgcccctgaaagacattatgagatttcaagagatggacgcttgaggagccgaagtggagcccctttgcctgtcttagttatggccctgtatggaagagtgtttgagaaggaccctccacgcttagggcagggatctgggaaattccagtga

SEQ ID NO: 116 - DNA, 1506 bp, Cynocephalus volans
  source 1..1506: mol_type = genomic DNA
atgattatatcacattttccaaaatgtgtggccatttctgccctctttgtcctgagtgttggtgcactggatacttttattgctgcagtgtatgagcatgctgttatcttaccaaacagaacagaaacacctgtttcaaaagaagaagctttattcctgatgaacaagaatatagatgttttggagaaagcagtcaagctggcagccaagcagggtgcacatatcattgtgacaccagaagatggaatctatggttgggtattcaccagggagagcatttacccttatctggaggatataccagactctgaagtgaactggattccatgtagagatcctcaaaggtttggctacacaccagtgcaagaaagactcagctgcctggccaaggacaactccatctatgttgtggcaaatattggggacaagaagccatgcaatgccagtgaccctcaatgtcctcccgatggtcgttaccaatacaacacggatgtggtgtttgattctgagggtaggctggtggcacgctaccataagtacaatctttttgcacctgaaattcaatttgatttccccaaaacatcagaagttgagacttttgacactccctttgggaagtttggaattttcacttgctttgacattttttctcatgacccagctgtggtggtggtggacaagtttcaagtcgacagtgttatctaccccacagcgtggtacaacacgctgccccttctctcggctgttccattccattctgcatgggccagagccatgggagtcaatctgcttgctgcaaatacccacaacaccagcatgcacatgacagggagtggtatctatgccccagaagcagtcaaggtatatcactatgacatggaaacagagagtggtcagctgatgctctcagaattgaagtctcggcctcgccgtgaacccacctatcctgcagttgttgactggggtgcatatgctgaaagtatgaagccattctcgtctgagcagtcacattttccagggctgatttattttgatgagttcaccttcaccgagcttaagagaaacacaggaaattacacagtgtgccagaaagacctgtgttgtcacttgacttacaagatgtctgagaagcgaacagatgaggtctatgcactaggtgcttttgatggactgcacacagtagaaggccaatattacttacaggtgtgcacattactgaagtgtcaaaccactgacctgagaacttgtgggcagccagtggggtcagtttttaccaagtttgaagaattctccctcagtggcacatttggaaccagttatgttttcccgcagctcattctaagtgggagtcagcttgcccctgaaagacattatgaggtttcaagagatggacgtctaaggggccgaagtggagcccctttgcctgtcttagttatggctctgtatggaagagtgtttgagaaggaccctccacgcttagggcagggacctgggaaattgcaatga

SEQ ID NO: 117 - DNA, 1506 bp, Sapajus apella
  source 1..1506: mol_type = genomic DNA
atgagtagatcatattttccaaaatgtgtggcactttctgccctccttgtcctgagtgttggtgcactggacacatttattgctgcagtatatgagcatgcggtgatattaccaaacagaacagaaacacctgtttcgaaagaagcagctttgctcctgatgaacaagaacatagatgttttggagaaagcagttaagctggcagccaagcagggtgcacatatcattgtcaccccagaagatggaatctatggttgggtcttcaccagggagagcatttacccctacctggaggatataccagaccctgaagtgaactggattccatgtagagaccctgggagattcggcaacacaccagtgcaagaaagactcagctgcctggccaaggacaactctatctatgtcgtggctaatattggggacaagaagccatgcaatgccagtgaccctcagtgtccccccgatggccgttaccaatacaacactgatgtggtgtttgattctcagggaaaattggtggcgcgctaccataagtacaatctttttgcacctgaaattcaatatgatttcccgaaggattcagaatttgtgacttttgacactccctttgggaagtttggcatttttacttgctttgacattttttcttatgacccagctgtggtggtggtggatgagtttcaagttgacagcattgtctaccccacagcatggtacaatacgctgcccctcctctcggctgttcccttccattcagcatgggccaaggccatgggagtcaatctgcttgccgcaaatacccacaacaccagcatgcacatgacagggagtggactttacgccccagacgcagtcaaggggtaccactacgacatggaaacagagagtggtcagctgatgctatcagaattgaagtctcggcctcgccgtgagcccacctaccctgtagctgttgactggcatgcttatgccagcagtgtcaagcctttctcctctgagcagtcagattttccagggatgatttattttgatgagtttaccttcactgagcttcaaagaaatacaggaaattacacagtttgccagaaagacctgtgctgtcacttaacttacaagatgtctgagaagcgaacagatgaagtctatgccctaggtgcttttgatggactgcacacggtagaaggccaatattacttacagatatgcacattactgaagtgtcaaaccaccgacctcgaaacatgtggagagcctgtgggttcagcttttaccaagtttgaagaattctccctcagtggcacattcagaacgcgttatgttttcccacagatcactctaagtgtaaatcagcttgcccctgaaagacattatgagatttcaagagatggacgcctgaggagccgaagaggagcccctttgcctgtcttagtcatggctctgtatggaagagtctttgagaaggaccctccacgcttagggcagggacctgggaagttacagtga

SEQ ID NO: 118 - DNA, 1506 bp, Panthera leo
  source 1..1506: mol_type = genomic DNA
atgattacatcatattttccaaaatatgtggcactatttgccatctgtgtcctatgtgtcggtgcactggacactttcattgctgcagtgtatgaacacgctgtcatattaccaaacagaacagagacaccagtttccaaagaagaagctttgctcctgatgaacaagaacatagatgttttggagaacgcagttaagctggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaagacaactccatctacgttgtggcaaatattggggacaagaagccatgcaatgccagcgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgttctctacccggtggcgtggtacaacacactgccccttctgtcggctgttcccttccactctgcgtgggccagagccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattctcgtctgaacagtcgaattttccggggatgatttattttgatgagctcaccttcattgagcttaagagaaacacaggaaattacacagtttgccagaaagacctgtgctgtcacttaacttacaagatgtcggagaaacgaacagacgaggtttacgcactaggtgcttttgatggactgcacacagtagaaggtcaatattacctacagatatgcacattactgaagtgtcaaatcagtgacctgcgaacttgtggggagcccgtggggtcagctttcaccaagttggaagaattctccctcagcggcacatttgcaacacgttatgttttcccacagatcattctcagtggaagtcagcttgctcctgaaaggtattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 119 - DNA, 1506 bp, Lynx canadensis
  source 1..1506: mol_type = genomic DNA
atgattacatcatattttccaaaatatgtggcgctatttgccatctgtgtcctatgtgtgggtgcattggacactttcgttgctgcagtgtatgagcacgctgtcatattgccaaacagaacagagacaccagtttccaaagaagaagctttgctcctgatgaacaagaacatagatgttttggagaacgcagttaaactggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagtgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgtcctctacccggtggcgtggtacaacacgctgccccttctgtcggctgttcccttccactctgcgtgggccagagccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgtagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgagaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 120 - DNA, 1506 bp, Panthera tigris
  source 1..1506: mol_type = genomic DNA
atgattacatcatattttccaagatatgtggcactatttgccatctgtgtcctatgtgtcagtgcactggacactttcattgcttcagtgtatgaacacgctgtcatattaccaaacagaacagagacaccagtttccagagaagaagctttgctcctgatgaacaagaacatagatgttttggagaacgcagttaagctggcagccaggcagggtgcacatatcattgttaccccagaagatggaatttatggctgggttttcacaagagagaccatttacccctatctggaagatatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagcgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgttctctacccggtggcgtggtacaacacactgccccttctgtcggctgttcccttccactctgcgtgggccagagccatgagagtcaacttgcttgctgcgaatacccacaacaccagcatgcacatgacagggagtggaatctacacaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgcgaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaatga

SEQ ID NO: 121 - DNA, 1449 bp, Hippopotamus amphibius
  source 1..1449: mol_type = genomic DNA
atgattatatcatattttccaaaatatgcggccactttggccttctttgtcctgagtgccggtgctctggacactttcattgctgcagtgtatgagcacgctgttgtattaccaaacaggacagaaacacctgtgccgaaagaagcagctttgctcctgatgaacaagaatatagatgttttagagaaagcagttaaactggcagccaggcggggcgcgcatatcattgtgaccccagaagatggaatttatggctgggtcttcacaaggggcaccgtttatccctatctggaggatatcccagaccctgaagtgaactggattccatgtagagattcccagaggtttggccgcaccccagtgcaagaaaggctcagctatttggccagggacagctctatctacttcgtggcaaatattggggacaagaagccatgcaataccagtgaccctcagtgtcccccgnacggtcattaccaatacaacactgatgtggtgtttgattctgagggtaggctggtggcctgctaccacaaggacaatctttttgcacctgaaattcaatttgatttccccaaggattcagaatttgtgacttttgacacaccctttgggaagtttggcattttcacttgctttgacattttttctcatgagccagctatggtggtggtggaggagttacaggttgatagtgttctccaccccatggcctggtacaacacactgcccctgctctcggcggtccccttccattctgcgtgggcccgagccctgcgagtcaatctgctagccgccaatacccacaccaccagcatgcacgtgacaggtacttcatgccgctcctgccccaatgggcagccgtgggtgtgctatgacacggaaacagagagtggccagctgatgctctcagaattgaagttgtggcctcgaagagaacgcagttaccctgcagctgttgactggagtgcttatgccagaggtgtcaaaccattcccatctaaactgttggattttccagggatgctttattttgatgagttcaccttcaccaagcttgagagaagcacaagaaattacacagcttgtcagaaagacctgtgttgtcacnnaacttacaagatgtctgagaaacgaacccatgaggtatatgtactaggtccctttgatggactgcacacggtagaaggtcaatattacttacaggtatgcacattactgaagtgtcaaaccactgacctgagaacttgtggagagcctgtggggtcagcttttaccaaatttgaagatctctccctcaggggcacatttggaacgcgttatgttttcccacagatcattcttagtgggagtcagcttgcccctgaaagacattatgaggtttcaagagatggacgtctgcggagccaaggtggaggcccttttcctgtcttggtcctggccctgtag

SEQ ID NO: 122 - DNA, 1500 bp, Gracilinanus agilis
  source 1..1500: mol_type = genomic DNA
atggctactgcctggtttcccacttgtgtgatagttttggcgatctgtatcctcgatggcggtgcctcagacacatttaaggctgctgtatatgagcatgctgttatattgccaaataggacagaaatacctgtttctcagaaagatgccctgatgttgatgaataagaacttagatgttttggagaaggcaatcaaattggcggctaagcagggtgcagacattattgtgactcctgaagatggagtttatggctgggatttcacaagggaaaccatttacccctacttagaagatatcccagaccctcaagaaaactggattccatgtactgaagcccacagatttggccgtactccagtgcaaaaaagacttagctgcttggccagagagaattctatctacgtggtggcaaatattggagacaagaaagaatgtggtgctaatgaccccacatgcccaccagatggtcggtaccaatacaacactaatgtggtgtttgattcagatgggaaactggtggcccgctaccataagtacaatctttttgcaactgaagttcagttcaacttccctaaagattcaccatatgtgacctttgatacaccctttggaaagtttggcattttcacttgctttgacatattttcttatgaacctgctgcagctgtggtggataaattccaagtcgacagcatactgtaccccacagcctggtacaatactctgcccctcttgtctgctgttccattccattcagcctgggccagggccatgagagtcaatctcctttcagcgaacactcacaacaccaggatgcacatgacagggagtggaatctatgcaccagaggcagttaaagtttatcactatgacatggaaacaaaaagtggccaactattgctctcagaactgaagacacggccacgactttcttctgactaccctgcagtggtagattggagttcctatgccaaaagtgccaagccattcatttctgatcagccaaagttccatggaagtatctattttgatgaatttaccttcattgaacttactaaagaaacaggaaattacaccatttgccagaaagaactctgctgtcacttgagctacaagatgtctgagaaaagaactgatgaagtctatgcactaggggcttttgatggccttcacattgttgaaggacaatattatttacagatatgcacattattgaagtgccaaacaactgacttaaaaacctgtggtgagccagttggatcagctttcaccagatttgaagaattctccctcagtggcacattcaacaccagatatgtcttcccccagttagtactaagtgggactcaacttgcacatgaacaacactatgaggtatcaaatgatggtcgtctgaagagcagaagtggtgtctcattgcctgttttagtgatggcgctatatgggagagtatttgagaaggacccacatcgtttaggacagggtgatcatccttga

SEQ ID NO: 123 - DNA, 1347 bp, Puma yagouaroundi
  source 1..1347: mol_type = genomic DNA
atgaacaagaacatagatgttttggagaacgcagttaaactggcagccaggcagggcacacatatcattgttaccccagaagatggaatttatggctgggttttcacaagagagaccatttacccctatctggaagagatcccagacccagaggtgaactggattccatgtacagatccccagaggtttggctacaccccagtgcaacaaaggctcagctgcctggccaaggacaactccatctacgttgtggcaaatattggggacaagaagccatgcaatgccagtgaccctcagtgtccccctgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagcgtcctctacccggtggcgtggtacaacacgctgccccttctgtcggctgttcccttccactctgcgtgggccagaaccatgagagtcaacttgcttgctgcgaatacccacaacaccagcatgcacatgacagggagtggaatctacgcaccggaagcggtcaaagtgtatcactatgacatggaaacagagagtggccagctgatgctgtccgaactgaagtcccggccgcgaagtgaacccacctatcctgcagctgttgactggagtgcttatgccagaagtgtcaagccattcttgtctgaacagtcaaattttccggggatgatttattttgatgagctcaccttcattgagcttaagagaaacacaggaaattacacagtttgccagaaagacctgtgctgtcacttaacttacaagatgtcggagaaacgaacagatgaggtttacgcactaggtgcttttgacggactgcacacggtagaaggtcaatattacctacagatatgcacattactgaagtgtcaaatcagtgacctgagaacttgtggggagcccatggggtcagctttcaccaagttggaagatttctccctcagcggcacatttgcaacacgtcatgttttcccacagatcattctcagtggaagtcaacttgcgcctgaaaggcattatgaggtcttgagagacggacgtctgaggagccgaagcagaacccctctgcctgtcttagttttggccctgtacggaagagtgtttgacaaagaccctccgcgcttggggcaaggaccccggagattagaataa

SEQ ID NO: 124 - DNA, 1545 bp, Microcebus murinus
  source 1..1545: mol_type = genomic DNA
atgatcacttctccgttcctggcttatgtggcaatttgggttttctgtgtcttgagaaccagctctctggacacttttattgcagctgtttacgagcatgcggtgctattgcctaatgccagcctcacaccagtatctcgtgaggaggcgctgaaatggatggatcagaatctggatattttggaaggagccatcacgtcagcagcagagcagggtgcacatattatcgtgactccagaagatggtatttatggctggcacttcagcagggaatctgtctacccgtacttggaggatatcccagaccctcaagtaaactggatcccctgtgataatcctaacagatttggccacaccccagtacaagaaagactcagctgcctggccaaggacaactctatctatgttgtggcaaatattggggacaagaagccatgcaatgccagtgaccctcagtgtccccctgatggccgttaccaatacaacactgatgtggtatttgattctcagggtaggctggtggcacgctaccataagcaaaatcttttcatggttgaaaatcaattcaatgcacccaaggagcctgagattgtgactttcaacaccacctttggaaggtttggcatttttacatgcttcgatatactcttccatgatcctgctgttaccctggtgaaagacttctacgtggataccatactcttcccaacggcttggatgaatgttttgccacatttgtcagccattgaattccactcagcgtgggctatgggcatgggggtcaatttccttgcatctaatacacattacccctcaatgaggatgacaggaagtggcatctatgcccccaattctgcaagagcatttcattatgatatgaagacaaaggagggaaaactcctcctctcacaactggattcccacccacgccaccctgtagtggtgaattggacatcttatgccagcagtgtggaagcactctcaactggaaaccaggaatttaagggcattatcttttttgatgaatacaccttcgtggagctcaaaggagttatggggaattacacagtttgtcagaaagatctctgttgtcatctgaactacaagatgtcagagaaaagatcagatgaagtttatgccctaggggcatttgatggactgcacacggtggaaggacgctattatctacagatttgtactttggtgaagtgtaaaacgactgacttaaacacttgtggtgactcagttgaaacggcttctacccggtttgaaatgttctccctcagtggcactttcggaactcagtatgtcttccctgaggtattgttgagtgagatacagcttgcacctggagaatttcaggtgtcaagtgatggacgcttgtttagcctgaagccaatatctggacctgtcttaacagtgactctgtttgggaggttgtatgagaaggaccgagcatcaaacgcttcatccaacctcatagagcaagcactaagaataatgctagcagttataatatctattgtatactcattacgttggtag

SEQ ID NO: 125 - DNA, 1257 bp, Balaenoptera musculus
  source 1..1257: mol_type = genomic DNA
atggccttcctactgaaggcgcccagcagggaatctatctacccatacttggaggatatcccagaccctcaagtgaactggatcccttgtaataatcccaacagatttggccgcaccccagtgcaagaaaggctcagctgtctggccaaggacaactccatctacatcgtggcaaatattggggacaagaagccatgcaataccagtgaccctcactgtccccctgatggtcgttaccaatacaacactgatgtggtgtttgattctgaggggaaactggtggcccgctaccataagcaaaatcttttcttgggtgaagatgagttcaatgctcccaaggaacctgaggtcgtgactttcaacaccacctttggaaagtttggcattttcacgtgctttgatattctcttccatgatcctgctgtgactctggtgaaagatttccacgtggacaccatactcttcccgacagcttggatgaatgttttgccacatttgtcagctattgaattccactcagcttgggctatgggtatgggagtcaacttccttgcatccaatctacattacccctcaaagaaaatgacaggaagtggcatctacgcacctgattctccaagagcatttcattatgatatgaagacagaggagggaaaactcctcctctcacaactggcctcccacccacaccccacagcagtagtgaattggacttcctatgccagtggtatagaagccccctcaatgggaaaccaggaatttaagggcaccatcttttttgacgagttcaccttcttggagctcaaaggagtcgcaggaaattacacagtttgtcagaaagatctctgctgtcatctaagttacaggatgtctgagaagagatcagatgaagtttatgtactaggggcatttgatggactacatactgtggaagggagctattatttacagatttgtactctgttgaagtgtaaaacaacggacttacacacttgtggtgattcagttgaaactgcttccaccaggtttgaaatgttctccctcagtggcacttttgaaacccagtatgtcttccctgaggtgctgctgagtgaaattcagcttgcacctggagaatttcaggtgtcaagtgacggacgcttgtgcagccagaagccactatctggacctatcttgacagtaactctgtttggaaggttatatgagaaggatagttcaccaaatgctttctcagaccccatggcacagatcatttaa

SEQ ID NO: 126 - DNA, 1617 bp, Rhinopithecus roxellana
  source 1..1617: mol_type = genomic DNA
atgattggatcatattttccaaaatgtgtggcagttgttgccctccttgccttgagtgttggtgcattggacacttttattgctgcagtatatgagcatacagtgatattaccaaacagaacagaaacccctgtttcaaaagaagaaggtttgctcccgatgaacaagaacatagatgttttggagaaagcagttaagctggcagcgaagcgggtattatccttttatacttgtaaaggagacttgcagtttgataaaagagtatttggaattcatgagaaacttttttgccccacttgttttgagcagggtgcacatatcattgtgaccccagaagatggaatctatggttgggtcttcaccagggagagcatttacccttatttggaggatataccagaccctggagtgaactggattccgtgtagagacccctggagatttggcaacacaccagggcaacaaagactcagctgcctggccaaggacaactctatctatgttgtggctaatactggggacaagaagccatgcaatgccagtgaccctcagtgtccccctgatggccgttaccaatacactgatgtggtgtttgattctcagggaaaactggtggcacgctaccgtaagtacaatctttttgcacctgaaattcaattcgatttctccaaggattcagaacttgtgacttttgacactccctttgggaagtttggcatttttacttgctttgacattttttctcatgacccagctgttgtggtggtggatgagtttcaagttggcagcattctctaccccacagcatggtacaacacgctgcccctcctctcggctgttcccttccattcagcatgggccaaggccatgggagtcaatctacttgctgcaaatacccacaacaccagcatgcatatgacagggagtggaatctacaccccagaagcagtcaagatgtaccactatgacatagaaacagagagtggtcagctgttgctatcagaactgaagtctcagccttgccgtgagcccacctaccctgcagctgttgactggcatgcgtatgccagcagtgtcaaaccgttttcctctgagcagtcagattttccggggatgatttattttgatgagtttaccttcactgagcttaagagaaatataggaaattacacagtttgccagaaaaatctgtgttgtcacttaacttacaagatgtctaagaagcaagcagatgaggtctatgctctaggtgcttttgatggactgcacacagtagaaggccaatattacttacagctagaaatgcttaaatatatgtgcacattactgaagtgtcaaaccactgacctggaaacgtgtggagaagctgtggggtcagcttttaccaagtttgaggacttctctctcagtggcacattcggaacgcattatgttttcccacagctcattctaagtgggagtcagcttgcccctgaaagacattatgagatttcaagagatggacacttgaggagccgaagtagagcccctttgcctgtcttagttatggccctgtatggaagagtgtttgagaaggaccctccacgcttagggcagggacccgggaagttccagtga

SEQ ID NO: 127 - DNA, 1284 bp, Felis catus
  source 1..1284: mol_type = genomic DNA
atgactgtgactccagaagatgcgctttatggatggaaattttccagagaaaccatttctctttatctgaaggatatcccggcccctcaggtgaattggattacatgtcaaaacacccagaggtttggctacaccccagtgcaacaaaggctcagctgcctggccaaggacaactccatctacgttgtggcaaatattggggacaagaagccatgcaatgccagtgaccctcagtgtccccccgatggccgttaccagtacaacactgatgtggtgtttgattctgagggtaggctggtggcacgctaccataagtacaatctttttgcgcctgaaattcaatttgatttccccaaggattcagaatctgtgacttttgagactccttttgggaagtttggcattttcacttgctttgacattttttctcacaccccagctgtggtggtggtggatgagtttcaagtggacagtgttctctacccggtggcgtggtacaacacgctgccccttctgtcggctgttcccttccactctgcatgggccagagccatgagagtcaacttgcttgctgcgaatacccacaacaccagcatgcacatgacagggagtggaatctatgcaccggaagcagtcaaagtgtatcactatgacatggaaacagagagtggccagctgatgctgtccgaactgaagtcccggccgcgaagtgaacccacctatcctgcagctgttgactggagtgcttacgccagaagtgtcaagccattcttgtctgaacagtcaaattttccggggatgatttattttgatgagctcaccttcattgagcttaagagaaacacaggaaattacaccgtttgccagaaagacctgtgctgtcacttaacttacaagatgtcggagaaacgaacagatgaggtttacgcactaggtgcttttgatggactgcacacagtagaaggtcaatattacctacagatatgcacattactgaagtgtcaaatcagtgacctgagaacttgtggggagcccgtggggtcagctttcaccaagttggaagaattctccctcagcggcacatttgcaacacgttatgttttcccacagatcattctcagtggaagtcagcttgctcctgaaaggcattatgaggtcttgagagacggacgtctgaggagccgaagcagaacccctctgcctgtcttagttttggccctgtacggaagagtgtttgacaaagaccctccgcgcttggggcaaggaccccggagatttgaatga

SEQ ID NO: 128 - protein, 500 aa, Mus musculus
MASLHFPQWAVSFVFFAQAVGSMDTFIAAVYEHAVILPNKTESPVSTEEALLLINKNIDILESAIKLAARQGAHIIVTPEDGIYGWIFTRETIYPYLEDIPDPEVNWIPCRDPRRFGYTPVQERLSCLAKENSIYIMANIGDKKPCNATDPHCPPDGRYQYNTNVVFDSKGRLTARYHKYNLFEPEIQFDFPKDSELVTFDTPFGKFGIFTCFDIFSYDPAVVVVKDTQVDSVLLPTAWYNTLPLLSAVPFHSVWARAMGVNVLAANTHNTSMHMTGSGIYSPEAVRVYHYDMETESGQLLLSELRSRPRQHATPAEVNWSAYARTVKPFSSGQADFPGKIYFDEFSFTKLTGSAGNYTVCQKDLCCHLTYKMSESRMDEVYVLGAFDGLHTGEGQYYLQICTLLKCQTTNSRTCGEPVGSAFTKFEEFSLSGTFRTKYVFPQIVLSGSQLALERYYEVSRDGRLRSRGGAPLPILVMALYGRVFERDPPRLGQGPGKLQ

SEQ ID NO: 129 - protein, 500 aa, Mus caroli
MASLHFPQWAVSFVLFAQAVGSMDTFIAAVYEHAVILPNKTESLVSTEEALLLINKNIDILESAIKLAARQGAHIIVTPEDGIYGWIFTRETIYPYLEDIPDPEVNWIPCRDPRRFGYTPVQERLSCLAKENSIYIVANIGDKKPCNATDPHCPPDGRYQYNTNVVFDSKGRLTARYHKYNLFEPEIQFDFPKDSELVTFDTPFGKFGVFTCFDIFSYDPAVVVVKDAQVDSVLLPTAWYNTLPLLSAVPFHSAWARAMGVNVLAANTHNTGMHMTGSGIYSPEAVRVYHYDMETESGQLLLSELRSRPRQHATPAEVNWSAYARTVKPFSSGQADFPGKIYFDEFSFTKLTGSAGNYTVCQKDLCCHLTYKMSESRMDEVYVLGAFDGLHTVEGQYYLQICTLLKCQTTNSRTCGEPVGSAFTKFEEFSLSGTFRTKYVFPQIVLSGSQLALERYYEVSRDGRLRSRGGAPLPILVMALYGRVFERDPPRLGQGPGKLQ

SEQ ID NO: 130 - protein, 500 aa, Rattus rattus
MVSLYFPQCVVSFVLFAQAMGAMDTFIAAVYEHAVILPNKTESPVSNEEALLLINKNIDILENAIKLAARQGAHIIVTPEDGIYGWIFTRETIYPYLEDIPDPEVNWIPCRDPKRFGLTPVQERLSCLAKENSIYIVVNIGDKKPCNATDPQCPRDGRYQYNTNVVFDSEGRLTARYHKFNLFEPEIQFDFPKDSELVTFDTPFGKFGVFTCFDIFSYDPAVMVVKDSRVDSVLLPTAWYNTLPLLSAVPFHSAWARAMGVNVLAANTHNTRMHMTGSGIYSPDAVRVYHYDMETESGQLLLSELRSRPHQHGSPAEVDWSAYARTVKPFSSEQADFQGTIYFDEFSFTKLTGSAGNYTVCQKDLCCHLTYKMSESRMDEAYVLGAFDGLHTVEGQYYLQICTLLKCQTTNLRTCGEPVGSAFTKFEEFSLSGTFGTKYVFPQIVLSGSQLALERYYEVSRDGRLRSRGGAPLPVLVMALYGRVFERDPSRLGQGPGKVQ

SEQ ID NO: 131 - protein, 510 aa, Peromyscus californicus
MVPLYFPKCVVSFALFAQAVGALDTFIAAVYEHAVILPNKTETPVSAEEALLLMSKNIDVLEKAIKLAAEQGAHIIVTPEDGIYGWVFTRETIYPYLEDIPDPEVNWIPCRDPKRFGYTPVQERLSCLAKDNSIYVVANIGDKKPCNATDPQCPLDGRYQYNTNVVFDSEGRLTARYHKYNLFEPEIQFDFPKDSELVTFDTPFGRFGIFTCFDIFSYDPAVVTVKDFQVDSVLYPAAWYNTLPLLSAVPFHSAWARAMGVNLLAANTHNTTMHMTGSGIYSPEAVGVYHYDMETDSGQLLLSELKSRPRQSTPAAEVDWSAYARSIKPFSSEQADFPGMLYFDEFSFTKLSGSTGNVTVCQKDLCCYLTYKMSEKRMDEAYVLGAFDGLHTVEGQYYLQICTLLKCQTTNLRTCGEPVGSAFTKFEEFSLSGTFGTNYVFPQLVLSGSQLALERYYEVSRDGRLRSRGGVPLPVLVMALYGRMFEKDPPRLGQGPRKLQRNPFTGDCQD

SEQ ID NO: 132 - protein, 501 aa, Molossus molossus
MIASYFPKYGAVLALFVLSVGALDTFTAAVYEHAVILPNRTETPVSKEEALLLMNKNIDVLEKAVKLAARQGAHIIVTPEDGVYGWVFTRETIYPYLEDIPHPEVNWIPCKDPQRFGYAPVQERLSCLAKDNSIYVVANIGDKKPCNASDPQCPPDGRYQYNTDVVFDSEGRLVARYHKYNLFAPEIQFDFPKDSELVTFDTPFGKFGIFTCFDIFSHDPAVVVVTEFQVDSVLYPTAWYNTLPLLSAVPFHSAWARAMGVNLLAANTHSTRMHMTGSGIYAPEAVKVYHYDMETESGQLMLSELRSRPRREHAYPAPVDWSAHAKGVKPFSSEQSDFPGMIYFDEFTFTELKRKAGNYTVCQKDLCCHLTYRMSEKRTDEVYALGAFDGLHTVEGQYYLQICTLLKCQTTDLGTCGEPVGSAFTKFEEFALSGTFGTSYVFPQLILSGSQLAPERHYEVSRDGRLRSRSGAPLPVLVMALYGRVFEKDPPRLGQGPGKCQ

SEQ ID NO: 133 - protein, 501 aa, Homo sapiens
MIISHFPKCVAVFALLALSVGALDTFIAAVYEHAVILPNRTETPVSKEEALLLMNKNIDVLEKAVKLAAKQGAHIIVTPEDGIYGWIFTRESIYPYLEDIPDPGVNWIPCRDPWRFGNTPVQQRLSCLAKDNSIYVVANIGDKKPCNASDSQCPPDGRYQYNTDVVFDSQGKLLARYHKYNLFAPEIQFDFPKDSELVTFDTPFGKFGIFTCFDIFSHDPAAVVVDEVSIDSILYPTAWYNTLPLLSAVPFHSAWAKAMGVNLLAANTHNTSMHMTGSGIYAPEAVKVYHYDMETESGQLLLSELKSRPRREPTYPAAVDWHAYASSVKPFSSEQSDFLGMIYFDEFTFTKLKRNTGNYTACQKDLCCHLTYKMSEKRTDEIYALGAFDGLHTVEGQYYLQICALLKCQTTDLETCGEPVGSAFTKFEDFSLSGTFGTRYVFPQIILSGSQLAPERHYEISRDGRLRSRSGAPLPVLVMALYGRVFEKDPPRLGQGSGKFQ

SEQ ID NO: 134 - protein, 501 aa, Felis catus
MITSYFPKCVALLAICVLCVGALDTFIAAVYEHAVLLPNRTETPVSKEEALLLMNKNIDVLENAVQLAARQGAHIIVTPEDGIYGWVFTRETIYPYLEDIPDPEVNWIPCTDPQRFGYTPVQQRLSCLAKDNSIYVVANIGDKKPCNASDPQCPPDGRYQYNTDVVFDSEGRLVARYHKYNLFAPEIQFDFPKDSESVTFETPFGKFGIFTCFDIFSHTPAVVVVDEFQVDSVLYPVAWYNTLPLLSAVPFHSAWARAMRVNLLAANTHNTSMHMTGSGIYAPEAVKVYHYDMETESGQLMLSELKSRPRSEPTYPAAVDWSAYARSVKPFLSEQSNFPGMIYFDELTFIELKRNTGNYTVCQKDLCCHLTYKMSEKRTDEVYALGAFDGLHTVEGQYYLQICTLLKCQISDLRTCGEPVGSAFTKLEEFSLSGTFATRYVFPQIILSGSQLAPERHYEVLRDGRLRSRSRTPLPVLVLALYGRVFDKDPPRLGQGPRRFE

SEQ ID NO: 135 - protein, 503 aa, Tachyglossus aculeatus
MIFTAFPVYAVFFALWAPGARTEDSFIAAVYEHAVILPNGTQTPVTQDDALMLMDKNLDVLEKAIKQAASEGAHIIVTPEDGIYGWVFTRDTLYPYLEDIPDPQVNWIPCTDPNRFGRAPVQKRLSCLARDNSIYVVANMGDKKPCNTTDSQCPRDGRYQYNTNVVFGADGKLVARYHKYNLFEPEVQFDFPKDSQDVTFETPFGKFGIFTCFDIFSHDPAVTVVEKYQVDSVLYPTAWDNTLPLLSAAPFHSAWARSMRVNLLAANTHNTSMHMTGSGIYAPDGIRAFHYDMETESGQLLLSELKSRPRLWPSYPVAVDWSAYAKHVKPPSREYKKFGGKIYFDEFTFTDLIQAGENYTVCENDLCCHLSYKMTEKRADEVFVLGVFNGLHIVEGQYYLQVCTLLKCQTTDLRTCGEPIGSAFTKFEEFSLSGTFGTNYVFPQVILSGSQLATEQQYEVTSDGRLRNRGSVPLPILVMALYGRVYEKDPPRLGQGVRPLRPL

SEQ ID NO: 136 - protein, 483 aa, Myotis lucifugus
MITSSFQASVAVLALVTLHVSALDTFIAAVYEHAVLRPKGTETPVSRDDALLLMNKNIDILEKAIKQAAEQGAQIIVTPEDALYGWKFTRETIFPYLEDIPDPQVDWIPCQDPQRFGHTPIQTRLSCLAKNNSIYVLANIGDKKRCNSQDSTCPPNGYYQYNTNVVYDAKGQLVARYHKYHLYSEPQFDVPEKPELVTFNTTFGRFGIFTCFDILFHDPAVSLVKDFHVDTILYPTAWMNVLPLLSAIEFHSAWAMGMRVNLLAANIHHVQQNMTGSGIYAPHSPRVYHYDMETDSGRLLLSEVDSRPRTSPAYPTAVDWSAYATTIKPFPVQKNTFSGFISRDAFNFTALFENAGNLTVCHKDLCCHLSYRMLRKEENEVYVLGAFTGLHGRRRREYWQVCTMLKCKTTDLTTCGQPVETALTRFDIFSLSGTFRTEYVFPEVLLTKVHLSPGKFEVGGLEEYFYFICSLKPSKIGYGNCIT

SEQ ID NO: 137 - protein, 514 aa, Equus asinus
MISSQLLAYVAVLVFCTFQARSLETFIAAVYEHAVILPNATLTPVSREEALLLMNRNLDLLEGAIASAAKQGAHIIVTPEDGVYGWNFSRDTLYPYLEDIPDPEVNWIPCNNPHRFGHTPVQERLSCLAKDNSIYVVANFGDKKPCNASDPECPPDGRYQYNTDVVFDAQGKLVARYHKQNLFMGEDQFNAPKEPEVVTFNTTFGKFGIFTCFDILFYDPAVTLVTDFQVDTILFPTAWMNVLPHLSAIEFHSAWAMGMGVNFLASNIHYPSMKMTGSGIYAPDSPRAFHYDMKTEEGKLLLSQLDSHPHHPAVVNWTSFASGVKTLSTGNQEFNSTVFFDEFTFLELTEVTGNYTVCQKALCCHLSYKMSEKRSDEVYALGAFDGLHTVEGSYYLQVCTLLKCRTTDLHTCGDSVETASTRFDTFSLSGTFGTQHVFPEVLLSEIQLAPGEFQVSSDGRLFSLQPPSGPVLTVTLYGRLYEKDQVSDASSDLTAQALRVMLVVLIPVGFSISW

SEQ ID NO: 138 - protein, 514 aa, Egretta garzetta
MLHKKHSRSCEAAAMLPSQSLLHAAVFALTVLQALASDTFIAAVYEHAVILPDATDEPVSPDDALALMNKNMDVLEGAVKEAAQQGARIIVTPEDGIYGWRFTRETIYPYLEDIPDPAVNWIPCTDPTRFAPAPVQERLSCMARSNSIYVVANIGDKKPCNSSDAGCPRDGRYQYNTNVVFDSEGKLVARYHKYNLFMSETQFNYPKEPEAVTFETPFGKFGIFTCFDILFREPAVVLVSELQVDTVLFPTAWMNVLPFLTAVEFHSAWAMGMGVNFLAANTHYTNISMTGSGIYAPNGARTYYYNMKTEDGHLLIAELDSHPRLSPASPPAVSWSSYASSVESFSPNDHDFGGLIFHDWFTFTELTKPEGNLTVCQKDLCCHLSYKMAGKQKDEVYVLGAFDGLHVVEGQYYLQICTLLKCKNTDSNTCGQPVESAQTRFERFSLSGTFGTSYVFPEVLYSGVQLAPGEFEVLNDGRLISKKRTTKPVVTVTLFGRWYEKDHLKQDPQSPAFP

SEQ ID NO: 139 - protein, 520 aa, Bos javanicus; identical to SEQ ID NO: 85

SEQ ID NO: 140 - protein, 521 aa, Sus scrofa
MIASSFQTTLAVFALIILHVRALDTFKAAVYEHAVILPNDTKTPVSWDDALLLMNKNMDILERAIKQAAEQGSQIIVTPEDALYGWKFTRETIFPYLEDVPDPQKNWIPCQDPRRFGHTPVQVRLSCLAKNNSIYVLANIGDKKPCNSHDSKCPPNGYYQYNTNVVYDSEGKLVARYHKYHLYHELQFDVPEKPEVVTFNTPFGKFGIFTCFDILFRDPAVTLVQDFHVDTILFPAAWMNVLPLLTAIEFHSAWAMGMRVNLLAANVHHVRLNMTGSGIYAPHSPKVYHYDMETDSGKILLAEVDAHPRTSPTYPTAVNWGAYTLSIQPFPILQNSFRGFISRDEFHFTELSESAGNLTVCHKELCCHLSYRMLAKEENEVYVLGAFTGLHGRRRXEYWQVCTMLKCKSTNLTTCGQPVETALTRFELFSLSGTFGTEYVFPEVLLTKINLAPGKFEVLKDGRLVNKNGTSEPVLGVSLFGRWYIRDVPLSISGGPAPQQKMYLIISALLMIIALQNIVMV

SEQ ID NO: 141 - protein, 546 aa, Bos indicus
MLHKCSFLYKYYMNVTFTCRTQDTLAMITSSLQTSVAVFALXILHVSTLDTFKAAVYEHAVILPNDTETPVSPDEALFLMNKNIDILEKAIKQAAEQGAQIIVTPEDALYGWKFTRETIFPYLENIPDPQVNWIPCQEPHRFGHTPVQARLRSLAKNNSIYVVANMGDKKPCNSHNPXCPSNGHYQYXTNVVYDAKGKLVARYHKYHLYREIQFDVPEKPELVTFNTTFGKFGIFTCFDILFHDPAVTLVKDSHVDTILFPTAWMNVLPFMTAIEFHSAWAMGMRVNLLAANTHNVSLKMTGSGIYAPHSPKVYHHDMETESGKILFAEVDSHPRNSSTYPPAMNWSAYATSITPFPGPKNSFRGFISRDEFNFTKLSESAGNLTVCQKELCCHLSYRMLAKEGDEVYVLGAFAGLHGRRRREYWQVCTMLKCRSTDVTTCGQPVETALTRFEMFSLSGTFGTEYVFPEVLLTKIKLAPEKFEVLEDGRLVNKNGLSEPVLTVSLFGRWYXKDAHSISGGTSNSATAYLMIPTLLMIITLRDAVMX

SEQ ID NO: 142 - protein, 520 aa, Patella sp.; identical to SEQ ID NO: 100

SEQ ID NO: 143 - protein, 561 aa, Biomphalaria sp.
MGKEVRLLLLLLGTLSSVLANRSKFKGAVYGHAVLLPKDSLKVISRAEALDNMNKNLDVYREQVTKAKSQGAEIIVFPEDGLYGFQFTRQSIYPYLEYIPDPRTELWSPCDEPGRYNDTEVQRSLSCLAKTNSLYLVANMGDKQPCDAATDRTCPPDGRYQYNTNVVYGPDGRLLARYHKYNLFYELQFNAPELELSYFDTPFGRFGLMICFDVLFHDPGLPLILSLNISNIIFTSAWKDALPLLSALGYHSSFARGLGINMLGANIHIPDYRFHGSGIYGPDGTIISFYGSTNASKPKLLVADVNVLDDTQPNKIVSSIVQQNLKQARMNEADDKRVTKDTTKNSKKTFESLLFYDMFTFAALDKPSGHIKVNQKHVHCELRYSIPEELFGRELYAFGAFDGLHTYEGQYYFQICALVKCADARNRSTCGSVTYDAITPFVRLSISGEMQTPFVYPQIILSNGIGELSLAEPDSWTYTGKSLKTDKEFYKPVLSAALFGRDYFRDTDGQNKGDNLYKSQEDATDKEDKSEIYLGLVSGQNIFIFLLGFVCLLLLVNMAIF

SEQ ID NO: 144 - protein, 501 aa, Mya arenaria
MTVGSPGMLLTLLLVLGGRILAGLETPLADSYRAAVYEHNVSFPRHRDVVPRLEALQLMEENLGKFDEQARQAAQQKAKIIVFPEYGVYGLGWTRQKLAPFLEYIPDPAGNPWNPCDNPNGFVSAVQTLLSCIAKNNSIYVVANIGSQQPCISTDKSCPSDHVYQFNTNVVYDPRGIFIARYFKYNLDSVEMQYFDRPPVVNHTIFDTPFGRFATVCSNDILYKEPTLTLVKTMNITNLAMPAAWKEELPLMSAIEFHSAFAEGLLINVLAANLHVVDLGYYGSGLYWPTGTSINASYFYDDDPYSEGSLVVETLTPSIIPSDVWDPPQFTENKRFAPRRLVNNAEQSSLTHKIKINGDIYTAKVFKFGEAYSANVCQGSLCCDADFEGGASGDMFAFGAFEGMHSGNNSYYLQSCALIKCAGRTVESCGQPTKHTSTGIDKMSMSGNFSYPYQFPEVFTVGDHPEIVTQIWYYQGSVIIDVGLYGDLLTVAIQAIDYSRQ

SEQ ID NO: 145 - protein, 537 aa, Dissostichus mawsoni
MKHLYSRTQMLFMSLREGTMFLSVIVFSSLSVSTALGRTEPVVDSSYIAAVYEHNIILNPDPRVPLSRPAALQHMQRNLDIYEQQAARAAQQGVQILVFPEDGIQGFNFSRSSIFNYLETIPDPQQESWNPCTEPGKYNNTEVLQRLSCMARRHNLYLVANMADLQPCPLKTDPSSSCPSDGRWQFNTNVVFRSDGLLVGRYHKNNLYFEAAFDTPPQPEIITFDTPFAGRFGLIICFDILFYEPKVHLVTKGVRQLIFPTAWMNQLPLLDAIQFQHAFSLGANVTLLAANIRNDQMIMTGSGIYTPFSVKSHHAQKGDPDEGRLLVARVPVLDPLWAGQSVTDGSTAKDSGFCHKESCLDFPAPETSSSVPPSSSASSTSTFISSMMYDPFTFVLLEATEGKVNVCNGTFCCHLQYRLIPQGDSKELYALGAFGGTHTVNGRYAVQVCALVRCAGLDTSSCGQEVEEAESKMDFQLEGRFASRYVYPSVLASKFVLEQPKHFETAPDGTVTMKHSNMTGGLVTACLYGRMYHLENE

SEQ ID NO: 146 - protein, 620 aa, Priapulus caudatus
MAPIRLQCLRVGSPHRRALVVAAVLLATLQYPSATATARRTSSDDSSCTYTAAVLQYYLQTPGPGSDPSEVMRSNLLAYEEYLMQAAAQGASIIVFPETGITGFLQKDREIVLKWLQEIPEPNTEAALDGQRSSDSPDSCDSGCVATSGDDFVLESLKCMAACHKITIVANLGEVQYCQPAASSTVPTQVQSSAAAEDAVPGCPPDGRFQFNTNVVVDDNGALVVRYRKQHLYNEAAFDDPPVPEAGVFDTPFGRFTTFVCFDINFYNTSADVVLHDGVDSVAMSAAWPIALPTSSPQQTHSGWALGTGVNLLTANVRGVTDGLTFGGSGIYSGVSGNLTYTREDAPMQPELVIADVPCYRKRSWESYAHRPYSVEIYDAPVAAIARPAGGGGSGNKRRMKNKKNSRKRNEHRRRPGNEPNSENSETGDNSPRTGGEREQHTSAPPPKYFKNYVIGVLFNFTALEADIGRASVCYGDLCCHLDYAMSARGRRSDELYALAAFNDYYTAVTDQFWWQVCMVVKCRTSSYKTCGESVTRAKTKFKALTLSGDFWVEFISPSVRVDGDYLLAPLDQWSFDNDGEVMFSTKGIADPLITAEIQAFEPEKHDQSKRGVDVTCTCT

SEQ ID NO: 147 - protein, 526 aa, Ruditapes philippinarum
MDYYLWNMEAKKNVVCCFLLSIGISMITSAKVKVAVYEHVVISPDDPEALFTRQEAFQWMSKNLNVFVEQARKASEQGAKIIVFPEYGITGFDHSRNSIVPFLEDIPDSSTDWNPCMEPDRFADTLVLRNLSCTAQMYKIYIVANMGDMKTCDKMTDETCPHDGRYQFNTNVIFDNNGQLVARYHKLHMYDETPLFDPAPNAEHVYFDTPFGRFGTIVCFDIMQNEPTQVLLNKYSITNLLVTSAWNVFLPFIIPQQMYSGLAKKSGINVIASNIRNSLYQMAGSGIFGNDIQSLSDVNSNGAEGILLVSEIETEEAKIYAPNTIMSINSSGKVKQFINKTYEGTYRFGLNMSYAIMEGQSGIVTSCKDDVCCVVTYQFKSRDENETIILSVVDELLYKPGIIHMQYCAVFKCSPHTIYSCDYDDIFVINSIFADIKIQGYFDNGVTFPLVTTIDNNELTMDMDKYTFDDKNAILHSNQFENPLLAAVVYNTLHLDVIKTGASSNVAQGTTLSLSTFLLIYIYKAS

SEQ ID NO: 148 - protein, 511 aa, Paramuricea clavata
MAKQTLFFMLFVLSISSRILAKEYKVATFEHHPFFILAPEPLSREKALQMMRKNTAVYSEQARAARKQGAQIIVFPEDGIYGFFHSRETIKPYLEPIPTVSNTTSIIPCTDPKPNTEILQQLSCIARNNSIAVVANMGDIQYCSQRDPNCPKDGRYQYNTDVIFDKDGRLLAKYHKQHLYFEKAFNTPTKCEFVTFVTSFQVRFGVFTCFDMLFKDPAIDLVQTYGVQNIVVPTAWYKGFPALVSIEYHQAWSRVNCVNLVTANVYYPNRGLNFTGTGIYDCGEAKTYTYGEETIEKRLLFATLSGNREAQNDKTDMLYRWRRDIEMVKEFGMDRKIPQDIDNENRMLDDPYDSIKTFKAKIMGNVFTLTKLTSPASELSVCVKGLCCNLKYGLPEGKQFTETFAMGTFIGNNPDGFYWEVCLLLKCASSEESSCGTTVMDSGTVFQSFTMSGNFSSTALVFPMALGSGVSLFSTDKVKFANKSISGKALQKPLLTASLLGRVYSKDKPHN

SEQ ID NO: 149 - protein, 559 aa, Nilaparvata lugens
MGDIQYSIFKSEIHNHNNAVERKKMISTVRSFQRLFLLFLVACTLFATSIIVNGYLNSGRDKYVAAVVEYAPLEWDGFEKPKDLFLRNANRFVTIIERAAKENADIIVFPECGLTTLNIPVKREGITEYTTFIPNPVEKRTPCTDNDTGLAEHLRVLSCAAKNNKMYVAVNMDEKITCESDMQGCPKDHLIFFNTNIVFDRNGTIIAKYRKFNLFKEPEFNSTIFPIYAVFKTDFGATFGMFICFDILFAQPAEVIVSKYKVTDILYSTAWFSELPFLTAVQTQAGYSYSMNVNLLASGYNRPDLYSGGSGIYAGEDGPVVTTMPAFRTTRLLVGELTIYGTGRKRPVCEYADSCPLSKGKVIDIHFNDTHDKEGMLFLKDDLSKYSTEFIEETTSYEHAMIQHLGDDKETNLEYSRNGFNCSITVTWFRDDTSSNNKGPVYRLVAHSGIRSFAGVRSAYIEVCALVACADRTIESCGHRPSFENVSSRTQVKNVNIHTESLNITTPFVSTLDSNIYPLMGNELSFIESIDKFRGYIQKTYLFLEKPKDDLITFGVYKL

SEQ ID NO: 150 - protein, 512 aa, Ischnura sp.
MGLLHGILFVLLIPLCAAHRKTYRAAVVEYHPLGDRHDNASHILNLNVAEYETLIEEAKRKGADIVVFPEYGLTQLAASSSRNLAIQFAQSVPTPPAPGTPSSAAQVPCLLPNSSMAIRRLSCSARENQIYVVVNLLEIAPCSITDHACPEDGFFIYNTNVAFDRNGGVVSKYHKYNLFKEPSVNTPKHSMYSTFDTDFGVTFGMFICFDILFEDPGYTLVKRMNVNNMIYSTAWISELPFLTATQTQQSWSLGLGVNLLASGFNNPMGGNGGSGIYAGGSGRLNLLMPSTAGTTLLLADVPVVSSNRLRGDSLSEPELSLDLQTGNQATSGHMFVLHDDLSNYKSSEVLKNGLNELNMCQNNLCCNFKADVQLSSNGSQAPKYRFVITDSIIKYARTYPAHVQVCGLIYCKGDDVSKCVYFPDLPETLDNVSFRSLGISGNFSSPRMVLPSTLTTSLEPLKISSGNLMEKDEAITLEYNSDTRGLLTFALYGRIFQQNMIRNEEIMDNDIY

SEQ ID NO: 151 - protein, 533 aa, Argiope bruennichi
MKLPGIQFLCFFVMLSMGSAQMRSRRPYYRAAVHEHKQYDDKTSSGREIINKNLRKYEVAARLAARNYVDLIVFPEKGLFPMKMDNPTWFLDYAENIPRGDEMTNPCYQEKFSSSPILTNLSCIARKYSLFVVATLIDVKDCKVRRHCENRKNKNSCVSDGSDCPDNGHFNFNTLVVFDRMGTLIVRYYKRHPFIPLEKGISTPQYPESRYFDTEFGSFATDIGFDFLINDSFIDIAKRPETTGVTYGNWWFDHTPFFYFSVPNQQAWCLTNKVTVLSSDVHSPNLASLGSGIYIPGKGAVIYSYNPDGKSKLLISNIPTSFFYDFQSTLPPLNKRFFYINDDDSISELNGEEPRNFKDKCGENVLGMNPSSLTDYRCKQSEVHQYTFVKLNKTEDYINVCSNNFCCSLEYQAESMDETFYFAVSGNRLKFYDIYWFGVQSCFLSRCEPVDSKPCRNFLLKSNTKFNSVKITGSFDTNHIYPHVLDSELRLTDRDEWKFDGTSQLSYQNLKGKNLLHADLYGRLYREDGLVNN

SEQ ID NO: 152 - protein, 573 aa, Patella sp.
MMYKIMLLRIVATYTCLSLVEGYRPSYRAAVYEHALFQPTHFRTVSRNQALRDMKKNLDIYAEQTRIASSQNVDIIVFPEDGIYGLNFNRDTIKPYLEDIPDPTEITWNPCNQSEYTNGVEVQITLSCMARNNGIYIVANMGDKKVCDADSICPDDGQRQYNTDVVFDPQGNLVARYHKQHLYFEAQFDTPSTVERVTFDTPFGKFGIITCFDILFKSPAVDLVEDLGVKNVIFPTAWMDSLPLLSAIQFHSAFAKLFKVNLLAANLHFPLFNFHGSGIYSPRRTEAYYYNDGLFSPGKLLIADVKSSPTHYRRHQVVPDTHGFRGGNSLGASFVHGQDTAEVNTQSRRNGGHYNTASRNGIISHQLADQASGVHLNNDLGPLTHRSIRNRIHNHAHLHRVNRHMVENGWATIMADNDNYQYLPLTEPKAKVKICQNEVCCYLDYQKQNTSNDELYALGVFDGLHQIEGLSWYTQVCVLLKCEFTDIASCGQPTKIAAGHFEYLKLWGTFTTEYVFPEVLVSDDGKLRLEHEHWKYLHNRIEFVNRGKERPLLSATLYGRHYDRDNSTVSNSV

SEQ ID NO: 153 - protein, 568 aa, Lonchura striata
MLYHDPAVVMASRFQINTILFLTAWVNTLPLLSAAQFYLVQALGMGVNFLSANTHNSTLDMTGSGIYAPDSPRAYYYNTEMDNGHLLVTELSSHPHLSSDYPVAVNWTLSAIFPCSTCGQPVETVQTKFEMFSLSGIFGISYIFPEVLYSGVQLAPGEFKGAHIIVTPEDGIYGWRFTRESIYPYLENIPDPVVNWIPCTDPSRFGPAPVQERLSCMARNNSIYVVANIGDKKPCDSSDPSCPRDGRYQYNTDVVFDAQGKLVARYHKYNLFLGEDQFNYPKEPEAVTFETPFGKFGIFTCFDILFYEPAVVLVSKMQVDTVLFPTAWMNVLPFLTAIEFHSAWAMGMRVNVLAANTHNTSMEMTGSGIYAPAGARTYSYNKKTEDGHLLIAELDAHPRLSPVSPPAVSWNSYALSVERFSQNDHEFTGIIFEDLFTFTELTKPGENLTVCQKNLCCHLSYKMAEKRDDEVYVLGAFDGLHVIEGQYYLQICTLLKCPSTNLSTCGQPVETAQTKFEMFSLSGTFGTSYVFPEVLYSGVQLAPGEFKVMGHPCDLSDISGQKPTTRAT